(19) United States
(12) Patent Application Publication
(10) Pub. No.: US 2005/0003390 A1
(43) Pub. Date: Jan. 6, 2005

(54) Targets for controlling cellular growth and for diagnostic methods

(76) Inventors: Sergey A. Axenovich, San Jose, CA (US); Robert Stull, Alameda, CA (US); Marina Gelman, San Francisco, CA (US); Kitty Chui, San Francisco, CA (US); Dean Ng, Foster City, CA (US)

Correspondence Address: SHERIDAN ROSS PC, 1560 BROADWAY, SUITE 1200, DENVER, CO, 80202, US

(21) Appl. No.: 10/789,378

(22) Filed: Feb. 26, 2004

Related U.S. Application Data
(63) Continuation-in-part of application No. 10/441,925, filed on May 19, 2003.
(60) Provisional application No. 60/381,619, filed on May 17, 2002.
(60) Provisional application No. 60/450,886, filed on Feb. 26, 2003.

(51) Int. Cl. C12Q 1/68; G01N 33/574

(52) U.S. Cl. 435/6; 435/7.23

(57) ABSTRACT

A method of identifying a compound that induces apoptosis is disclosed. The method includes identifying compounds that inhibit the expression and/or activity of a target. Also disclosed are methods for inducing apoptosis by inhibiting one of the targets. The invention further includes methods for the diagnosis of a tumor that include determining the level of at least one of the targets as a biomarker in a patient sample, the level of the biomarker being indicative of the presence of tumor cells.

### CROSS-REFERENCE TO RELATED APPLICATIONS

This application is a continuation-in-part of U.S. patent application Ser. No. 10/441,925, filed May 19, 2003 and entitled “Cellular Gene Targets for Controlling Cell Growth”, which claims the benefit of priority under 35 U.S.C. § 119(e) from U.S. Provisional Application Ser. No. 60/381,619, filed May 17, 2002, and entitled “Cellular Gene Targets for Controlling Cell Growth”. This application also claims the benefit of priority under 35 U.S.C. § 119(e) from U.S. Provisional Application Ser. No. 60/450,886, filed Feb. 26, 2004, and entitled “Diagnostic Methods for Cancer Detection”. The entire disclosure of each of U.S. Provisional Application Ser. No. 60/318,619, U.S. patent application Ser. No. 10/441,925 and U.S. Provisional Application Ser. No. 60/450,886 is incorporated by reference herein in its entirety.

### FIELD OF THE INVENTION

The present invention relates to methods for inducing apoptosis in cells by inhibiting targets involved in the suppression of apoptosis, and to identifying compounds useful in such methods. The present invention also relates to methods for the diagnosis of cancer in a patient using the targets identified by the present invention as biomarkers.

### BACKGROUND OF THE INVENTION

The p53 tumor suppressor protein is an essential component in the regulation of the cell cycle, senescence, and programmed cell death (apoptosis). This protein regulates transcription of many genes in response to DNA damage and various transforming stimuli. The functional inactivation of p53 can occur through the action of viral oncoproteins, or through over-expression of the hdm2 (human) or mdm2 (murine) oncogene protein. Additional tumor suppressors, such as the P14^{ARF} product of the INK4a gene, regulate the functional activity of p53. In the case of p14^{ARF}, the suppressor interacts with hdm2 and thereby prevents the mentioned oncoprotein from inhibiting p53. An alternative translation product of the INK4a locus, p16INK4a, a cyclin-dependent kinase inhibitor, also contributes to normal growth control through its regulation of the Rb pathway.

When regulation of the cell cycle, senescence, and apoptosis is not functioning properly, uncontrolled cell growth and tumor formation occurs. Because of the complicated regulation of these cell functions, there are many potential points in a variety of regulatory pathways of a cell for intervention. By inhibiting the expression of genes important to cell growth and to suppression of apoptosis or the proteins encoded by them, it is possible to induce control cell growth and apoptosis in a cell, thereby preventing tumor formation. Once such genes or proteins are identified as targets, assays can be conducted for drug discovery to find inhibitors suitable for use as therapeutic agents. In addition, such genes or proteins are useful as markers of tumor formation.

There is an ongoing need to identify new targets and develop new assays for the identification of therapeutic compounds useful in the control of cell growth and tumor formation.

### SUMMARY OF THE INVENTION

This invention provides methods for identifying compounds that induce apoptosis by inhibiting target genes or gene products involved in the control of cell growth. The present invention also includes a method for inducing apoptosis in a cell by inhibiting such a target gene or gene product by, in one embodiment, contacting cells susceptible to uncontrolled growth with an inhibitory compound in an amount sufficient to inhibit said biochemical activity or expression. More particularly, targets of the present invention include any of the genes or gene products set forth in Table 1, which can also be identified as genes and gene products comprising SEQ ID NOs:1-80 (with odd numbered identifiers referring to nucleic acid sequences and even numbered identifiers referring to amino acid sequences).

In one embodiment, the present invention relates to a method of identifying a compound that induces apoptosis in a cell that includes contacting the cell with a putative apoptosis-inducing compound and determining whether the compound inhibits the expression and/or activity of a target selected from the group consisting of any of the targets listed in Table 1 (or comprising any of SEQ ID NOs:1-80). The target can have been validated as being involved in tumor cell growth, such as by a process of inhibiting the target in a cell by a method selected from gene knock-out, anti-sense oligonucleotide expression, use of RNAi molecules and GSE expression, or assaying the cell for the ability of the cell to grow. The cell can be a tumor cell line. The step of determining can be selected from assaying for reduced expression of the target and assaying for reduced activity of the target. The expression of the target can be measured by methods including, but not limited to, polymerase chain reaction or by using an antibody that specifically recognizes the target. The activity of the target can be measured by methods including, but not limited to, measuring the amount of a product generated in a biochemical reaction mediated by the target or by measuring the amount of a substrate consumed in a biochemical reaction mediated by the target. The inhibitor can be identified by methods including, but not limited to, determining the three-dimensional structure of the target or by determining the three-dimensional structure of an inhibitor by using computer software capable of modeling the interaction of the target and putative test compounds.

Another embodiment of the present invention is a method for inducing apoptosis in a cell by inhibiting a target selected from any of the genes or products encoded thereby listed in Table 1 (also represented herein as genes or gene products comprising any of SEQ ID NOs:1-80).

A further embodiment of the present invention is a method for the diagnosis of a tumor that includes determining the level of a biomarker selected from any of the genes or products encoded thereby listed in Table 1 (also represented herein as genes or gene products comprising any of SEQ ID NOs:1-80) in a patient test sample. In this method, the level of the biomarker is indicative of the presence of tumor cells. The presence of the biomarker at an increased level as compared to a normal baseline control is an indication of the presence a tumor, a possible predisposition to such tumor or a susceptibility to an anti-cancer therapeutic treatment. The level of the biomarker can be determined by conventional methods such as expression assays to determine the level of expression of the gene, by biochemical assays to determine the level of the gene product, or by immunoassays. In one embodiment of this method, the level of the biomarker can be determined by identifying the biomarker as a cell surface molecule in tissue or by detecting the biomarker in soluble form in a bodily fluid, such as serum, that can be immobilized. The biomarker level can be determined by contacting a patient test sample with an antibody, or a fragment thereof, that binds specifically to the biomarker and determining whether the anti-biomarker antibody or fragment has bound to the biomarker. The biomarker level can be determined by using a first monoclonal antibody that binds specifically to the biomarker and a second antibody that binds to the first antibody. This method can be used to determine the prognosis for cancer in the patient or to determine the susceptibility of the patient to a therapeutic treatment.

### BRIEF DESCRIPTION OF THE DRAWINGS OF THE INVENTION

FIG. 1 illustrates a schematic of the features of the V98 vector.

FIG. 2 illustrates a schematic drawing of the construction of the V87 vector.

FIG. 3 illustrates a schematic drawing of the construction of the V98 vector

### DETAILED DESCRIPTION OF THE INVENTION

The present invention includes methods for identifying protective compounds that control cell growth and induce apoptosis by using genes that encode products that are necessary for protecting cells from apoptosis as targets in the design of therapeutic agents. The invention further includes compounds for use in the treatment or prevention of tumor growth. Such compounds include chemical compounds and biological compounds. Chemical compounds or biological compounds include any chemical or biological compound that disrupts or inhibits one or more biological functions required for controlling cell growth. Preferred chemical compounds include small molecule inhibitor or substrate compounds, such as products of chemical combinatorial libraries. Preferred biological compounds include peptides, anti-sense molecules and antibodies.

The invention also includes methods for the diagnosis of cancer or for a prognosis of cancer or for determination of susceptibility to cancer treatments, by determining the level of expression of target genes and proteins of the present invention (also referred to as tumor antigens (TAGs)) in patient samples. The targets may originate from different parts of the cell and may be cell surface proteins, intracellular proteins or proteins that are secreted from the cell. There is a distinction between tissue, individual and species-specific cellular markers that may also be present physiologically as differentiation antigens on cells. There may be targets that are intermediate products released, over expressed or under expressed during the growth of a tumor cell type which can change upon further differentiation. The level of the target gene or protein can be determined by conventional methods such as expression assays to determine the level of expression of the gene, by biochemical assays to determine the level of the gene product, or by immunoassays. If appropriate, the marker can be identified as a cell surface molecule in tissue or in a bodily fluid, such as serum. These methods are described in detail below. The present invention provides much needed markers that permit an improved and more specific diagnosis of cancer, including the possible distinction between various tumor types, the prediction of tumor formation and the patient susceptibility to certain known cancer treatments.

The present invention is based, in part, on the present inventors' isolation of certain GSEs from human cells that prevent cell growth, and the discovery that such nucleic acid molecules correspond to fragments of certain genes. In that regard, any cellular phenotype or protein associated with cell growth can be used to select for such nucleic acid molecules or proteins encoded thereby.

More specifically, targets of the present invention have been identified as corresponding to genetic suppressor elements (GSEs) that control cell growth. The GSX™ System technology allows rapid screening for the inhibitors of gene function in the form of genetic suppressor elements. Briefly, a Genetic Suppressor Element (GSE), is a gene fragment, which, when expressed in cells, acts as a genetic inhibitor of the corresponding intact gene in those cells. A GSE can exert its effect through either an antisense, or a dominant negative peptide mechanism. GSEs are selected from libraries of DNA fragments, generated by random breakage of sets of test genes, cloned in a retroviral or other expression vector. The RFL clones are introduced into a population of test cells at approximately one test fragment per cell. Cells with a desired new phenotype, resulting from the expression of a GSE, are isolated on the basis of any selectable parameter. The GSEs are recovered from the selected cells and characterized by DNA sequence analysis and further functional assays.

GSEs having the ability to control cell growth can be functional in the sense orientation (and encode a peptide thereby), and can be functional in the antisense orientation (and encode antisense RNAs thereby). These GSEs are believed to down-regulate the corresponding gene from which they were derived by different mechanisms. Such a corresponding gene is referred to herein as a “target gene” and its product (i.e., protein encoded by the coding region of the gene) is referred to as a “target product” or “target protein”. As used herein, the term “target” alone can refer collectively to a target gene and its corresponding target product, or to useful portions thereof. Sense-oriented GSEs exert their effects as transdominant mutants or RNA decoys. Transdominant mutants are expressed proteins or peptides that competitively inhibit the normal function of a wild-type protein in a dominant fashion. RNA decoys are protein binding sites that titrate out these wild-type proteins. Anti-sense oriented GSEs exert their effects as antisense RNA molecules, i.e., nucleic acid molecules complementary to the mRNA of the target gene. These nucleic acid molecules bind to mRNA and block the translation of the mRNA. In addition, some antisense nucleic acid molecules can act directly at the DNA level to inhibit transcription.

Specific targets of the present invention are shown below in the Examples section in Table 1. The targets include the genes and products of the genes or any useful portion thereof. Methods of the present invention for identifying therapeutic compounds by identifying an inhibitor of a target include identifying an inhibitor of: a target gene from Table 1, as well as target products encoded by any of the foregoing. Diagnostic methods for detecting cancer in a patient include detection of a target gene from Table 1, as well as target products encoded by any of the foregoing, and useful portions thereof. More specifically, the targets of the present invention include genes comprising all or a portion of any of the nucleic acid sequences represented by SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, or 79. These nucleic acid molecules encode the following target proteins, respectively: angio-associated, migratory cell protein (AAMP; SEQ ID NO:2), a disintegrin and metalloproteinase domain 8 (ADAM8; SEQ ID NO:4), a disintegrin-like and metalloprotease (reporlysin type) with thrombospondin type 1 motif, 17 (ADAMTS17; SEQ ID NO:6), adenylate cyclase 3 (ADCY3; SEQ ID NO:8), adrenergic beta receptor kinase 1 (ADRBK1; SEQ ID NO:10), bladder cancer associated protein (BLCAP; SEQ ID NO:12), chromosome 22 open reading frame 5 (C22orf5; SEQ ID NO:14), CD81 antigen (target of antiproliferative antibody 1 (CD81; SEQ ID NO:16), CD9 antigen (p24) (CD9; SEQ ID NO:18), claudin 4 (CLDN4; SEQ ID NO:20), chloride intracellular channel 1 (CLIC1; SEQ ID NO:22), collagen, type VI, alpha 2 (COL6A2; SEQ ID NO:24), CTL2 (CTL2; SEQ ID NO:26), endothelin converting enzyme 1 (ECE1; SEQ ID NO:28), ephrin-B1 (EFNB1; SEQ ID NO:30), flotillin 2 (FLOT2; SEQ ID NO:32), intercellular adhesion molecule 3 (ICAM3; SEQ ID NO:34), iduronate 2-sulfatase (Hunter syndrome) (IDS; SEQ ID NO:36), jagged 2 (JAG2; SEQ ID NO:38), junctional adhesion molecule 1 (JAM1; SEQ ID NO:40), lectin, galactoside-binding soluble 3 binding protein (LGALS3BP; SEQ ID NO:42), similar to possible G-protein receptor (LOC146330; SEQ ID NO:44), CGI-78 protein (LOC51107; SEQ ID NO:46), lipoprotein lipase (LPL; SEQ ID NO:48), low density lipoprotein receptor-related protein 5 (LRP5; SEQ ID NO:50), Lutheran blood group (Auberger b antigen included) (LU; SEQ ID NO:52), membrane component, chromosome 11, surface marker 1 (M11S1; SEQ ID NO:54), serum constituent protein (MSE55; SEQ ID NO:56), neuropathy target esterase (NTE; SEQ ID NO:58), Homo sapiens cDNA FL31043 fis, clone HSYRA2000248 (PLEXIN A1) or Homo sapiens cDNA FLJ44113 fis, clone TESTI4046487, highly similar to Mus musculus plexin A1 (PLXNA1; SEQ ID NO:60), protein tyrosine phosphatase, receptor type, f polypeptide (PTPRF), interacting protein (liprin), alpha 3 (PPFIA3; SEQ ID NO:62), Homo sapiens peptide-histidine transporter 4 (PTR4), mRNA (PTR4; SEQ ID NO:64), solute carrier family 16 (moncarboxylic acid transporters) member 3 (SLC16A3; SEQ ID NO:66), solute carrier family 1 (neutral amino acid transporter) member 5 (SLC1A5; SEQ ID NO:68), solute carrier family 39 (zinc transporter) member 3 (SLC39A1; SEQ ID NO:70), serine protease inhibitor, Kunitz type 2 (SPINT2; SEQ ID NO:72), stanniocalcin 2 (STC2; SEQ ID NO:74), tumor necrosis receptor superfamily member 21 (TNFRSF21; SEQ ID NO:76), tumor rejection antigen (gp96) 1 (TRA1; SEQ ID NO:78), and transient receptor potential cation channel, subfamily M member 4 (TRPM4; SEQ ID NO:80). In any of the assays described herein, one can use a full-length gene, including a regulatory region of the gene, or a nucleic acid molecule encoding the gene product (protein encoded by the gene) or any fragment of such nucleic acid molecules, or any gene product or fragment thereof that is suitable for use in an assay to identify inhibitors of the target for the purpose of regulating apoptosis or inhibition of tumor growth, or to detect cancer in a patient sample.

In one embodiment of the invention, the down-regulation of the concentration or activity of a target gene or product by an inhibitor (including a GSE) depletes a cellular component required for protecting cells from apoptosis resulting in control of cell growth. In another embodiment of the invention, the down-regulation of the concentration or activity of one target gene or product by an inhibitor (including a GSE) depletes a cellular component that interacts with another gene or gene product required for protecting cells from apoptosis resulting in control of cell growth. In a preferred embodiment of the invention, the two genes are members of the same biological pathway and one gene or gene product regulates the expression or activity of the other gene or gene product. In another preferred embodiment of the invention, the two genes are members of the same biological pathway and the substrate of a protein encoded by one gene is a product of a biochemical reaction mediated by the protein encoded by the other gene. In still another preferred embodiment of the invention, the two genes are members of the same biological pathway and the product of a protein encoded by one gene is a substrate of a biochemical reaction mediated by the protein encoded by the other gene. In another embodiment, the two genes encode proteins that are isozymes of each other. In a preferred embodiment, at least one of the genes encodes an enzyme.

Target genes or proteins identified using GSEs can be further evaluated using a variety of methods to validate their involvement in cell growth, suppression of apoptosis and tumor formation. Such methods include methods that disrupt or “knock out” the expression of a target gene in a cell capable of apoptosis. Knock-out methods include somatic cell knock-outs and inhibitory RNA molecules including anti-sense oligonucleotides, siRNA molecules, RNAi molecules and RNA decoys. Target genes or proteins can also be evaluated by methods that include nucleic acid-based experiments such as Northern Blots, Real Time polymerase chain reaction or high density microarrays. Further evaluation can also be achieved using human/mouse xenograft models. For example, human tumor cells can be transfected with a GSE such that the GSE is expressed. Preferred tumor cells include HCT15, HT29, HCT116, SW480 and SW620 and MDA-MB-231 (e.g., see Examples). The transfected cells can then be implanted into mice, preferably nude mice. The growth of the tumor cells in the mouse can then be measured.

Once a gene has been identified as a potential target for supporting cell growth, assays can be used for associating a potential target with different tumor types. These assays include determining gene and protein expression of potential targets in different tumor cell types at different points of differentiation. Another assay can include determining the presence of a potential marker in patient samples using standard protein detection methods known to those of skill in the art. Targets that have been associated with cancer are also referred to as biomarkers. Preferred biomarkers of the present invention are listed in Table 1 (see Examples section).

Once one or more members of a biological pathway are identified as required for cell growth, the present invention can include identifying additional members of a biological pathway that are also required for cell growth. Such subsequent identification is within the skill of one in the art. GSEs, and therefore preferred targets of the present invention, are identified by selecting cells that exhibit certain hallmarks of apoptosis upon expression of the GSEs. Isolated GSEs are further prioritized based on their specificity for a neoplastic transformation state, such as their activity in transformed and non-transformed cells, and based on the p53 pathway status in cells expressing the GSEs. For example, GSEs can be prioritized by determining if the GSEs have activity in a p53 dependent and/or independent manner. GSEs specific for the neoplastic transformation state are preferred for identifying targets for anti-cancer drugs.

It will be understood that this invention is not limited to the particular methodology, protocols, cell lines, animal species or genera, constructs, or reagents described herein, as such may vary. It is also to be understood that the terminology used herein is for the purpose of describing particular embodiments only, and is not intended to limit the scope of the invention that will be limited only by the appended claims. All technical and scientific terms used herein have the same meaning as commonly understood to one of ordinary skill in the art to which this invention belongs unless clearly indicated otherwise.

As used herein, the term “isolated nucleic acid molecule” refers to a nucleic acid molecule that has been removed from its natural milieu (i.e., a molecule that has been subject to human manipulation) and can include DNA, RNA, or derivatives of either DNA or RNA. An isolated nucleic acid molecule can be isolated from its natural source or can be produced using recombinant DNA technology (e.g., polymerase chain reaction amplification) or chemical synthesis. Isolated nucleic acid molecules include natural nucleic acid molecules and homologs thereof, including, but not limited to, natural allelic variants and modified nucleic acid molecules in which nucleotides have been inserted, deleted, substituted, or inverted in such a manner that such modifications do not substantially interfere with the nucleic acid molecule's ability to control cell growth or encode a protein that controls cell growth.

It should also be appreciated that reference to an isolated nucleic acid molecule does not necessarily reflect the extent of purity of the nucleic acid molecule. Nucleic acid molecules can be isolated and obtained in substantial purity, generally as other than an intact chromosome. Usually, the nucleic acid molecule will be obtained substantially free of other nucleic acid sequences, generally being at least about 50%, and usually at least about 90% pure. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably.

According to the invention, reference to an “isolated nucleic acid molecule” refers to a nucleic acid molecule that is the size of or is smaller than a gene. Thus, an isolated nucleic acid molecule does not encompass isolated total genomic DNA or an isolated chromosome. As used herein, the term “gene” has the meaning that is well known in the art, that is, a nucleic acid sequence that includes the translated sequences that code for a protein (“exons”) and the untranslated intervening sequences (“introns”), and any regulatory elements necessary to transcribe and/or translate the protein. Included in the invention are nucleic acid molecules that are less than a full-length gene or less than a full-length coding sequence, such as fragments of a gene or coding sequence comprising, consisting essentially of, or consisting of, for example, a fragment of any of the nucleic acid sequences for target genes described in the present invention. A coding sequence can include genomic DNA without introns, cDNA or RNA that encodes a protein. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., are heterologous sequences).

In one embodiment, an isolated nucleic acid molecule useful in a method of the present invention is produced using recombinant DNA technology (e.g., polymerase chain reaction (PCR) amplification, cloning) or chemical synthesis. A nucleic acid molecule homologue can be produced using a number of methods known to those skilled in the art (see, for example, Sambrook et al., ibid.). For example, nucleic acid molecules can be modified using a variety of techniques including, but not limited to, classical mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

The term isolated nucleic acid molecule does not necessarily connote any specific minimum length unless set forth by reference to a minimum number of nucleotides or by a function of the nucleic acid molecule. The minimum size of a nucleic acid molecule of the present invention is generally a size sufficient to encode a protein having the desired biological activity, a size sufficient to inhibit the expression and/or activity of a target as described herein (e.g., as in a GSE), a size sufficient for use in a screening assay or diagnostic method of the invention, or a size sufficient to form a probe or oligonucleotide primer that is capable of forming a stable hybrid with the complementary sequence of a nucleic acid molecule. As such, the size of a nucleic acid molecule of the present invention can be dependent on nucleic acid composition and percent homology or identity between the nucleic acid molecule and complementary sequence as well as upon hybridization conditions per se (e.g., temperature, salt concentration, and formamide concentration) and the intended use of the nucleic acid molecule. The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a fragment of a gene, a portion of a protein encoding sequence, or a nucleic acid sequence encoding a full-length protein (including a complete gene).

Some embodiments of the present invention may include the production and/or use of a recombinant nucleic acid molecule comprising a recombinant vector and a nucleic acid molecule comprising a nucleic acid sequence encoding a gene or fragment thereof as described herein. According to the present invention, a recombinant vector is an engineered (i.e., artificially produced) nucleic acid molecule that is used as a tool for manipulating a nucleic acid sequence of choice and for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid molecules of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant organism (e.g., a microbe or a plant). The entire vector can remain in place within a host cell, or under certain conditions, the plasmid DNA can be deleted, leaving behind the nucleic acid molecule of the present invention. The integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced is inserted into the recombinant vector to produce a recombinant nucleic acid molecule. The nucleic acid sequence encoding the protein to be produced is inserted into the vector in a manner that operatively links the nucleic acid sequence to regulatory sequences in the vector that enable the transcription and translation of the nucleic acid sequence within the recombinant host cell.

In another embodiment, a recombinant vector used in a recombinant nucleic acid molecule of the present invention is a targeting vector. As used herein, the phrase “targeting vector” is used to refer to a vector that is used to deliver a particular nucleic acid molecule into a recombinant host cell, wherein the nucleic acid molecule is used to delete or inactivate an endogenous gene within the host cell or microorganism (i.e., used for targeted gene disruption or knock-out technology). Such a vector may also be known in the art as a “knock-out” vector. In one aspect of this embodiment, a portion of the vector, but more typically, the nucleic acid molecule inserted into the vector (i.e., the insert), has a nucleic acid sequence that is homologous to a nucleic acid sequence of a target gene in the host cell (i.e., a gene which is targeted to be deleted or inactivated). The nucleic acid sequence of the vector insert is designed to bind to the target gene such that the target gene and the insert undergo homologous recombination, whereby the endogenous target gene is deleted, inactivated or attenuated (i.e., by at least a portion of the endogenous target gene being mutated or deleted).

Typically, a recombinant nucleic acid molecule includes at least one nucleic acid molecule of the present invention operatively linked to one or more expression control sequences, including transcription control sequences and translation control sequences. As used herein, the phrase “recombinant molecule” or “recombinant nucleic acid molecule” primarily refers to a nucleic acid molecule or nucleic acid sequence operatively linked to an expression control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence (e.g., a transcription control sequence and/or a translation control sequence) in a manner such that the molecule is able to be expressed when transfected (i.e., transformed, transduced, transfected, conjugated or conduced) into a host cell. Transcription control sequences are sequences that control the initiation, elongation, or termination of transcription. Particularly important transcription control sequences are those that control transcription initiation, such as promoter, enhancer, operator and repressor sequences. Suitable transcription control sequences include any transcription control sequence that can function in a host cell or organism into which the recombinant nucleic acid molecule is to be introduced.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells. In microbial systems, the term “transformation” is used to describe an inherited change due to the acquisition of exogenous nucleic acids by the microorganism and is essentially synonymous with the term “transfection.” However, in animal cells, transformation has acquired a second meaning that can refer to changes in the growth properties of cells in culture (described above) after they become cancerous, for example. Therefore, to avoid confusion, the term “transfection” is preferably used with regard to the introduction of exogenous nucleic acids into animal cells, including human cells, and is used herein to generally encompass transfection of animal cells and transformation of microbial cells, to the extent that the terms pertain to the introduction of exogenous nucleic acids into a cell. Therefore, transfection techniques include, but are not limited to, transformation, chemical treatment of cells, particle bombardment, electroporation, microinjection, lipofection, adsorption, infection and protoplast fusion.

A recombinant cell is preferably produced by transforming a host cell with one or more recombinant molecules, each comprising one or more nucleic acid molecules operatively linked to an expression vector containing one or more expression control sequences.

“Hybridization” has the meaning that is well known in the art, that is, the formation of a duplex structure by two single-stranded nucleic acids due to complementary base pairing. Hybridization can occur between exactly complementary nucleic acid strands or between nucleic acid strands that contain some regions of mismatch. As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety. “Stringent hybridization” has a meaning well-established in the art, that is, hybridization performed at a salt concentration of no more than 1M and a temperature of at least 25 degrees Celsius. For example, conditions of 5×SSPE (750 mM NaCl, 50 mM Sodium Phosphate, 5 mM EDTA, pH 7.4) and a temperature of 55 degrees to 60 degrees Celsius are suitable. For example, in one embodiment, “moderately stringent conditions” can be defined as hybridizations carried out as described above, followed by washing in 0.2×SSC and 0.1% SDS at 42 degrees Celsius (Ausubel et al., 1989, Current Protocols for Molecular Biology, ibid.).

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (low stringency), more preferably, between about 28° C. and about 42° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

In one embodiment of the present invention, any amino acid sequence described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

As discussed above, one embodiment of the present invention relates to methods for identifying compounds that induce or increase or upregulate apoptosis in a cell by inhibiting genes or gene products involved in the control of cell growth. Once a gene has been identified as a target for supporting cell growth, an assay can be used for screening and selecting a chemical compound or a biological compound having activity as an anti-tumor therapeutic based on the ability of the compound to down-regulate expression of the gene or inhibit activity of its gene product. Reference herein to inhibiting a target, can refer to one or both of inhibiting expression of a target gene and inhibiting the translation and/or activity of its corresponding expression product. Such a compound can be referred to herein as therapeutic compound. For example, a cell line that naturally expresses the gene of interest or has been transfected with the gene or other recombinant nucleic acid molecule encoding the protein of interest is incubated with various compounds, also referred to as candidate compounds, test compounds, or putative regulatory compounds. A reduction of the expression of the gene of interest or an inhibition of the activities of its encoded product (e.g., biological activity, which can include the involvement of the protein in the protection of the cell from apoptotic processes) may be used to identify a therapeutic compound. Therapeutic compounds identified in this manner can then be re-tested, if desired, in other assays to confirm their activities against cellular apoptotic processes.

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications, activities or interactions which result in a decrease in protein expression or a decrease in the activity of the protein, can be referred to as inactivation (complete or partial), down-regulation, reduced action, or decreased action or activity of a protein. Similarly, modifications, activities or interactions which result in an increase in protein expression or an increase in the activity of the protein, can be referred to as amplification, overproduction, activation, enhancement, up-regulation or increased action of a protein. The biological activity of a protein according to the invention can be measured or evaluated using any assay for the biological activity of the protein as known in the art. Such assays can include, but are not limited to, binding assays, assays to determine internalization of the protein and/or associated proteins, enzyme assays, cell signal transduction assays (e.g., phosphorylation assays), and/or assays for determining downstream cellular events that result from activation or binding of the cell surface protein (e.g., expression of downstream genes, production of various biological mediators, etc.). The assay can also measure the ability of the protein to contribute to the regulation of apoptosis in a cell. Such assays are described in detail herein. According to the present invention, a biologically active fragment or homologue of a gene or protein maintains the ability to be useful in a method of the present invention. Therefore, the biologically active fragment or homologue maintains the ability to be used to identify regulators (e.g., inhibitors) of the native gene or protein when, for example, the biologically active fragment or homologue is expressed by a cell. Therefore, the biologically active fragment or homologue has a structure that is sufficiently similar to the structure of the native gene or protein that a regulatory compound can be identified by its ability to bind to and/or regulate the expression or activity of the fragment or homologue in a manner consistent with the regulation of the native gene or protein.

Compounds to be screened in the methods of the invention include known organic compounds such as antibodies, products of peptide libraries, and products of chemical combinatorial libraries. Compounds may also be identified using rational drug design relying on the structure of the product of a gene. Such methods are known to those of skill in the art and involve the use of three-dimensional imaging software programs. For example, various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

As used herein, a mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

As used herein, the term “test compound”, “putative inhibitory compound” or “putative regulatory compound” refers to compounds having an unknown or previously unappreciated regulatory activity in a particular process. As such, the term “identify” with regard to methods to identify compounds is intended to include all compounds, the usefulness of which as a regulatory compound for the purposes of inhibiting cell growth is determined by a method of the present invention.

In one embodiment of the invention, inhibitors of cell growth are identified by exposing a target gene to a test compound; measuring the expression of a target; and selecting a compound that down-regulates (reduces, decreases, inhibits, blocks) the expression of the target. For example, the putative inhibitor can be exposed to a cell that expresses the target gene (endogenously or recombinantly). A preferred cell to use in an assay includes a mammalian cell that either naturally expresses the target gene or has been transformed with a recombinant form of the target gene, such as a recombinant nucleic acid molecule comprising a nucleic acid sequence encoding the target protein or a useful fragment thereof. Methods to determine expression levels of a gene are well known in the art.

The conditions under which a cell, cell lysate, nucleic acid molecule or protein of the present invention is exposed to or contacted with a putative regulatory compound, such as by mixing, are any suitable culture or assay conditions. In the case of a cell-based assay, the conditions include an effective medium in which the cell can be cultured or in which the cell lysate can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. A preferred amount of putative regulatory compound(s) can comprise between about 1 nM to about 10 mM of putative regulatory compound(s) per well of a 96-well plate.

As used herein, the term “expression”, when used in connection with detecting the expression of a target of the present invention, can refer to detecting transcription of the target gene and/or to detecting translation of the target protein encoded by the target gene. To detect expression of a target refers to the act of actively determining whether a target is expressed or not. This can include determining whether the target expression is upregulated as compared to a control, downregulated as compared to a control, or unchanged as compared to a control. Therefore, the step of detecting expression does not require that expression of the target actually is upregulated or downregulated, but rather, can also include detecting that the expression of the target has not changed (i.e., detecting no expression of the target or no change in expression of the target). Expression of transcripts and/or proteins is measured by any of a variety of known methods in the art. For RNA expression, methods include but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

In a preferred embodiment, the expression of the target gene is measured by the polymerase chain reaction. In another embodiment, the expression of the target gene is measured using polyacrylamide gel analysis, chromatography or spectroscopy.

In another preferred embodiment, the expression of the target gene is measured by measuring the production of the encoded protein (measuring translation of the protein). Measurement of translation of a protein includes any suitable method for detecting and/or measuring proteins from a cell or cell extract. Such methods include, but are not limited to, immunoblot (e.g., Western blot), enzyme-linked immunosorbant assay (ELISA), radioimmunoassay (RIA), immunoprecipitation, immunohistochemistry, immunofluorescence, fluorescence activated cell sorting (FACS) and immunofluorescence microscopy. Particularly preferred methods for detection of proteins include any single-cell assay, including immunohistochemistry and immunofluorescence assays. For example, one can use a detection agent, such as an antibody that specifically recognizes (selectively binds to) the protein encoded by the gene. Such methods are well known in the art.

Designing a compound for testing in a method of the present invention can include creating a new chemical compound or searching databases of libraries of known compounds (e.g., a compound listed in a computational screening database containing three dimensional structures of known compounds). Designing can also be performed by simulating chemical compounds having substitute moieties at certain structural features. The step of designing can include selecting a chemical compound based on a known function of the compound. A preferred step of designing comprises computational screening of one or more databases of compounds in which the three dimensional structure of the compound is known and is interacted (e.g., docked, aligned, matched, interfaced) with the three dimensional structure of a target by computer (e.g. as described by Humblet and Dunbar, Animal Reports in Medicinal Chemistry, vol. 28, pp. 275-283, 1993, M Venuti, ed., Academic Press). Methods to synthesize suitable chemical compounds are known to those of skill in the art and depend upon the structure of the chemical being synthesized. Methods to evaluate the bioactivity of the synthesized compound depend upon the bioactivity of the compound (e.g., inhibitory or stimulatory).

Accordingly, in another embodiment of the invention, therapeutic compounds can be selected by determining the three-dimensional structure of a target; and determining or designing the three-dimensional structure of a therapeutic or regulatory compound by rational drug design or detecting a structure that interacts with the target structure from a library of known compound structures. Preferably, the structure of the therapeutic compound is determined using computer software capable of modeling the interaction of a therapeutic compound with the target. One of skill in the art can select the appropriate three-dimensional structure, therapeutic or regulatory compound, and analytical software based on the identity of the target.

For example, suitable candidate chemical compounds can align to a subset of residues described for a target site. Preferably, a candidate chemical compound comprises a conformation that promotes the formation of covalent or noncovalent crosslinking between the target site and the candidate chemical compound. Preferably, a candidate chemical compound binds to a surface adjacent to a target site to provide an additional site of interaction in a complex. When designing an antagonist, for example, the antagonist should bind with sufficient affinity to the binding site or to substantially prohibit a ligand (i.e., a molecule that specifically binds to the target site) from binding to a target area. It will be appreciated by one of skill in the art that it is not necessary that the complementarity between a candidate chemical compound and a target site extend over all residues specified here in order to inhibit or promote binding of a ligand.

In general, the design of a chemical compound possessing stereochemical complementarity can be accomplished by techniques that optimize, chemically or geometrically, the “fit” between a chemical compound and a target site. Such techniques are disclosed by, for example, Sheridan and Venkataraghavan, Acc. Chem Res., vol. 20, p. 322, 1987: Goodford, J Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

As another example, a “geometric approach” is used. In a geometric approach, the number of internal degrees of freedom (and the corresponding local minima in the molecular conformation space) is reduced by considering only the geometric (hard sphere) interactions of two rigid bodies, where one body (the active site) contains “pockets” or “grooves” that form binding sites for the second body (the complementing molecule, such as a ligand). The geometric approach is described by Kuntz et al., J. Mol. Biol., vol. 161, p. 269, 1982, which is incorporated by this reference herein in its entirety. The algorithm for chemical compound design can be implemented using the software program DOCK Package, Version 1.0 (available from the Regents of the University of California). Pursuant to the Kuntz algorithm, the shape of the cavity or groove on the surface of a structure at a binding site or interface is defined as a series of overlapping spheres of different radii. One or more extant databases of crystallographic data (e.g., the Cambridge Structural Database System maintained by University Chemical Laboratory, Cambridge University, Lensfield Road, Cambridge CB2 1EW, U.K.) or the Protein Data Bank maintained by Brookhaven National Laboratory, is then searched for chemical compounds that approximate the shape thus defined. Chemical compounds identified by the geometric approach can be modified to satisfy criteria associated with chemical complementarity, such as hydrogen bonding, ionic interactions or Van der Waals interactions.

As yet another example, one can determine the interaction of chemical groups (“probes”) with an active site at sample positions within and around a binding site or interface, resulting in an array of energy values from which three dimensional contour surfaces at selected energy levels can be generated. This method is referred to herein as a “chemical-probe approach.” The chemical-probe approach to the design of a chemical compound useful of the present invention is described by, for example, Goodford, J. Med. Chem., vol. 28, p. 849, 1985, which is incorporated by this reference herein in its entirety, and is implemented using an appropriate software package, including for example, GRID (available from Molecular Discovery Ltd., Oxford OX29LL, U.K.). The chemical prerequisites for a site-complementing molecule can be identified at the outset, by probing the active site of a protein with different chemical probes, e.g., water, a methyl group, an amine nitrogen, a carboxyl oxygen and/or a hydroxyl. Preferred sites for interaction between an active site and a probe are determined. Putative complementary chemical compounds can be generated using the resulting three dimensional patterns of such sites.

Candidate compounds identified or designed by the above-described methods can be synthesized using techniques known in the art, and depending on the type of compound. Synthesis techniques for the production of non-protein compounds, including organic and inorganic compounds are well known in the art. For example, for smaller peptides, chemical synthesis methods are preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289: 3-13; Wade et al., 1993, Australas Biotechnol. 3(6): 332-336; Wong et al., 1991, Experientia 47(11-12):1123-1129; Carey et al., 1991, Ciba Found Symp. 158: 187-203; Plaue et al., 1990, Biologicals 18(3): 147-157; Bodanszky, 1985, Int. J. Pept. Protein Res. 25(5): 449-474; or H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture. A compound that is a protein or peptide can also be produced using recombinant DNA technology and methods standard in the art, particularly if larger quantities of a protein are desired.

In still another embodiment of the invention, inhibitors of cell growth are identified by exposing a target to a candidate compound; measuring the binding of the candidate compound to the target; and selecting a compound that binds to the target at a desired concentration, affinity, or avidity. In a preferred embodiment, the assay is performed under conditions conducive to promoting the interaction or binding of the compound to the target. One of skill in the art can determine such conditions based on the target and the compound being used in the assay. In one embodiment, a BIAcore machine can be used to determine the binding constant of a complex between the target protein (a protein encoded by the target gene) and a natural ligand in the presence and absence of the candidate compound. For example, the target protein or a ligand binding fragment thereof can be immobilized on a substrate. A natural or synthetic ligand is contacted with the substrate to form a complex. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Contacting a candidate compound at various concentrations with the complex and monitoring the response function (e.g., the change in the refractive index with respect to time) allows the complex dissociation constant to be determined in the presence of the test compound and indicates whether the candidate compound is either an inhibitor or an agonist of the complex. Alternatively, the candidate compound can be contacted with the immobilized target protein at the same time as the ligand to see if the candidate compound inhibits or stabilizes the binding of the ligand to the target protein.

Other suitable assays for measuring the binding of a candidate compound to a target protein or for measuring the ability of a candidate compound to affect the binding of the target protein to another protein or molecule include, but are not limited to, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry. Other assays include those that are suitable for monitoring the effects of protein binding, including, but not limited to, cell-based assays such as: cytokine secretion assays, or intracellular signal transduction assays that determine, for example, protein or lipid phosphorylation, mediator release or intracellular Ca⁺⁺ mobilization.

In yet another embodiment, inhibitors of cellular growth are identified by exposing a target protein of the present invention (or a cell expressing the protein naturally or recombinantly) to a candidate compound and measuring the ability of the compound to inhibit (reduce, decrease, block) a biological activity of the protein. In one embodiment, the biological activity of a protein encoded by the target gene is measured by measuring the amount of product generated in a biochemical reaction mediated by the protein encoded by the target gene. In still another embodiment, the activity of the protein encoded by the target gene is measured by measuring the amount of substrate generated in a biochemical reaction mediated by the protein encoded by the target gene. In another embodiment, a biological activity is measured by measuring a specific event in a cell-based assay, such as release or secretion of a biological mediator or compound that is regulated by the activity of the target protein, measuring intracellular signal transduction assays that determine, for example, protein or lipid phosphorylation, mediator release or intracellular Ca⁺⁺ mobilization. Preferably, the activity of the protein is measured in the presence and absence of the candidate compound, or in the presence of another suitable control compound.

In one embodiment of the invention, when the protein encoded by a target gene is an enzyme, a therapeutic compound is identified by exposing the enzyme encoded by a target gene to a test compound; measuring the activity of the enzyme encoded by the target gene in the presence and absence of the compound; and selecting a compound that down-regulates or inhibits the activity of the enzyme encoded by the target gene. Methods to measure enzymatic activity are well known to those skilled in the art and are selected based on the identity of the enzyme being tested. For example, if the enzyme is a kinase, phosphorylation assays can be used.

In addition to methods for identifying and producing a biological compound that inhibits cell growth, the present invention includes methods known in the art that down-regulate expression or function of a target gene. For example, antisense RNA and DNA molecules may be used to directly block translation of mRNA encoded by these genes by binding to targeted mRNA and preventing protein translation. Polydeoxyribonucleotides can form sequence-specific triple helices by hydrogen bonding to specific complementary sequences in duplexed DNA to effect specific down-regulation of target gene expression. Formation of specific triple helices may selectively inhibit the replication or expression of a target gene by prohibiting the specific binding of functional trans-acting factors.

Ribozymes are enzymatic RNA molecules capable of catalyzing the specific cleavage of RNA. Ribozyme action involves sequence specific hybridization of the ribozyme molecule to complementary target RNA, followed by endonucleolytic cleavage. Within the scope of the invention are ribozyme embodiments including engineered hammerhead motif ribozyme molecules that specifically and efficiently catalyze endonucleolytic cleavage of RNA sequences. Antisense RNA molecules showing high-affinity binding to target sequences can also be used as ribozymes by addition of enzymatically active sequences known to those skilled in the art.

Polynucleotides to be used in triplex helix formation should be single-stranded and composed of deoxynucleotides. The base composition of these polynucleotides must be designed to promote triple helix formation via Hoogsteen base pairing rules, which generally require sizeable stretches of either purines or pyrimidines to be present on one strand of a duplex. Polynucleotide sequences may be pyrimidine-based, which will result in TAT and CGC triplets across the three associated strands of the resulting triple helix. The pyrimidine-rich polynucleotides provide base complementarity to a purine-rich region of a single strand of the duplex in a parallel orientation to that strand. In addition, polynucleotides may be chosen that are purine-rich, for example, containing a stretch of G residues. These polynucleotides will form a triple helix with a DNA duplex that is rich in GC pairs, in which the majority of the purine residues are located on a single strand of the targeted duplex, resulting in GGC triplets across the three strands in the triplex.

Alternatively, sequences that can be targeted for triple helix formation can be increased by creating a so-called “switchback” polynucleotide. Switchback polynucleotides are synthesized in an alternating 5′-3′, 3′-5′ manner, so that they base pair with first one strand of a duplex and then the other, eliminating the necessity for a sizeable stretch of either purines or pyrimidines to be present on one strand of a duplex.

Both antisense RNA and DNA molecules, and ribozymes of the invention may be prepared by any method known in the art. These include techniques for chemically synthesizing polynucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into host cells.

Various modifications to the nucleic acid molecules may be introduced as a means of increasing intracellular stability and half-life. Possible modifications include, but are not limited to, the addition of flanking sequences of ribonucleotides or deoxyribonucleotides to the 5′ or 3′ ends of the molecule or the use of phosphorothioate or 2′ O-methyl rather than phosphodiesterase linkages within the oligodeoxyribonucleotide backbone.

Preferably, methods used to identify therapeutic compounds are customized for each target gene or product. If the target product is an enzyme, then the enzyme will be expressed in cell culture and purified. The enzyme will then be screened in vitro against therapeutic compounds to look for inhibition of that enzymatic activity. If the target is a non-catalytic protein, then it will also be expressed and purified. Therapeutic compounds will then be tested for their ability to prevent, for example, the binding of a site-specific antibody or a target-specific ligand to the target product.

In a preferred embodiment, therapeutic compounds that bind to target products are identified, then those compounds can be further tested in biological assays that test for characteristics such as apoptosis, tumor suppressor status (e.g., p53 status), tumor cell growth and any other customary measure of anti-cancer activity.

In one embodiment of the invention, a therapeutic compound is not toxic to a human host cell. In another embodiment, the therapeutic compound is cytostatic or cytotoxic.

In one embodiment of the invention, a pharmaceutical composition is prepared from a therapeutically-effective amount of a therapeutic compound of the invention and a pharmaceutically-acceptable carrier. Pharmaceutically-acceptable carriers are well known to those with skill in the art. The pharmaceutical compositions of the present invention can be manufactured in a manner that is itself known, e.g., by means of a conventional mixing, dissolving, granulating, dragee-making, levigating, emulsifying, encapsulating, entrapping or lyophilizing processes. As used herein, a pharmaceutically acceptable carrier refers to any substance suitable for delivering a therapeutic composition useful in the method of the present invention to a suitable in vivo or ex vivo site. Pharmaceutical compositions for use in accordance with the present invention thus can be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art. For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. The compounds can be formulated for parenteral administration by injection, e.g., by bolus injection or continuous infusion. Formulations for injection can be presented in unit dosage form, e.g., in ampoules or in multi-dose containers, with an added preservative. The compounds can also be formulated in rectal compositions such as suppositories or retention enemas, e.g., containing conventional suppository bases such as cocoa butter or other glycerides.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

According to the present invention, an effective administration protocol (i.e., administering a composition of the present invention in an effective manner) comprises suitable dose parameters and modes of administration that result in delivery of the compound or composition to a patient or to a target site, cell or tissue in the patient, and subsequent inhibition of the growth of the target cell, preferably so that the patient obtains some measurable, observable or perceived benefit from such administration. In some situations, where the target cell population is accessible for sampling, effective dose parameters can be determined using methods as described herein for assessment of tumor growth. Such methods include removing a sample of the target cell population from the patient prior to and after the compound or composition is administered, and measuring changes expression or biological activity of a target, as well as measuring inhibition of the growth of the cell. Alternatively, effective dose parameters can be determined by experimentation using in vitro cell cultures, in vivo animal models, and eventually, clinical trials if the patient is human. Effective dose parameters can be determined using methods standard in the art. Such methods include, for example, determination of survival rates, side effects (i.e., toxicity) and progression or regression of disease. Compounds which exhibit high therapeutic indices are preferred. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

Dosage amount and interval can be adjusted individually to provide plasma levels of the active moiety which are sufficient to maintain the inhibitory effects. Usual patient dosages for systemic administration range from 100-2000 mg/day. Stated in terms of patient body surface areas, usual dosages range from 50-910 mg/m²/day. Usual average plasma levels should be maintained within 0.1-1000 μM. In cases of local administration or selective uptake, the effective local concentration of the compound can not be related to plasma concentration.

The amount of composition administered will, of course, be dependent on the subject being treated, on the subject's body surface area, the severity of the affliction, the manner of administration and the judgment of the prescribing physician.

Suitable routes of administration can, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections. Alternatively, one can administer the compound in a local rather than systemic manner, for example, via injection of the compound directly into a specific tissue, often in a depot or sustained release formulation. Furthermore, one can administer the compound in a targeted drug delivery system, for example, in a liposome and/or conjugated with a cell-specific antibody. The liposomes and cell-specific antibody will be targeted to and taken up selectively by tumor cells.

Accordingly, a further embodiment of the invention is a method for inducing apoptosis in a cell by inhibiting a target of the present invention, i.e., a target selected from the group consisting of any of the targets listed in Table I and/or represented by any of SEQ ID NOs:1-80. For example, this method can be conducted in vivo by administering to an individual an inhibitory or therapeutic compound as generally discussed herein. In addition, the method can be conducted in vitro or ex vivo.

A further embodiment of the present invention is a method for the diagnosis of a tumor or the monitoring of a tumor growth or regression or a tumor therapy in a patient. The methods include determining the level of a marker (also referred to as a biomarker) in a patient sample, wherein the marker is selected from any of the biomarkers listed in Table 1 or represented by any of SEQ ID NOs:1-80.

The first step of this method of the present invention includes detecting the expression or biological activity of a biomarker in a test sample from a patient (also called a patient sample). Suitable methods of obtaining a patient sample are known to a person of skill in the art. A patient sample can include any bodily fluid or tissue from a patient that may contain tumor cells or proteins of tumor cells. More specifically, according to the present invention, the term “test sample” or “patient sample” can be used generally to refer to a sample of any type which contains cells or products that have been secreted from cells to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid.

A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, contains the cells to be evaluated for marker expression or biological activity and/or may contain a soluble biomarker that is secreted by cells, and is a fluid obtained by any method suitable for the particular bodily fluid to be sampled. Bodily fluids suitable for sampling include, but are not limited to, blood, mucous, seminal fluid, saliva, breast milk, bile and urine.

In general, the sample type (i.e., cell, tissue or bodily fluid) is selected based on the accessibility and structure of the organ or tissue to be evaluated for tumor cell growth and/or on what type of cancer is to be evaluated. For example, if the organ/tissue to be evaluated is the breast, the sample can be a sample of epithelial cells from a biopsy (i.e., a cell sample) or a breast tissue sample from a biopsy (a tissue sample). The sample that is most useful in the present invention will be cells, tissues or bodily fluids isolated from a patient by a biopsy or surgery or routine laboratory fluid collection.

Once a sample is obtained from the patient, the sample is evaluated for detection of the expression or biological activity of the biomarker of the present invention in the cells of the sample. Expression and biological activity of biomarkers of the invention and methods of detecting or measuring the same have been described in detail above with regard to the description of the use of the biomarkers as targets.

For example, the level of the marker can be determined by conventional methods such as expression assays to determine the level of expression of the gene, by biochemical assays to determine the level of the gene product, or by immunoassays. If appropriate, the marker can be identified as a cell surface molecule in tissue or in a bodily fluid, such as serum. For example, a patient sample, which can be immobilized, can be contacted with an antibody, or an antibody fragment, that selectively binds to the marker and determining whether the anti-marker antibody or fragment thereof has bound to the marker. As used herein, the term “selectively binds to” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry. In a particular immunoassay, the marker level is determined using a first monoclonal antibody that binds specifically to the marker and a second antibody that binds to the first antibody.

In one embodiment, the amino acid sequence of a biomarker or the nucleic acid sequence of the corresponding gene can be used as a basis for detection. For example, detection can refer to detection of gene expression by determining the concentration of messenger RNA using common methods such as northern blot analysis, gene chip array analysis, Taqman analysis or other DNA/RNA hybridization platforms. The over or under expression of a biomarker can be an indication of the presence of a tumor or the predisposition for such tumor. Expression can be compared in patient samples versus samples isolated from healthy individuals.

In one embodiment of the method of the present invention, the level of a biomarker of the present invention is determined by determining the protein level of that biomarker in tissue. Suitable tissue tissues include tumor tissue and cell material obtained by biopsy.

In another embodiment of the method of the present invention, the level of a biomarker of the present invention is determined by determining a soluble form of a biomarker in a bodily fluid. Suitable bodily fluids include serum, ascitic or pleural fluid, serum being preferred. Levels of biomarker can be determined using various methods known in the art, including antibody binding assays, mass spectrometry analysis, 2-dimensional gel analysis and other methods used to quantify the presence of protein in solution. One preferred method of the present invention is to immobilize a biomarker to a solid substrate and then incubate the biomarker with a patient's serum. Bound antibodies to the biomarker are then detected by means of an enzyme-conjugated second antibody and a color reaction. Another preferred method is to immobilize an antibody that binds to a biomarker to a solid substrate and incubate the antibody with patient serum. Biomarker in the serum binds to the immobilized antibody and is detected using a second different antibody that binds to the biomarker and a color reaction. Another preferred method of the present invention is to contact an antibody that binds to a biomarker with a patient sample and then determining whether the antibody has been bound to the biomarker. Such method can be achieved using known methods including fluorescence cell sorter (FACS) analysis.

Suitable detection methods of a biomarker, an antibody that binds to a biomarker, or suitable nucleic acid probes, are known to those of skill in the art. The detection of biomarkers using antibodies is preferred, the same antibody being useful for both the soluble form and the form on the cell surface. Suitable antibodies for the method of the present invention include monoclonal antibodies, polyclonal antibodies, and fragments thereof. The antibody fragment refers to all parts of the antibody that bind to the biomarker including Fab, Fv or single-chain Fv fragments. Methods to produce such fragments are known to those of skill in the art. Preferred antibodies include monoclonal antibodies. Such antibodies can be produced using standard methods in the art.

Another method of the present invention can include immobilizing patient tissue in, for example, paraffin. The immobilized tissue can be sectioned and then contacted with an antibody that binds to a biomarker.

In the diagnostic/prognostic methods of the invention, if the level of the marker is greater than a normal level, the level of the marker is considered to be indicative of the presence of tumor cells. A normal level can be determined in a variety of ways. For example, if a patient history is known, a baseline level of the marker can be determined and higher levels will be indicative of tumor cells. Alternatively, a normal level can be based on the level for a healthy (i.e., without tumor) individual in a given population. That is, a normal level can be based on a population having similar characteristics (e.g., age, sex, race, medical history) as the patient in question.

More specifically, according to the present invention, a “baseline level” is a control level, and in some embodiments (but not all embodiments, depending on the method), a normal level, of biomarker expression or activity against which a test level of biomarker expression or biological activity (i.e., in the test sample) can be compared. Therefore, it can be determined, based on the control or baseline level of biomarker expression or biological activity, whether a sample to be evaluated for tumor cell growth has a measurable increase, decrease, or substantially no change in biomarker expression or biological activity, as compared to the baseline level. In one aspect, the baseline level can be indicative of the cell growth expected in a normal (i.e., healthy, negative control, non-tumor) cell sample. Therefore, the term “negative control” used in reference to a baseline level of biomarker expression or biological activity typically refers to a baseline level established in a sample from the patient or from a population of individuals which is believed to be normal (i.e., non-tumorous, not undergoing neoplastic transformation, not exhibiting inappropriate cell growth). It is noted that the “negative control” most typically has a lower level of biomarker expression or activity than would be detected in an experimental cell having inappropriate, increased cell growth, because the expression/biological activity of the biomarkers described herein are correlated with cell growth in most tumor cell types. In another embodiment, a baseline can be indicative of a positive diagnosis of tumor cell growth. Such a baseline level, also referred to herein as a “positive control” baseline, refers to a level of biomarker expression or biological activity established in a cell sample from the patient, another patient, or a population of individuals, wherein the sample was believed, based on data for that cell sample, to be neoplastically transformed (i.e., tumorous, exhibiting inappropriate cell growth, cancerous). It is noted that this “positive control” will most typically have a higher level of biomarker expression or activity than in a normal cell, again due to the correlative relationship between the biomarkers of the present invention and cell growth in the majority of tumor cells. In yet another embodiment, the baseline level can be established from a previous sample from the patient being tested, so that the tumor growth of a patient can be monitored over time and/or so that the efficacy of a given therapeutic protocol can be evaluated over time. Methods for detecting biomarker expression or biological activity are described in detail above.

The method for establishing a baseline level of biomarker expression or activity is selected based on the sample type, the tissue or organ from which the sample is obtained, the status of the patient to be evaluated, and, as discussed above, the focus or goal of the assay (e.g., diagnosis, staging, monitoring). Preferably, the method is the same method that will be used to evaluate the sample in the patient. In a most preferred embodiment, the baseline level is established using the same cell type as the cell to be evaluated.

In one embodiment, the baseline level of biomarker expression or biological activity is established in an autologous control sample obtained from the patient. The autologous control sample can be a sample of isolated cells, a tissue sample or a bodily fluid sample, and is preferably a cell sample or tissue sample. According to the present invention, and as used in the art, the term “autologous” means that the sample is obtained from the same patient from which the sample to be evaluated is obtained. The control sample should be of or from the same cell type and preferably, the control sample is obtained from the same organ, tissue or bodily fluid as the sample to be evaluated, such that the control sample serves as the best possible baseline for the sample to be evaluated. In one embodiment, when the goal of the assay is diagnosis of abnormal cell growth, it is desirable to take the control sample from a population of cells, a tissue or a bodily fluid which is believed to represent a “normal” cell, tissue, or bodily fluid, or at a minimum, a cell or tissue which is least likely to be undergoing or potentially be predisposed to develop tumor cell growth. For example, if the sample to be evaluated is an area of apparently abnormal cell growth, such as a tumorous mass, the control sample is preferably obtained from a section of apparently normal tissue (i.e., an area other than and preferably a reasonable distance from the tumorous mass) in the tissue or organ where the tumorous mass is growing. In one aspect, if a tumor to be evaluated is in the colon, the test sample would be obtained from the suspected tumor mass and the control sample would be obtained from a different section of the colon, which is separate from the area where the mass is located and which does not show signs of uncontrolled cellular proliferation.

In another embodiment, when the goal is to monitor tumor cell growth in the patient, the autologous baseline sample is typically a previous sample from the patient which was taken from an apparent or confirmed tumorous mass, and/or from apparently normal (i.e., non-tumor) tissue in the patient (or a different type of baseline for normal can be used, as discussed below).

Therefore, a second method for establishing a baseline level of biomarker expression or biological activity is to establish a baseline level of biomarker expression or biological activity from at least one measurement of biomarker expression or biological activity in a previous sample from the same patient. Such a sample is also an autologous sample, but is taken from the patient at a different time point than the sample to be tested. Preferably, the previous sample(s) were of a same cell type, tissue type or bodily fluid type as the sample to be presently evaluated. In one embodiment, the previous sample resulted in a negative diagnosis (i.e., no tumor cell growth, or potential therefore, was identified). In this embodiment, a new sample is evaluated periodically (e.g., at annual physicals), and as long as the patient is determined to be negative for tumor development, an average or other suitable statistically appropriate baseline of the previous samples can be used as a “negative control” for subsequent evaluations. For the first evaluation, an alternate control can be used, as described below, or additional testing may be performed to confirm an initial negative diagnosis, if desired, and the value for biomarker expression or biological activity can be used thereafter. This type of baseline control is frequently used in other clinical diagnosis procedures where a “normal” level may differ from patient to patient and/or where obtaining an autologous control sample at the time of diagnosis is not possible, not practical or not beneficial. For example, for a patient who has periodic mammograms, the previous mammograms serve as baseline controls for the mammary tissue of the individual patient. Similarly, for a patient who is regularly screened for prostate cancer by evaluation of levels of prostate cancer antigen (PCA), previous PCA levels are frequently used as a baseline for evaluating whether the individual patient experiences a change.

In another embodiment, the previous sample from the patient resulted in a positive diagnosis (i.e., tumor growth was positively identified). In this embodiment, the baseline provided by the previous sample is effectively a positive control for tumor growth, and the subsequent samplings of the patient are compared to this baseline to monitor the progress of the tumor growth and/or to evaluate the efficacy of a treatment which is being prescribed for the cancer. In this embodiment, it may also be beneficial to have a negative baseline level of biomarker expression or biological activity (i.e., a normal cell baseline control), so that a baseline for remission or regression of the tumor can be set. Monitoring of a patient's tumor growth can be used by the clinician to modify cancer treatment for the patient based on whether an increase or decrease in cell growth is indicated.

It will be clear to those of skill in the art that some samples to be evaluated will not readily provide an obvious autologous control sample, or it may be determined that collection of autologous control samples is too invasive and/or causes undue discomfort to the patient. In these instances, an alternate method of establishing a baseline level of biomarker expression or biological activity can be used, examples of which are described below.

Another method for establishing a baseline level of biomarker expression or biological activity is to establish a baseline level of biomarker expression or biological activity from control samples, and preferably control samples that were obtained from a population of matched individuals. It is preferred that the control samples are of the same sample type as the sample type to be evaluated for biomarker expression or biological activity (e.g., the same cell type, and preferably from the same tissue or organ).

According to the present invention, the phrase “matched individuals” refers to a matching of the control individuals on the basis of one or more characteristics which are suitable for the type of cell or tumor growth to be evaluated. For example, control individuals can be matched with the patient to be evaluated on the basis of gender, age, race, or any relevant biological or sociological factor that may affect the baseline of the control individuals and the patient (e.g., preexisting conditions, consumption of particular substances, levels of other biological or physiological factors). To establish a control or baseline level of biomarker expression or biological activity, samples from a number of matched individuals are obtained and evaluated for biomarker expression or biological activity. The sample type is preferably of the same sample type and obtained from the same organ, tissue or bodily fluid as the sample type to be evaluated in the test patient. The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values.

A baseline such as that described above can be a negative control baseline, such as a baseline established from a population of apparently normal control individuals. Alternatively, as discussed above, such a baseline can be established from a population of individuals that have been positively diagnosed as having cancer, and particularly, cancer of a specified stage, as set forth by the medical community, so that one or more baseline levels can be established for use in staging a cancer in the patient to be evaluated. Therefore, in one embodiment, the baseline level is one or more tumor control samples that are correlated with a particular stage of tumor development for that type of tumor. For example, tumor samples from an appropriate number of individuals that have been diagnosed as having a particular stage of a given cancer (e.g., Stage I colon cancer) are tested for biomarker expression or biological activity. The values obtained from these control samples are statistically processed to establish a suitable baseline level using methods standard in the art for establishing such values, and the baseline is noted as being indicative of that particular stage of cancer. Preferably, a similar value is determined for each of the established stages of the given cancer, so that a panel of baseline values, each representing a different stage of the cancer, is formed. The level of biomarker expression or biological activity in the patient sample is then compared to each of the baseline levels to determine to which baseline the biomarker level of the patient is statistically closest. It will be appreciated that a given patient sample may fall between baseline levels of two different stages such that the best diagnosis is that the patient tumor is at least at the lower stage, but is perhaps in the process of advancing to the higher stage. The data provided by this method can be used in conjunction with current cancer staging methods to assist the physician in the evaluation of the patient and in prescribing suitable treatment for the cancer.

It will be appreciated by those of skill in the art that a baseline need not be established for each assay as the assay is performed but rather, a baseline can be established by referring to a form of stored information regarding a previously determined baseline level of biomarker expression for a given control sample, such as a baseline level established by any of the above-described methods. Such a form of stored information can include, for example, but is not limited to, a reference chart, listing or electronic file of population or individual data regarding “normal” (negative control) or tumor positive (including staged tumors) biomarker expression; a medical chart for the patient recording data from previous evaluations; or any other source of data regarding baseline biomarker expression that is useful for the patient to be diagnosed.

After the level of biomarker expression or biological activity is detected in the sample to be evaluated for tumor cell growth, such level is compared to the established baseline level of biomarker expression or biological activity, determined as described above. Also, as mentioned above, preferably, the method of detecting used for the sample to be evaluated is the same or qualitatively and/or quantitatively equivalent to the method of detecting used to establish the baseline level, such that the levels of the test sample and the baseline can be directly compared. In comparing the test sample to the baseline control, it is determined whether the test sample has a measurable decrease or increase in biomarker expression or biological activity over the baseline level, or whether there is no statistically significant difference between the test and baseline levels. After comparing the levels of biomarker expression or biological activity in the samples, the final step of making a diagnosis, monitoring, or staging of the patient can be performed as discussed above.

According to the present invention, detection of an increased level of biomarker expression or biological activity in the sample to be evaluated (i.e., the test sample) as compared to the baseline level indicates that, as compared to the baseline sample, increased cell growth or tumorigenicity or a potential therefore is indicated in the cells corresponding to the test sample. This indication of increased tumorigenicity is evaluated based on what the baseline represents, and can mean: (1) a positive diagnosis of tumorigenicity (i.e., neoplastic transformation) or potential for tumor cell growth in the patient; (2) continued or increased tumorigenicity in a patient previously diagnosed with a cancer; and/or (3) a higher stage of tumorigenicity than that represented by the baseline. More specifically, if the baseline is a normal or negative control sample (i.e., autologous or otherwise established, such as from a population control), a detection of increased biomarker expression or biological activity in the test sample as compared to the control sample indicates that the cells in the test sample are undergoing (or are at risk of undergoing) increased, and likely inappropriate (i.e., tumorous, neoplastic) cell growth. If the baseline sample is a previous sample from the patient (or a population control) and is representative of a positive diagnosis of tumor cell growth in the patient (i.e., a positive control), a detection of increased biomarker expression or biological activity in the sample as compared to the baseline may indicate that the cells in the test sample are experiencing increased tumor growth or a potential therefore, which would suggest to a clinician that a treatment currently being prescribed, for example, is not controlling the tumor growth or that tumor growth in the patient has recurred. If the baseline sample is representative of a particular stage of tumor, a detection of increased biomarker expression or biological activity in the sample as compared to the baseline may indicate that the cells in the test sample are at a higher stage of tumor growth than the stage represented by the baseline sample.

Similarly, detection of a decreased level of biomarker expression or biological activity in the sample to be evaluated (i.e., the test sample) as compared to the baseline level indicates that, as compared to the baseline sample, decreased cell growth or tumorigenicity or a potential therefore is indicated in the test cells. This indication of decreased tumorigenicity is evaluated based on what the baseline represents, and can mean: (1) a negative diagnosis of tumorigenicity (neoplastic transformation) or potential for tumor cell growth in the patient; (2) reduced tumorigenicity in a patient previously diagnosed with a cancer; and/or (3) a lower stage of tumorigenicity than that represented by the baseline. More specifically, if the baseline is a normal or negative control (autologous or otherwise established, such as from a population control), a detection of decreased biomarker expression or biological activity in the test sample as compared to the control sample indicates that the cells in the test sample are also normal and are not predicted to be at risk of undergoing inappropriate (i.e., tumorous, neoplastic) cell growth. If the baseline sample is a previous sample from the patient (or from a population control) and is representative of a positive diagnosis of tumorigenicity in the patient (i.e., a positive control), a detection of decreased biomarker expression or biological activity in the sample as compared to the baseline indicates that the cells in the test sample are experiencing decreased tumorigenicity or a potential therefore, which suggests to a clinician, for a patient that has cancer, that a treatment currently being prescribed, for example, is successfully controlling the tumor growth or that a tumor in the patient is in remission or eliminated. If the baseline sample is representative of a particular stage of tumor, a detection of decreased biomarker expression or biological activity in the sample as compared to the baseline indicates that the cells in the test sample are at a lower stage of tumor growth than the stage represented by the baseline sample.

Finally, detection of biomarker expression that is not statistically significantly different than the biomarker expression or biological activity in the baseline sample indicates that, as compared to the baseline sample, no difference in tumorigenicity or a potential therefore is indicated in the test cells. This indication of effectively a “baseline level” of cell growth in the test cell is evaluated based on what the baseline represents, and can mean: (1) a negative or positive diagnosis of tumorigenicity (neoplastic transformation) or potential therefore in the patient; (2) unchanged tumorigenicity in a patient previously diagnosed with a cancer; and/or (3) a correlation with a stage of tumor growth that is represented by the baseline. More specifically, if the baseline is a normal or negative control (autologous or otherwise established, such as from a population control), detection of biomarker expression or biological activity in the test sample that is not statistically significantly different than the baseline sample indicates that the cells in the test sample are also normal and are not predicted to be at risk of undergoing inappropriate (i.e., tumorous, neoplastic) cell growth. If the baseline sample is a previous sample from the patient (or from a population control) and is representative of a positive diagnosis of tumor cell growth in the patient (i.e., a positive control), a detection of biomarker expression or biological activity in the sample that is not statistically significantly different than the baseline indicates that the cells in the test sample are experiencing tumor cell growth or a potential therefore, and the patient should be further evaluated for cancer. In a patient who has cancer and is being monitored for tumor progression, a detection of biomarker expression or biological activity in the test sample that is not statistically significantly different than the baseline sample indicates that the tumor is neither increasing (progressing) nor decreasing (regressing). Such a diagnosis might suggest to a clinician that a treatment currently being prescribed, for example, is ineffective in controlling the tumor growth or is preventing accelerated tumor growth, but is not causing tumor growth to regress. Finally, if the baseline sample is representative of a particular stage of tumor, a detection of biomarker expression or biological activity in the test sample that is not statistically significantly different than the baseline sample indicates that the cells in the test sample are at substantially the same stage of tumor growth as the stage represented by the baseline sample.

As discussed above, a positive diagnosis indicates that increased cell growth, and possibly tumor cell growth (neoplastic transformation), has occurred, is occurring, or is statistically likely to occur in the cells or tissue from which the sample was obtained. In order to establish a positive diagnosis, the level of biomarker activity is increased over the established baseline by an amount that is statistically significant (i.e., with at least a 95% confidence level, or p<0.05). Preferably, detection of at least about a 10% change in biomarker expression or biological activity in the sample as compared to the baseline level results in a positive diagnosis of increased cell growth for said sample, as compared to the baseline. More preferably, detection of at least about a 30% change in biomarker expression or biological activity in the sample as compared to the baseline level results in a positive diagnosis of increased cell growth for said sample, as compared to the baseline. More preferably, detection of at least about a 50% change, and more preferably at least about a 70% change, and more preferably at least about a 90% change, or any percentage change between 5% and higher in 1% increments (i.e., 5%, 6%, 7%, 8% . . . ) in biomarker expression or biological activity in the sample as compared to the baseline level results in a positive diagnosis of increased tumorigenicity for said sample. In one embodiment, a 1.5 fold change in biomarker expression or biological activity in the sample as compared to the baseline level results in a positive diagnosis of increased tumorigenicity for said sample. More preferably, detection of at least about a 3 fold change, and more preferably at least about a 6 fold change, and even more preferably, at least about a 12 fold change, and even more preferably, at least about a 24 fold change, or any fold change from 1.5 up in increments of 0.5 fold (i.e., 1.5, 2.0, 2.5, 3.0 . . . ) in biomarker expression or biological activity as compared to the baseline level, results in a positive diagnosis of increased tumorigenicity for said sample.

This method of diagnosis can be used specifically to determine the prognosis for cancer in the patient or to determine the susceptibility of the patient to a therapeutic treatment. In some embodiments, the method may be useful to monitor the progress of a patient undergoing therapeutic treatment for a tumor.

The present invention also includes a kit that utilizes the diagnostic methods of the present invention. The kit preferably contains any means of detecting the expression or activity of a biomarker of the present invention in a test sample, and preferably includes a probe, PCR primers, or an antibody, antigen binding peptide, or fragment thereof, that binds to a biomarker. The kit can include any reagent needed to perform a diagnostic method envisioned herein. The antibody, or fragment thereof, can be conjugated to another unit, for example a marker or immobilized to a solid carrier (substrate). The kit can also contain a second antibody for the detection of biomarker: antibody complexes. In one embodiment, the kit can contain a means for detecting a control marker characteristic of a cell type in the test sample. The antibody, or fragment thereof, may be present in free form or immobilized to a substrate such as a plastic dish, a test tube, a test rod and so on. The kit can also include suitable reagents for the detection of and/or for the labeling of positive or negative controls, wash solutions, dilution buffers and the like.

More specifically, according to the present invention, a means for detecting biomarker expression or biological activity can be any suitable reagent that can be used in a method for detection of biomarker expression or biological activity as described previously herein. Such reagents include, but are not limited to: a probe that hybridizes under stringent hybridization conditions to a nucleic acid molecule encoding the biomarker or a fragment thereof (including to a biomarker-specific regulatory region in the biomarker-encoding gene); RT-PCR primers for amplification of mRNA encoding the biomarker or a fragment thereof; and/or an antibody, antigen-binding fragment thereof or other antigen-binding peptide that selectively binds to the biomarker.

According to the present invention, a probe is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al., supra or Glick et al., supra).

Antibodies that selectively bind to a biomarker in the sample can be produced using information available in the art. Antibodies useful in the assay kit and methods of the present invention can include polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies is described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The invention also extends to non-antibody polypeptides, sometimes referred to as antigen binding partners or antigen binding peptides, which have been designed to bind selectively to the protein of interest (a biomarker). Examples of the design of such polypeptides, which possess a prescribed ligand specificity, are given in Beste et al. (Proc. Natl. Acad. Sci. 96:1898-1903, 1999), incorporated herein by reference in its entirety.

In one embodiment, a means for detecting a control marker that is characteristic of the cell type being sampled can generally be any type of reagent that can be used in a method of detecting the presence of a known marker in a sample, such as by a method for detecting the presence of a biomarker described previously herein. Specifically, the means is characterized in that it identifies a specific marker of the cell type being analyzed that positively identifies the cell type. For example, in a breast tumor assay, it is desirable to screen breast epithelial cells for the level of the biomarker expression and/or biological activity. Therefore, the means for detecting a control marker identifies a marker that is characteristic of an epithelial cell and preferably, a breast epithelial cell, so that the cell is distinguished from other cell types, such as a fibroblast. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

The means for detecting a biomarker and/or a control marker of the assay kit of the present invention can be conjugated to a detectable tag or detectable label. Such a tag can be any suitable tag which allows for detection of the reagents used to detect the biomarker or control marker and includes, but is not limited to, any composition or label detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵i, ³⁵S, ¹⁴C, or ³²p), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

In addition, the means for detecting of the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly effecting the activity and/or ability of the detection means to detect the desired target molecule. Exemplary organic solid supports include polymers such as polystyrene, nylon, phenol-formaldehyde resins, acrylic copolymers (e.g., polyacrylamide), stabilized intact whole cells, and stabilized crude whole cell/membrane homogenates. Exemplary biopolymer supports include cellulose, polydextrans (e.g., Sephadexe), agarose, collagen and chitin. Exemplary inorganic supports include glass beads (porous and nonporous), stainless steel, metal oxides (e.g., porous ceramics such as ZrO₂, TiO₂, Al₂O₃, and NiO) and sand.

According to the present invention, the method and assay for assessing the tumorigenicity of cells in a patient, as well as other methods disclosed herein, are suitable for use in a patient or cells from a patient or host that is a member of the Kingdom, Animalia, and particularly of the Vertebrate class, Mammalia, including, without limitation, primates, livestock and domestic pets (e.g., a companion animal). Most typically, a patient will be a human patient or host cells will be derived from human patients, although the use of the methods of the invention in any suitable non-human animal model or host cell is also encompassed.

All publications cited herein are incorporated by reference in their entirety.

The Examples, which follow, are illustrative of specific embodiments of the invention, and various uses thereof. They are set forth for explanatory purposes only, and are not to be taken as limiting the invention.

### EXAMPLES

### Example 1

The purpose of this experiment was to perform a nearly saturated genome wide GSE screen in a tumor cell line model for GSEs that protect cells against apoptosis.

1. V98 Vector Design and Construction

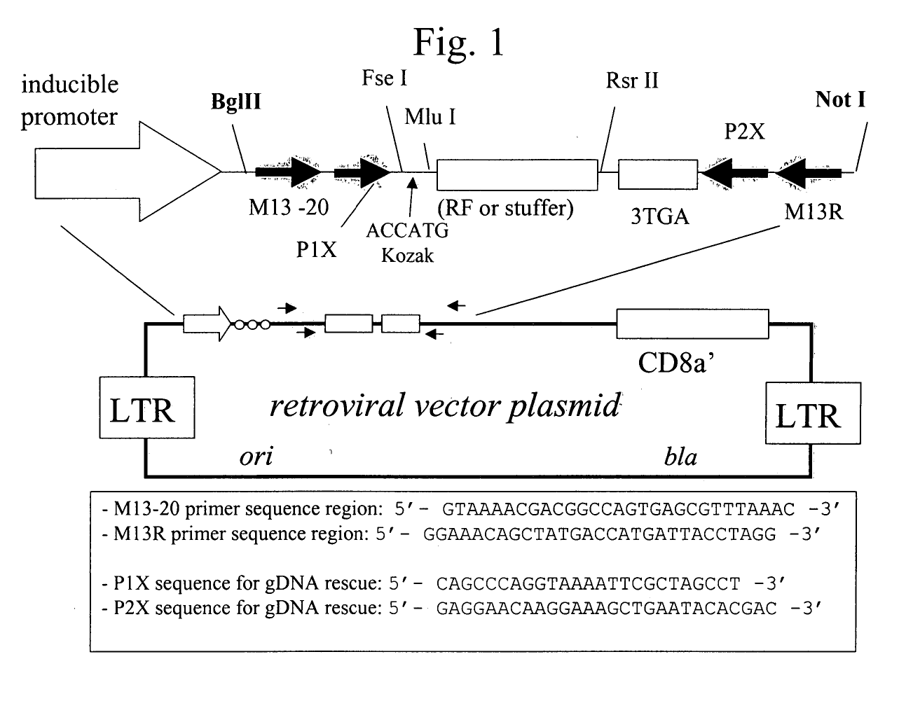

Vector V98 was created through modification of p610SL, a derivative of pLNCO₃ (B -D Chang and I. B. Roninson, Gene 183 (1996) 137-142.) A schematic of V98 is shown in FIG. 1. The region flanking the multiple cloning site (MCS) downstream of the inducible CMV promoter was re-engineered (1) to introduce restriction endonuclease sites for enzymes expected to occur with low frequency in the human genome [e.g., Fse I (1 per 170 kBp), Mlu I (1 per 300 kBp), and Rsr II (1 per 260 kBp)], (2) to introduce a short sequence of nucleic acid containing stop codons in all three DNA reading frames downstream of the MCS, (3) to introduce between the Fse I and Mlu I sites on the re-engineered vector backbone a Kozak sequence for efficient translation initiation of peptides encoded by random fragments cloned into the MCS (4) to introduce sequences complementary to well established DNA primers used for DNA sequencing (e.g., M13F-20 and M13R), to permit rapid and efficient sequencing of inserts cloned into the MCS, (5) to introduce sequences flanking the MCS, derived from the genome of Zea mays, and (6) to introduce into the MCS a “stuffer” fragment of about 2.2 kBp, which permits easy assessment of the completeness of vector digestion and selection of the appropriate backbone fragment during vector preparation.

A cDNA encoding the open reading frame of the murine Lyt-2-alpha′ gene was recovered from Marathon ready mouse spleen cDNA (Clontech) using PCR with the following conditions:5 μL of marathon ready cDNA, 5 μL 10× cDNA PCR buffer, 1 μL 10 mM dNTP mix, 1 μL Advantage 2 polymerase mix (Clontech, #8430-1), 1 μL of 10 μM upstream primer 5′- ACC ATG GCC TCA CCG TTG ACC CGC TTT -3′ (SEQ ID NO:81), 1 μL or 10 μM downstream primer 5′- CTA GCG GCT GTG GTA GCA GAT GAG A -3′ (SEQ ID NO:82), and 36 μL of water. Cycling parameters were:94° C. for 3 min.; followed by 30 cycles of 94° C. for 30 sec., 55° C. for 30 sec., and 72° C. for 2 minutes; followed by 72° C. for 10 minutes; followed by a 4° C. soak. The resultant PCR product of 669 nucleotides was subcloned into a pCRII TOPO vector (InVitrogen). Several independent clones were sequenced to confirm no mutations were introduced in the Lyt-2-alpha′ ORF by the PCR. One pCRII-TOPO-Lyt-2-alpha′ clone was shown to be free of mutations, clone #2. DNA from clone #2 was subjected to a second round of PCR (Vt=50 μL) using the following conditions: 1 ng plasmid DNA, 5 μL 10× cDNA PCR buffer, 0.8 μL of 10 mM dNTP mix, 1 μL of Advantage 2 polymerase mix (Clontech, #8430-1), 2.5 μL of 10 μM upstream primer 5′- CTA CGG ATC CAC CAT GGC CTC ACC GTT GA -3′ (SEQ ID NO:83) and 2.5 μL of 10 μM downstream primer 5′- GTA CAT CGA TCT AGC GGC TGT GGT AGC AGA TGA GA -3′ (SEQ ID NO:84). These primers permitted recovery the ORF of the Lyt-2-alpha′ gene flanked by BamHI (upstream) and ClaI restriction endonuclease sites. Cycling parameters were: 94° C. for 3 min.; followed by 30 cycles of 94° C. for 30 sec., 55° C. for 30 sec., and 72° C. for 2 minutes; followed by 72° C. for 10 minutes; followed by a 4° C. soak. The resulting 689-bp PCR product was purified from surrounding proteins and salts using a Qiagen PCR clean up kit following manufacturer's instructions. The purified Clone #2 DNA digested with Bam HI restriction endonuclease (NEB, #R0136S). The digested product was purified using a Qiagen PCR clean up kit and the buffer was changed. The digested DNA was then further digested with Cla I restriction endonuclease (NEB, #R0197S). The doubly restricted Clone #2 DNA was then subcloned into the backbone fragment of the 610SL retroviral vector produced by double digestion of 610SL with Bcl I (NEB, #R0160S) and Sfu I (Roche, #1243497) restriction endonucleases. Sequencing of DNA harvested from several independent bacterial colonies that were produced from this subcloning step yielded a clone that showed no mutations in the Lyt-2-alpha′ ORF. This clone was named V97.

The modifications to the MCS regions of vector 610SL were created by sequential cloning of various double stranded oligonucleotides containing the desired sequences into several precursor plasmids. Sequences designed to be located 5′ to the Fse I GSE cloning site in V98, e.g., M13F-20 primer site, primer site for P1X, were created by subcloning annealed oligonucleotides 5′- AGC TGT AAA ACG ACG GCC AGT GAG CGT TTA AAC GAA TTC CAG ACT AGT GGC CGG CCG TGC A -3′ (SEQ ID NO:85) and 5′- CGG CCG GCC ACT AGT CTG GAA TTC GTT TAA ACG CTC ACT GGC CGT CGT TTT AC -3′ (SEQ ID NO:86) into the vector pEFGP-1 (ClonTech) between the HinD III and Pst I sites, to create pEGFP5′. The duplex produced by annealing primers 5′- AAT TCT GCA GCC CAG GTA AAA TTC GCT AGC CT -3′ (SEQ ID NO:87) and 5′- CTA GAG GCT AGC GAA TTT TAC CTG GGC TGC AG -3′ (SEQ ID NO:88), which contains the priming site for P1X sequence, was subcloned between the Eco RI and Spe I sites of pEGFP5′ to yield pEGFP54. The modified 5′ region of the MCS was recovered from plasmid pEFGP54 as a Bgl II—Not I flanked fragment, and subcloned between the Bgl II and Not I sites of p610SL, to yield p610-E54P1. Sequences designed to be located 3′ to the Rsr II GSE cloning site in V98, e.g., 3 frame stop cassette, primer P2X, M13R sequencing primer, were created by subcloning of annealed oligonucleotides 5′- CGG TCC GTG AGT GAG TGA GGC GCG CC G GAT CCT AAC CTA GGT AAT CAT GGT CAT AGC TGT TTC CTG CAG GGC -3′ (SEQ ID NO:89) and 5′- GGC CGC CCT GCA GGA AAC AGC TAT GAC CAT GAT TAC CTA GGT TAG GAT CCG GCG CGC CTC ACT CAC TCA CGG ACC GTG CA -3′ (SEQ ID NO:90) into the vector pBlueScript II (Stratagene) between the Pst I and Not I sites, to create plasmid pBS3.3′. The duplex produced by annealing primers 5′- GAT CCC GGG TCG TGT ATT CAG CTT TCC TTG TTC CT -3′ (SEQ ID NO:91) and 5′- CTA GAG GAA CAA GGA AAG CTG AAT ACA CGA CCC GG -3′ (SEQ ID NO:92), which contains the priming site for P2× sequence, was subcloned between the BamH I and Avr II sites of pBS3.3′ to yield pBS3.3P12.

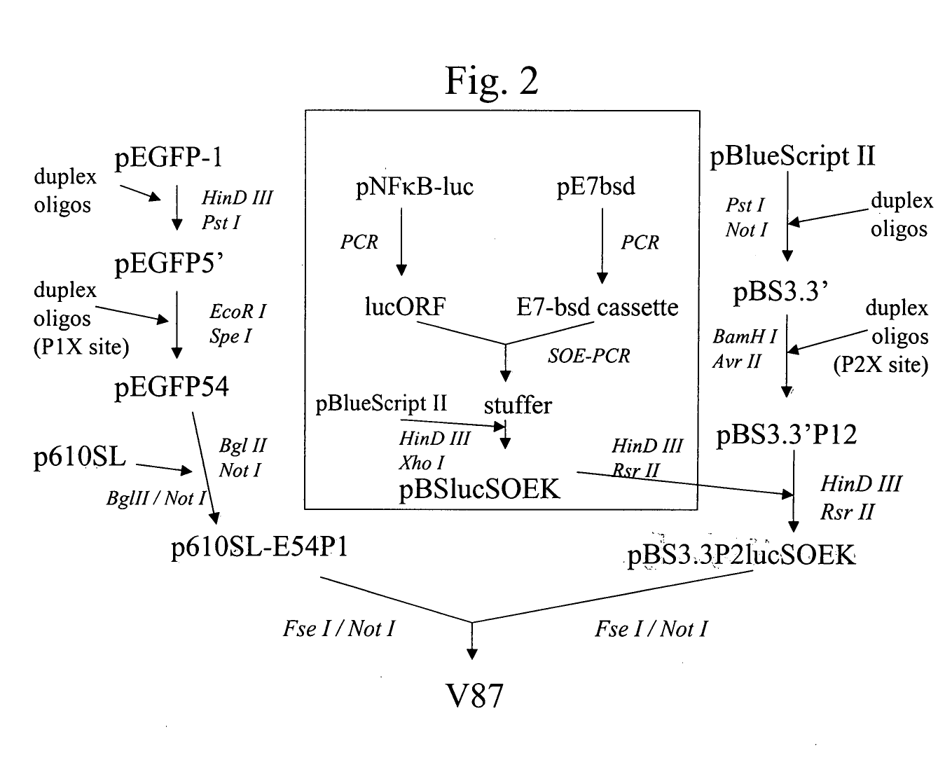

The stuffer fragment for V98 was designed to contain a luciferase ORF joined to a prokaryotic blasticidin S transferase (bsd) expression cassette, in order to yield a 2.2 kBp DNA fragment. The luciferase ORF and was created by PCR using the following primers 5′- CAT CAA GCT TGG CCG GCC ACC ATG GAC GCG TCC GAA GAC GCC AAA AAC ATA AAG -3′ (SEQ ID NO:93) and 5′- CAC GTG GAT ATC TTA CAA TTT GGA CTT TCC GCC CT -3′ (SEQ ID NO:94) to amplify the luciferase ORF from the plasmid pNFκB-luc (Strategene, #219078), while the bsd expression cassette was created by PCR using the primers 5′- TTG TAA GAT ATC CAC GTG TTG ACA ATT AAT C -3′ (SEQ ID NO:95) and 5′- CAT CAG ATC TGT CGA CCG GAC CGA CGC GTC CAC GAA GTG CTT AGC -3′ (SEQ ID NO:96) to amplify the E7-blasticindin S transferase open reading frame cassette from plasmid EM7-bsd. (InVitrogen, #V511-20). Both reactions were performed using the following cycling parameters: 95° C. for 3 min; followed by 30 cycles of 94° C. for 30 sec., 60° C. for 30 sec., 72° C. for 2 min.; followed by 72° C. for 10 min.; followed by a soak at 4° C. PCR products of the desired size were purified by agarose gel electrophoresis followed by recovery of the DNA from the gel using the Qiagen Gel Extraction kit according to manufacturer's instructions. The luciferase ORF and the bsd expression cassette were spliced together to generate a 2.2 kBp stuffer fragment using splice overlap extension PCR (Horton, R. M., Hunt, H. D., Ho, S. N., Pullen, J. K. and Pease, L. R. (1989)). Engineering hybrid genes without the use of restriction enzymes: gene splicing by overlap extension. Gene 77, 61-68) and the primers 5′- CAT CAA GCT TGG CCG GCC ACC ATG GAC GCG TCC GAA GAC GCC AAA AAC ATA AAG -3′ (SEQ ID NO:97) and 5′- CAT CAG ATC TGT CGA CCG GAC CGA CGC GTC CAC GAA GTG CTT AGC -3′ (SEQ ID NO:98). The resultant SOE PCR product was purified away from the proteins, primers, and salts using a Qiagen PCR clean up kit and following manufacturer's instructions. The product was digested with HinD III and Sal I restriction endonucleases, the restricted product was purified by agarose gel electrophoresis followed by recovery of the DNA from the gel using the Qiagen Gel Extraction kit according to manufacturer's instructions, and the purified DNA was subcloned between the HinD III (NEB, #R0104S) and Xho I (NEB, #R0146S) sites of plasmid pBluescript to yield pBSlucSOEK. Plasmid pBSlucSOEK was sequenced to confirm it was free of unwanted mutations, and the stuffer fragment recovered from the pBSlucSOEK as a HinD III and Rsr II fragment, which was purified by agarose gel electrophoresis, followed by recovery of the DNA from the gel using the Qiagen Gel Extraction kit according to manufacturer's instructions and subcloning of the fragment into the HinD III and Rsr II sites of plasmid pBS3.3′P12 to yield plasmid pBS33P21ucSOEK. Plasmid V87 was then constructed by recovering from plasmid pBS33P21ucSOEK the luciferase-E7-bsd stuffer fragment along with the 3′ flanking sequences as an Fse I—Not I flanked 2.2 kBp DNA product, which was purified by agarose gel electrophoresis followed by recovery of the DNA from the gel using the Qiagen Gel Extraction kit according to manufacturer's instructions. This 2.2 kBp DNA was subcloned between the Fse I and Not I site of plasmid p610-E54P1, to yield vector V87. A schematic drawing of the construction of V87 is shown in FIG. 2.

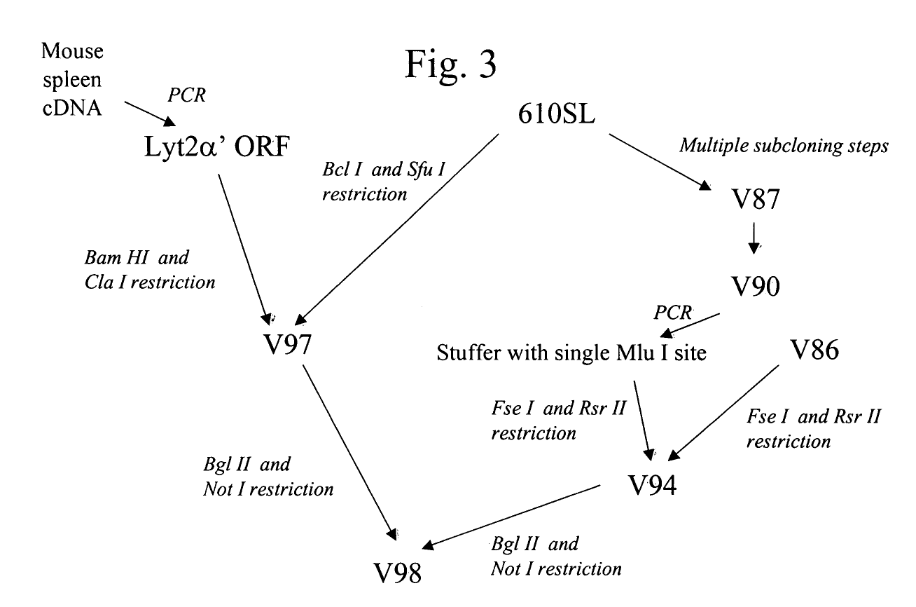

The downstream Mlu I site was removed from V87 by PCR amplification of the stuffer fragment of V90,a derivative of V87 containing the same stuffer as V87, using 1 ng of V90 template DNA and 2.5 μL of primer 5′- CAT CAA GCT TGG CCG GCC ACG CGT GTT GGT AAA ATG GAA GAC G -3′ (SEQ ID NO:99) and 2.5 μL of primer 5′- CAT GAG ATC TGT CGA CCG GAC CGC CAC GAA GTG CTT AAG C -3′ (SEQ ID NO:100) in a standard 50 μL PCR reaction using Taq DNA polymerase (Roche, 1146165). Cycling parameters were:94° C. for 3 min.; followed by 30 cycles of 94° C. for 20 sec., 55° C. for 20 sec., and 72° C. for 3 minutes; followed by 72° C. for 10 minutes; followed by a 4° C. soak. The resultant 2.2 kBp PCR product was purified from the proteins and salt using a Qiagen PCR clean up kit following manufacturer's instructions. The PCR product was digested with Fse I and Rsr II endonucleases, purified by agarose gel electrophoresis, and recovered from the gel using a Qiagen Gel Extraction kit according to manufacturer's instructions. The restricted and purified fragment was then subcloned into the Fse I and Rsr II sites of V86, a vector related to V87 but containing a stuffer fragment containing the luciferase ORF but not the bsd ORF, to yield V94. The MCS GSE cassette was recovered from V94 as a 2.2 kBp DNA fragment by digestion of V94 with Bgl II (NEB, #R0144S) and Not I (NEB, #R0189S) restriction endonucleases. Vector V98 was created by subcloning this 2.2 kBp DNA fragment from V94 into the Bgl II and Not I sites on the V97 backbone. A schematic of the construction of V98 is shown in FIG. 3.

2. Random Fragment Library Construction

For construction of the starting AOLC1U library, V98 vector described above was restricted at 37° C. for 3 hours, using Mlu I (NEB, #R0198S) and Rsr II restriction endonucleases. For construction of all other selected libraries, e.g., AOLC1A, AOLC1B, AOLC1C, V98 vector DNA was restricted at 37° C. for 3 hours, using Fse I and Rsr II restriction endonucleases. The vector DNA was purified from the digest using a Qiagen PCR clean up spin column according to manufacturer's instructions, and the vector backbone DNA was purified by subjecting the eluate from the column to agarose gel electrophoresis to resolve the various DNA digestion products according to mass. A gel slice containing the 7.7 kBp backbone fragment was excised, and the DNA recovered from the agarose slice using the Qiagen Gel Extraction kit according to manufacturer's instructions. The concentration of DNA present in the vector preparations was determined by ethidium bromide staining in an 0.8% agarose gel following electrophoresis, by comparison to a DNA sample composed of various bands of known size and mass (High DNA Mass Ladder, Life Technologies, 10406-016). Vector preparations were quality controlled in series of test ligations as follows: vector alone control reaction, composed of x μL vector DNA (30 fmol), z μL water, 4 μL 5× ligase buffer, 1 μL T4 DNA ligase (BRL, 5 U/μL, #15224-041), where x+z=15 μL; and a vector+insert reaction, composed of x μL vector DNA (30 fmol), y μL insert DNA (90 fmol), z μL water, 4 μL 5× ligase buffer, 1 μL T4 DNA ligase, where x+y+z=15 μL. Ligation reactions were incubated at 16° C. for at least 16 hours. At the end of the incubation period, ligation products were precipitated under ethanol, the ethanol decanted and the precipitate washed three times with 70% EtOH, and the pellet dried and resuspended in 20 μL of water. One microliter of resuspended DNA solution was electrotransformed into DH10B electrocompetent cells (Life Technologies, 18290-015) according to manufacturers instructions. Following transformation, bacteria was recovered in 960 μL of room temperature SOC media, and recovery mixtures incubated at 37° C. in a rotary shaker, 250-300 rpm, for at least 40 minutes. After the recovery period, 4 ten-fold serial dilutions of each transformation culture were created, i.e., 1:10, 1:100, 1:1000, and 1:10000, and 50 μL of each bacterial dilution mixture was plated on LB-agar plates containing carbenicillin. Plates were incubated at 37° C. overnight, and scored the following morning. Stock solutions of the double-restricted vector were aliquoted and stored frozen at −20° C., preferably in 30 fmol/tube amounts.

3. Preparation of Randomly Fragmented cDNAs from Cell Line mRNA

Total RNA was harvested from five colon cancer cell lines: HCT15, HT29, HCT116, SW480 and SW620, using a Qiagen RNeasy kit, according to manufacturer's instructions. Poly A+ mRNA was purified from the total RNA using an Oligotex kit (Qiagen) following manufacturer's instructions. The purified mRNA pools were fragmented by boiling the sample at 100° C. for 8 minutes, a time empirically determined to give a good distribution of cDNA fragments as demonstrated using a published fragmentation protocol (Gudkov and Roninson, “Isolation of Genetic Suppressor Elements (GSEs) from Random Fragment cDNA libraries in Retroviral Vectors,” Chapter 18, in Methods in Molecular Biology, Vol. 69:cDNA Library Protocols, p. 228, I. G. Cowell and C. A. Austin, eds. Humana Press Inc., Totowa N.J., 1997). Two parallel first strand cDNA synthesis reactions were performed using the fragmented mRNAs as template, with either an Asc I-N₉ random primer 5′- GTA ATA CGA CTC ACT ATA GGC GCG CCN₉-3′ (SEQ ID NO:101) or an Rsr II-N9 random primer 5′- GTA ATA CGA CTC ACT ATA GGC GGA CCG Ng -3′ (SEQ ID NO:102) and the SuperScript Choice Systems for cDNA synthesis (Gibco BRL) following manufacturer's instructions. Second strand synthesis was performed using the method of Gubler and Hoffmian Gene 25:263-9, 1989) again using the SuperScript kit. The resultant double strand cDNAs were blunted using T4 DNA polymerase (NEB, #M0203S), then ligated to double stranded adapters, produced by annealing the oligonucleotides 5′- ATG ATT ACG CCA CGG ACC GTC -3′ (SEQ ID NO:103) and 5′- GAC GGT CCG TGG CGT AAT CAT GGT CAT AGC -3′ (SEQ ID NO:104) to yield adapters containing an Rsr II restriction site, or the oligonucleotides 5′- ATG ATT ACG CCA GGC GCG CCA C -3′ (SEQ ID NO:105) and 5′- GTG GCG CGC CTG GCG TAA TCA TGG TCA TAG C -3′ (SEQ ID NO:106) to yield adapters containing an Asc I restriction site. cDNA samples prepared using the Asc-Ng primer were ligated to the adapters containing the Rsr II restriction site, while cDNA samples prepared using the Rsr II-Ng primer were ligated to adapters containing the Asc I restriction site. After ligation of the adapters to the cDNA fragments, excess adapters were removed by spun column chromatography.

4. Preparation of Normalized Inserts for Starting AOLC1U Library

Eluted cDNAs ligated to appropriate adapters were subjected to 22 cycles of PCR to amplify the inserts and to generate large quantities of insert for self-normalization: those inserts ligated to Rsr II adapters were amplified using the primers 5′- GCT ATG ACC ATG ATT ACG CCA CGG ACC GTC -3′ (SEQ ID NO:107) and 5′- GTA ATA CGA CTC ACT ATA GGC -3′ (SEQ ID NO:108), while inserts ligated to the Asc I adapters were amplified using the primers 5′- GCT ATG ACC ATG ATT ACG CCA GGC GCG CCA C -3′ (SEQ ID NO:109) and 5′- GTA ATA CGA CTC ACT ATA GGC GGA C -3′ (SEQ ID NO:110). PCR products were pooled, purified using a Qiagen PCR kit following manufacturer's instructions, evaporated to dryness using a rotary evaporator, and then resuspended in 25 μL of 10 mM Tris-HCl (pH 8.5). The cDNA fragments were normalized by self-hybridization and batch binding hydroxyapatite (HAP) chromatography, essentially as described by Gudkov and Roninson (op.cit)., except that samples were collected at 24, 48, 72, 96 hours. The extent of normalization was evaluated using real-time PCR at five loci: ACTB, TP53, CASP3, 18S and a mitochondrial locus.

Purified, normalized ssDNA fractions from the HAP columns were reconverted to dsDNA and amplified using PCR: again, those inserts ligated to Rsr II adapters were amplified using the primers 5′- GCT ATG ACC ATG ATT ACG CCA CGG ACC GTC -3′ (SEQ ID NO:111) and 5′- GTA ATA CGA CTC ACT ATA GGC -3′ (SEQ ID NO:112), while inserts ligated to the Asc I adapters were amplified using the primers 5′- GCT ATG ACC ATG ATT ACG CCA GGC GCG CCA C -3′ (SEQ ID NO:113) and 5′- GTA ATA CGA CTC ACT ATA GGC GGA C-3′ (SEQ ID NO:114). PCR products were purified using Qiagen PCR clean up columns following manufacturer's instructions, and the PCR products from the two types of inserts (e.g, those with Rsr II adapters and those with Asc adapters) were mixed one-to-one molar ratio. Approximately 100 ng of mixed PCR product was digested with Asc I (NEB, #R0558S) and Rsr II restriction endonucleases for 2 hours at 37° C. in multiple parallel reactions. DNA was recovered from the pooled digestions using a Qiagen PCR clean up kit following manufacturer's instructions. The concentration of restricted PCR products in the eluate was determined by resolving the DNA present in an aliquot of the eluate by 2% agarose gel electrophoresis. The fluorescent intensity of the PCR product band was compared to the intensity of bands in a DNA sample composed of a mixture of DNA fragments of known size and mass (Low DNA Mass Ladder, Life Technologies, 10068-013).

5. Isolation of GSEs for AOLC1A, AOLC1B, or AOLC1C Libraries.

Colon adenocarcinoma SW480 cells were engineered to stably express ecotropic retroviral receptor (EcoR), and the resulting cell line was termed SW480 E. Phoenix Eco retrovirus packaging cells were transfected with library plasmid DNA and SW480 E cells were transduced with viral supernatant harvested from the packaging cells. Floating SW480 E cells were collected at times 24, 48, 72 and 96 hours post-transduction and fixed with 100% methanol. Apoptotic cells were collected from all time points by staining the fixed cells with a monoclonal antibody against caspase-cleaved cytokeratin 18 (M30 CytoDeath Antibody, Roche Diagnostics), and selecting stained cells by fluorescence activated cell sorting (FACS). Genomic DNA was isolated from the collected cells (typically between 1×10⁵ and 2×10⁶ cells, depending upon the selection round) using the Qiagen DNeasy kit (Qiagen). Recovered genomic DNA was quantitated using the PicoGreen DNA quantitation kit (Molecular Probes) in a fluorometric assay performed according to manufacturer's instructions.

GSEs were recovered from the integrated proviruses contained in the harvested genomic DNA using PCR and the following reaction recipe:10 μL genomic DNA solution, about 1 μg DNA, 5 μL of 3.3 μM p5× primer 5′- TCT GCA GCC CAG GTA AAA TTC GCT AGC CTC TAG T -3′ (SEQ ID NO:115), 5 μL of 3.3 μM p6× primer 5′- GAG GAA CAA GGA AAG CTG AAT ACA CGA CCC GTG AT -3′ (SEQ ID NO:116), 2 μL of 10 mM dNTP mix, 17 μL of H₂O, 10 μL 5× PCR buffer, 1 μL of Thermozyme (InVitrogen E120-01). Cycling conditions for the PCR were: 95° C. for 3 min.; followed by 30 cycles of 95° C. for 30 sec., 68° C. for 30 sec., 72° C. for 1 min.; followed by 72° C. for 10 min., followed by a soak at 4° C. At least 10, and typically 96 reactions were performed in parallel.

Two hundred μL of pooled PCR product from the genomic PCR samples was purified from proteins and salts using a Qiagen PCR clean up kit following manufacturer's instructions. The concentration of PCR product in the eluate was determined by resolving the DNA present in an aliquot of the eluate by 2% agarose gel electrophoresis and then comparing the fluorescent intensity of the PCR product band to the intensity of bands in a DNA sample composed of a mixture of DNA fragments of known size and mass (Low DNA Mass Ladder, Life Technologies, 10068-013). Multiple parallel restriction digests were then set up using samples of the purified PCR product present in the eluate using the following recipe:10 μL 10× NEB buffer #4 (final 1× concentration:20 mM Tris-acetate, 10 mM magnesium acetate, 50 mM potassium acetate, 1 mM dithiothreitol), 1 μL 100× BSA (NEB), 7 μL Fse I restriction endonuclase (2 U/EL, NEB #R0588S), 3 μL Rsr II restriction endonuclease (4 U/EL, NEB #R0501S), X μL aliquot of PCR product, about 100 ng, 79 μL water, to bring total digestion volume up to 100 μL. Restriction digests were incubated at 37° C. for 3 hours. DNA products from the digest were separated from proteins and salts and concentrated using a Zymo DNA Clean & Concentrator-5 concentrator kit (#D4004), following the manufacturer's instructions with the following modifications: after addition of DNA binding buffer to each of the digestion reactions, all of the reactions were spun through the same column to concentrate 600 to 800 ng of digested insert onto a single column. Columns were washed according to the manufacturer's protocols, and the DNA eluted from the column by two sequential additions of 8 μL of 50 mM Tris-HCl, pH 8.5. DNA of desired sizes (100-500 bp) was recovered from the concentrated eluate by purification using gel electrophoresis on 1% low melting point agarose (NuSieve GTG agarose, FMC bioproducts) gels. DNA bands in the gel were visualized following ethidium bromide staining of the gel, using a hand-held shortwave ultraviolet light source. Gel slices containing the desired DNA were excised using clean razor blades, and DNA extracted from the gel slice using the Qiagen gel purification kit, following manufacturer's instructions. The concentration of restricted and purified PCR product was determined by ethidium bromide staining of an agarose gel containing an aliquot of the purified PCR product, and a DNA sample of known composition and mass, as described above.

5. cDNA Library Preparation

Ligation reactions for each batch of insert prepared were set up as follows: (reaction 1) Vector control reaction: x μL vector DNA (150 ng), z μL water, 4 μL 5× ligase buffer, 1 μL T4 DNA ligase (BRL, 5 U/μL, #15224-041), where x+z=15 μL; (reaction 2) Vector+insert: x μL vector DNA (150 ng), y μL insert DNA (12 ng); z μL water; 4 μL 5× ligase buffer; 1 μL T4 DNA ligase, where x+y+z=15 μL. Ligation mixtures were incubated at 4° C. for at least 16 hours. At the end of the ligation period, ligation products were precipitated under ethanol, the ethanol decanted and the precipitate washed three times with 70% EtOH, and the pellet dried and resuspended in 20 μL of water. One μL of resuspended ligation product was used to electrotransform DH10B electrocompetent cells (Life Technologies, 18290-015) according to manufacturers instructions; the balance of the ligation mixture was stored at −20° C. Following transformation, bacteria was recovered in 960 μL of room temperature SOC media, and recovery mixtures incubated at 37° C. in a rotary shaker, 250-300 rpm, for at least 40 minutes. After the recovery period, 4 ten-fold serially diluted samples (i.e., 1:10, 1:100, 1:1000, and 1:10000) of each transformation culture were set up, and 50 μL from each dilution was plated on LB-agar plates containing carbenicillin. Plates were incubated at 37° C. overnight, and colony counts for each plate scored the following morning.

Insert sizes in a subset of clones were determined by performing PCR directly on bacterial colonies as follows. A disposable pipette tip was used to harvest a single bacterial colony from the LB plate of interest. The colony was transferred into 25 μL of water, carefully swishing the tip to dislodge the bacterial colony. Five microliters of bacterial solution was spotted to an LB plate and allowed to incubate overnight. PCR was performed on the bacterial solution using the following recipe:2 μL 10 mM primer M13F(17) 5′-GTA AAA CGA CGG CCA GT-3′ (SEQ ID NO:117), 2 μL 10 mM primer p6× 5′- TCT GCA GCC CAG GTA AAA TTC GCT AGC CTC TAG T -3′ (SEQ ID NO:118), 4 μL 10× PCR buffer, 1 μL 25 mM dNTP mix, 0.5 μL Taq DNA polymerase (Roche, 1146165), 10.5 μL PCR grade water, 20 μL of bacterial solution. The cycling parameters were 95° C. for 3 min., then 25 cycles of 95° C. for 30 sec., 60° C. for 30 sec., 72° C. for 1 min., followed by 72° C. for 5 min., and a 4° C. soak. At the completion of the PCR, 10 μL of each PCR product was resolved on a 2% agarose gel containing ethidium bromide. DNA mobility for each of the samples was evaluated. The balance of the PCR product was submitted for DNA sequencing to determine the sequence content of the inserts for these clones.

Ligations described above were used for further electro-transformations. The calculated cfu/μg for each of the QC controlled ligations was used to compute the total number of electrotransformations required to achieve the required complexity for the library being constructed. Multiple electrotransformations were performed in parallel, using 1 μL of ligation mix per transformation as described above. At the end of the 40-minute recovery period following the electrotransformation, up to 10 independent transformations were pooled, and 50 μL from these pooled samples used to establish 4 ten-fold serially diluted samples (i.e., 1:10, 1:100, 1:1000, and 1:10000). Fifty μL of each serial dilution (i.e., 1:10, 1:100, 1:1000, and 1:10000) was plated on LB-agar plates containing carbenicillin. The remaining volumes of undiluted and diluted transformation solutions were used to seed a bacterial culture flask containing 0.5 L of LB broth, after which the seeded flask was incubated at 30° C. overnight, about 14-16 hours, in a rotary shaker at 300 rpm. Plates from the serially diluted samples were incubated at 37° C. overnight, and colony counts for each plate scored the following morning to determine the total number of colonies seeded into the 0.5 L culture. Library plasmid DNA was recovered from the 0.5 L cultures using a Qiagen Maxiprep plasmid kit, according to manufacturer's instructions.

The AOLC1U library was constructed using the normalized inserts prepared as described in section 4 above; the library was composed of greater than 80 million transformants. The AOLC1A library was constructed from GSEs recovered from apoptotic HCT116 cells collected 24, 48, 72 and 96 hours after transduction. The AOLC1B library was constructed from GSEs recovered from apoptotic HCT 116 cells collected 24, 48, and 72 hours after transduction. The AOLC1C library was constructed from GSEs recovered from apoptotic HCT116 cells collected 48 hours after transduction. It was found that the AOLC1C library was highly enriched for RPX and E. coli sequences; with the RPL5, RPL36, RPL8, Fau, RPL13a species being the majority species. To subtract these sequences from AOLC IC library, the following procedure was performed: (1) library DNA was linearized using FseI restriction endonuclease, (2) primers specific to selected RPX and E. coli species were annealed to linearized DNA and (3) DNA synthesis extended from the primer using Bst DNA polymerase, a polymerase that lacks 3′ exonuclease activity. Upon primer extension, the overhang of the FseI half-site adjacent to the insert will be lost, since extension products will yield blunt dsDNA. This blunted Fse I half-site will be incapable of adhering to the cohesive Fse I half-site present at the other end of the plasmid. Therefore, the DNA molecules to which primers have bound (e.g. the RPX and E. coli species) should have their Fse I sites blunted, and therefore be incapable of resealing by T4 DNA ligase. Hence, all linearized library DNA is treated with T4 DNA ligase, and the ligation products are transformed into electocompetent DH10B E.coli to generate a library enriched in sequences that do not contain RPX or E coli species. The enriched or subtracted library so created from the AOLC1C library was termed AOLC1CS. Sequencing of the AOLC1CS library showed that the targeted plasmids were substantially reduced in number, but they were still predominant species in the AOLC1CS library. Thus, the AOLC1CS library was subjected to another round of subtraction using the same method, with the resulting library termed AOLCICS2 (AOLC1C library after 2 rounds of subtraction). The primers used in this method to make library AOLC1CS were: RPS5: 5′- TCG TTC GAG GAG CCC TTG GCA GCA T-3′ (SEQ ID NO:119); RPL36A, 5′-CGC CCT TCC GCC ACG GCC GTC TCT -3′ (SEQ ID NO:120); RPL18 5′- GAA AGG ACC CGT CGC CAT GGG CCG T -3′ (SEQ ID NO:121); Fau, 5′- CAG TCG CCA ATA TGC AGC TCT TTG T -3′ (SEQ ID NO:122); RPL13A, 5′- CGA GGT ATG CTG CCC CAC AA -3′ (SEQ ID NO:123). For library AOLC1CS2, the above primers were used and these primers were added as well: RPS5, 5′- CGA GCG CCT GTG CAC AGC AGC CAG A -3′ (SEQ ID NO:124); RPL36A, 5′- GCG GGA CAT GAT TCG GGA GGT GTG T -3′ (SEQ ID NO:125); RPL8, 5′- CTG CGC GCC TGC GCG CCG TGG ATT T -3′ (SEQ ID NO:126); Fau, 5′- CTT CGA GGT GAC CGG CCA GGA AAC G -3′ (SEQ ID NO:127); RPL13A, 5′- CAG GCC GCT CTG GAC CGT CTC AAG G -3′ (SEQ ID NO:128); E coli, 5′-AAC GGT GGG CTT GTT GCT GCT CTG G -3′ (SEQ ID NO:129), 5′- ATT GGT ATT GGT AAC GGG CGT CAG G -3′ (SEQ ID NO:130), 5′- ACC ATC TTC CAG GCG CAG TTG AGT T -3′ (SEQ ID NO:131).

The target genes and encoded proteins identified by the present invention are explicitly disclosed in Table 1, which contains a common name for the gene and the GENBANK accession number, which can be retrieved from public sequence databases, as well as a sequence identifier for the nucleic acid sequence (first number) and encoded amino acid sequence (second number).

TABLE 1

| Accession | Common | Sequence Identifier | |
|---|---|---|---|
| Number | Name | (nucleic acid & protein) | Description |
| NM_001087 | AAMP | SEQ ID NO: 1 & 2 | angio-associated, migratory cell protein |
| NM_001109 | ADAM8 | SEQ ID NO: 3 & 4 | a disintegrin and metalloproteinase domain 8 |
| NM_139057 | ADAMTS17 | SEQ ID NO: 5 & 6 | a disintegrin-like and metalloprotease (reprolysin |
| | | | type) with thrombospondin type 1 motif, 17 |
| NM_004036 | ADCY3 | SEQ ID NO: 7 & 8 | adenylate cyclase 3 |
| NM_001619 | ADRBK1 | SEQ ID NO: 9 & 10 | adrenergic, beta, receptor kinase 1 |
| NM_006698 | BLCAP | SEQ ID NO: 11 & 12 | bladder cancer associated protein |
| NM_012264 | C22orf5 | SEQ ID NO: 13 & 14 | chromosome 22 open reading frame 5 |
| NM_004356 | CD81 | SEQ ID NO: 15 & 16 | CD81 antigen (target of antiproliferative antibody 1.) |
| NM_001769 | CD9 | SEQ ID NO: 17 & 18 | CD9 antigen (p24) |
| NM_001305 | CLDN4 | SEQ ID NO: 19 & 20 | claudin 4 |
| NM_001288 | CLIC1 | SEQ ID NO: 21 & 22 | chloride intracellular channel 1 |
| NM_058175 | COL6A2 | SEQ ID NO: 23 & 24 | collagen, type VI, alpha 2 |
| AF070636 or | CTL2 | SEQ ID NO: 25 & 26 | CTL2 gene |
| NM_020428 | | | |
| NM_001397 | ECE1 | SEQ ID NO: 27 & 28 | endothelin converting enzyme 1 |
| NM_004429 | EFNB1 | SEQ ID NO: 29 & 30 | ephrin-B1 |
| NM_004475 | FLOT2 | SEQ ID NO: 31 & 32 | flotillin 2 |
| AC011511 or | ICAM3 | SEQ ID NO: 33 & 34 | intercellular adhesion molecule 3 |
| BC058903 | | | |
| NM_006123 | IDS | SEQ ID NO: 35 & 36 | iduronate 2-sulfatase (Hunter syndrome) |
| NM_002226 | JAG2 | SEQ ID NO: 37 & 38 | jagged 2 |
| BC001699 | JAM1 | SEQ ID NO: 39 & 40 | junctional adhesion molecule 1 |
| NM_005567 | LGALS3BP | SEQ ID NO: 41 & 42 | lectin, galactoside-binding, soluble, 3 binding protein |
| XM_085426 | LOC146330 | SEQ ID NO: 43 & 44 | similar to possible G-protein receptor |
| BC020590 | LOC51107 | SEQ ID NO: 45 & 46 | CGI-78 protein |
| NM_000237 | LPL | SEQ ID NO: 47 & 48 | lipoprotein lipase |
| NM_002335 | LRP5 | SEQ ID NO: 49 & 50 | low density lipoprotein receptor-related protein 5 |
| NM_005581 | LU | SEQ ID NO: 51 & 52 | Lutheran blood group (Auberger b antigen included) |
| NM_005898 | M11S1 | SEQ ID NO: 53 & 54 | membrane component, chromosome 11, surface |
| | | | marker 1 |
| NM_007061 | MSE55 | SEQ ID NO: 55 & 56 | serum constituent protein |
| NM_006702 | NTE | SEQ ID NO: 57 & 58 | neuropathy target esterase |
| AK055605 or | PLXNA1 | SEQ ID NO: 59 & 60 | Homo sapiens cDNA FLJ31043 fis, clone |
| AK126101 | | | HSYRA2000248 (PLEXIN A1) or Homo sapiens |
| | | | cDNA FLJ44113 fis, clone TESTI4046487, highly |
| | | | similar to Mus musculus plexin A1 |
| AF034800 | PPF1A3 | SEQ ID NO: 61 & 62 | protein tyrosine phosphatase, receptor type, f |
| | | | polypeptide (PTPRF), interacting protein (liprin), |
| | | | alpha 3 |
| NM_145648 | PTR4 | SEQ ID NO: 63 & 64 | Homo sapiens peptide-histidine transporter 4 (PTR4), |
| | | | mRNA |
| NM_004207 | SLC16A3 | SEQ ID NO: 65 & 66 | solute carrier family 16 (monocarboxylic acid |
| | | | transporters), member 3 |
| NM_005628 | SLC1A5 | SEQ ID NO: 67 & 68 | solute carrier family 1 (neutral amino acid |
| | | | transporter), member 5 |
| NM_014437 | SLC39A1 | SEQ ID NO: 69 & 70 | solute carrier family 39 (zinc transporter), member 3 |
| NM_021102 | SPINT2 | SEQ ID NO: 71 & 72 | serine protease inhibitor, Kunitz type, 2 |
| NM_003714 | STC2 | SEQ ID NO: 73 & 74 | stanniocalcin 2 |
| NM_014452 | TNFRSF21 | SEQ ID NO: 75 & 76 | tumor necrosis factor receptor superfamily, member |
| | | | 21 |
| NM_003299 | TRA1 | SEQ ID NO: 77 & 78 | tumor rejection antigen (gp96) 1 |
| NM_017636 | TRPM4 | SEQ ID NO: 79 & 80 | transient receptor potential cation channel, subfamily |
| | | | M, member 4 |

It should be understood that the foregoing disclosure emphasizes certain specific embodiments of the invention and that all modifications or alternatives equivalent thereto are within the spirit and scope of the invention as set forth in the appended claims.

1. A method for identifying a compound for inducing apoptosis, comprising identifying an inhibitor of a target selected from the group consisting of: angio-associated, migratory cell protein (AAMP, comprising SEQ ID NO:2), a disintegrin and metalloproteinase domain 8 (ADAM8, comprising SEQ ID NO:4), a disintegrin-like and metalloprotease (reporlysin type) with thrombospondin type 1 motif, 17 (ADAMTS17, comprising SEQ ID NO:6), adenylate cyclase 3 (ADCY3, comprising SEQ ID NO:8), adrenergic beta receptor kinase 1 (ADRBK1, comprising SEQ ID NO:10), bladder cancer associated protein (BLCAP, comprising SEQ ID NO:12), chromosome 22 open reading frame 5 (C22orf5, comprising SEQ ID NO:14), CD81 antigen (target of antiproliferative antibody 1 (CD81, comprising SEQ ID NO:16), CD9 antigen (p24) (CD9, comprising SEQ ID NO:18), claudin 4 (CLDN4, comprising SEQ ID NO:20), chloride intracellular channel 1 (CLIC1, comprising SEQ ID NO:22), collagen, type VI, alpha 2 (COL6A2, comprising SEQ ID NO:24), CTL2 (CTL2, comprising SEQ ID NO:26), endothelin converting enzyme 1 (ECE1, comprising SEQ ID NO:28), ephrin-B 1 (EFNB 1, comprising SEQ ID NO:30), flotillin 2 (FLOT2, comprising SEQ ID NO:32), intercellular adhesion molecule 3 (ICAM3, comprising SEQ ID NO:34), iduronate 2-sulfatase (Hunter syndrome) (IDS, comprising SEQ ID NO:36), jagged 2 (JAG2, comprising SEQ ID NO:38), junctional adhesion molecule 1 (JAM 1, comprising SEQ ID NO:40), lectin, galactoside-binding soluble 3 binding protein (LGALS3BP, comprising SEQ ID NO:42), similar to possible G-protein receptor (LOC146330, comprising SEQ ID NO:44), CGI-78 protein (LOC51107, comprising SEQ ID NO:46), lipoprotein lipase (LPL, comprising SEQ ID NO:48), low density lipoprotein receptor-related protein 5 (LRP5, comprising SEQ ID NO:50), Lutheran blood group (Auberger b antigen included) (LU, comprising SEQ ID NO:52), membrane component, chromosome 11, surface marker 1 (M11S1, comprising SEQ ID NO:54), serum constituent protein (MSE55, comprising SEQ ID NO:56), neuropathy target esterase (NTE, comprising SEQ ID NO:58), Homo sapiens cDNA FL31043 fis, clone HSYRA2000248 (PLEXIN A1) or Homo sapiens cDNA FLJ44113 fis, clone TEST14046487, highly similar to Mus musculus plexin A1 (PLXNA1, comprising SEQ ID NO:60), protein tyrosine phosphatase, receptor type, f polypeptide (PTPRF), interacting protein )(liprin), alpha 3 (PPFIA3, comprising SEQ ID NO:62), Homo sapiens peptide-histidine transporter 4 (PTR4), mRNA (PTR4, comprising SEQ ID NO:64), solute carrier family 16 (moncarboxylic acid transporters) member 3 (SLC16A3, comprising SEQ ID NO:66), solute carrier family 1 (neutral amino acid transporter) member 5 (SLCIA5, comprising SEQ ID NO:68), solute carrier family 39 (zinc transporter) member 3 (SLC39A1, comprising SEQ ID NO:70), serine protease inhibitor, Kunitz type 2 (SPINT2, comprising SEQ ID NO:72), stanniocalcin 2 (STC2, comprising SEQ ID NO:74), tumor necrosis receptor superfamily member 21 (TNFRSF21, comprising SEQ ID NO:76), tumor rejection antigen (gp96) 1 (TRA1, comprising SEQ ID NO:78), and transient receptor potential cation channel, subfamily M member 4 (TRPM4, comprising SEQ ID NO:80).

2. The method of claim 1, further comprising assessing the ability of an identified inhibitor to induce apoptosis in a cell.

3. The method of claim 2, further comprising detecting whether a compound identified as inducing apoptosis inhibits growth of tumor cells.

4. The method of claim 1, wherein the step of identifying comprises identifying an inhibitor of expression or activity of the target.

5. The method of claim 1, comprising the steps of:
a) contacting a host cell with a putative regulatory compound, wherein the host cell expresses the target or a biologically active fragment thereof; and
b) detecting whether the putative regulatory compound inhibits the target or biologically active fragment thereof, wherein a putative regulatory compound that inhibits the target as compared to in the absence of the compound is indicated to be a candidate compound for the induction of apoptosis in a host cell.

6. The method of claim 5, wherein the host cell is a tumor cell line.

7. The method of claim 5, wherein the step of detecting is selected from the group consisting of:
a) detecting expression of the target in the presence of the putative regulatory compound; and
b) detecting activity of the target in the presence of the putative regulatory compound.

8. The method of claim 7, wherein the expression of the target is measured by polymerase chain reaction.

9. The method of claim 7, wherein the expression of the target is measured using an antibody or antigen binding partner that selectively binds to the target.

10. The method of claim 7, wherein the activity of the target is measured by measuring the amount of a product generated in a biochemical reaction mediated by the target.

11. The method of claim 7, wherein the activity of the target is measured by measuring the amount of a substrate consumed in a biochemical reaction mediated by the target.

12. The method of claim 1, comprising the steps of:
a) determining the three-dimensional structure of the target;
b) identifying the three-dimensional structure of a putative inhibitor by using computer software to model an interaction between the target structure and a structure of a test compound; and
c) synthesizing compounds identified in (b) and assaying the compounds in an in vitro assay to determine whether the compound inhibits the expression or activity of the target.

13. The method of claim 1, wherein the target has been validated as being involved in tumor cell growth.

14. The method of claim 14, wherein the target has been validated as being involved in tumor cell growth by a process comprising:
a) inhibiting the target in a cell by a method selected from the group consisting of gene knock-out, anti-sense oligonucleotide expression, use of RNAi molecules and GSE expression; and
b) assaying the cell for the ability of the cell to grow.

15. A method for inducing apoptosis, comprising inhibiting the expression or activity of a target or a gene encoding the target, wherein the target is selected from the group consisting of: angio-associated, migratory cell protein (AAMP, comprising SEQ ID NO:2), a disintegrin and metalloproteinase domain 8 (ADAM8, comprising SEQ ID NO:4), a disintegrin-like and metalloprotease (reporlysin type) with thrombospondin type 1 motif, 17 (ADAMTS17, comprising SEQ ID NO:6), adenylate cyclase 3 (ADCY3, comprising SEQ ID NO:8), adrenergic beta receptor kinase 1 (ADRBK1, comprising SEQ ID NO:10), bladder cancer associated protein (BLCAP, comprising SEQ ID NO:12), chromosome 22 open reading frame 5 (C22orf5, comprising SEQ ID NO:14), CD81 antigen (target of antiproliferative antibody 1 (CD81, comprising SEQ ID NO:16), CD9 antigen (p24) (CD9, comprising SEQ ID NO:18), claudin 4 (CLDN4, comprising SEQ ID NO:20), chloride intracellular channel 1 (CLIC1, comprising SEQ ID NO:22), collagen, type VI, alpha 2 (COL6A2, comprising SEQ ID NO:24), CTL2 (CTL2, comprising SEQ ID NO:26), endothelin converting enzyme 1 (ECE1, comprising SEQ ID NO:28), ephrin-B1 (EFNB1, comprising SEQ ID NO:30), flotillin 2 (FLOT2, comprising SEQ ID NO:32), intercellular adhesion molecule 3 (ICAM3, comprising SEQ ID NO:34), iduronate 2-sulfatase (Hunter syndrome) (IDS, comprising SEQ ID NO:36), jagged 2 (JAG2, comprising SEQ ID NO:38), junctional adhesion molecule 1 (JAM1, comprising SEQ ID NO:40), lectin, galactoside-binding soluble 3 binding protein (LGALS3BP, comprising SEQ ID NO:42), similar to possible G-protein receptor (LOC146330, comprising SEQ ID NO:44), CGI-78 protein (LOC51107, comprising SEQ ID NO:46), lipoprotein lipase (LPL, comprising SEQ ID NO:48), low density lipoprotein receptor-related protein 5 (LRP5, comprising SEQ ID NO:50), Lutheran blood group (Auberger b antigen included) (LU, comprising SEQ ID NO:52), membrane component, chromosome 11, surface marker 1 (M11S1, comprising SEQ ID NO:54), serum constituent protein (MSE55, comprising SEQ ID NO:56), neuropathy target esterase (NTE, comprising SEQ ID NO:58), Homo sapiens cDNA FL31043 fis, clone HSYRA2000248 (PLEXIN A1) or Homo sapiens cDNA FLJ44113 fis, clone TEST14046487, highly similar to Mus musculus plexin A1 (PLXNA1, comprising SEQ ID NO:60), protein tyrosine phosphatase, receptor type, f polypeptide (PTPRF), interacting protein )(liprin), alpha 3 (PPFIA3, comprising SEQ ID NO:62), Homo sapiens peptide-histidine transporter 4 (PTR4), mRNA (PTR4, comprising SEQ ID NO:64), solute carrier family 16 (moncarboxylic acid transporters) member 3 (SLC16A3, comprising SEQ ID NO:66), solute carrier family 1 (neutral amino acid transporter) member 5 (SLCIA5, comprising SEQ ID NO:68), solute carrier family 39 (zinc transporter) member 3 (SLC39A1, comprising SEQ ID NO:70), serine protease inhibitor, Kunitz type 2 (SPINT2, comprising SEQ ID NO:72), stanniocalcin 2 (STC2, comprising SEQ ID NO:74), tumor necrosis receptor superfamily member 21 (TNFRSF21, comprising SEQ ID NO:76), tumor rejection antigen (gp96) 1 (TRAI, comprising SEQ ID NO:78), and transient receptor potential cation channel, subfamily M member 4 (TRPM4, comprising SEQ ID NO:80).

16. The method of claim 15, wherein the step of inhibiting is conducted by contacting a cell with an inhibitor of the target, wherein the inhibitor induces apoptosis in the cell.

17. A method for the diagnosis of a tumor comprising:
a) detecting a level of expression or activity of at least one biomarker in a test sample from a patient to be diagnosed, wherein the biomarker is selected from the group consisting of: angio-associated, migratory cell protein (AAMP, comprising SEQ ID NO:2), a disintegrin and metalloproteinase domain 8 (ADAM8, comprising SEQ ID NO:4), a disintegrin-like and metalloprotease (reporlysin type) with thrombospondin type 1 motif, 17 (ADAMTS17, comprising SEQ ID NO:6), adenylate cyclase 3 (ADCY3, comprising SEQ ID NO:8), adrenergic beta receptor kinase 1 (ADRBK1, comprising SEQ ID NO:10), bladder cancer associated protein (BLCAP, comprising SEQ ID NO:12), chromosome 22 open reading frame 5 (C22orf5, comprising SEQ ID NO:14), CD81 antigen (target of antiproliferative antibody 1 (CD81, comprising SEQ ID NO:16), CD9 antigen (p24) (CD9, comprising SEQ ID NO:18), claudin 4 (CLDN4, comprising SEQ ID NO:20), chloride intracellular channel 1 (CLIC1, comprising SEQ ID NO:22), collagen, type VI, alpha 2 (COL6A2, comprising SEQ ID NO:24), CTL2 (CTL2, comprising SEQ ID NO:26), endothelin converting enzyme 1 (ECE1, comprising SEQ ID NO:28), ephrin-B1 (EFNB1, comprising SEQ ID NO:30), flotillin 2 (FLOT2, comprising SEQ ID NO:32), intercellular adhesion molecule 3 (ICAM3, comprising SEQ ID NO:34), iduronate 2-sulfatase (Hunter syndrome) (IDS, comprising SEQ ID NO:36), jagged 2 (JAG2, comprising SEQ ID NO:38), junctional adhesion molecule 1 (JAM1, comprising SEQ ID NO:40), lectin, galactoside-binding soluble 3 binding protein (LGALS3BP, comprising SEQ ID NO:42), similar to possible G-protein receptor (LOC146330, comprising SEQ ID NO:44), CGI-78 protein (LOC51107, comprising SEQ ID NO:46), lipoprotein lipase (LPL, comprising SEQ ID NO:48), low density lipoprotein receptor-related protein 5 (LRP5, comprising SEQ ID NO:50), Lutheran blood group (Auberger b antigen included) (LU, comprising SEQ ID NO:52), membrane component, chromosome 11, surface marker 1 (M11S1, comprising SEQ ID NO:54), serum constituent protein (MSE55, comprising SEQ ID NO:56), neuropathy target esterase (NTE, comprising SEQ ID NO:58), Homo sapiens cDNA FL31043 fis, clone HSYRA2000248 (PLEXIN A1) or Homo sapiens cDNA FLJ44113 fis, clone TEST14046487, highly similar to Mus musculus plexin A1 (PLXNA1, comprising SEQ ID NO:60), protein tyrosine phosphatase, receptor type, f polypeptide (PTPRF), interacting protein )(liprin), alpha 3 (PPFIA3, comprising SEQ ID NO:62), Homo sapiens peptide-histidine transporter 4 (PTR4), mRNA (PTR4, comprising SEQ ID NO:64), solute carrier family 16 (moncarboxylic acid transporters) member 3 (SLC16A3, comprising SEQ ID NO:66), solute carrier family 1 (neutral amino acid transporter) member 5 (SLCIA5, comprising SEQ ID NO:68), solute carrier family 39 (zinc transporter) member 3 (SLC39A1, comprising SEQ ID NO:70), serine protease inhibitor, Kunitz type 2 (SPINT2, comprising SEQ ID NO:72), stanniocalcin 2 (STC2, comprising SEQ ID NO:74), tumor necrosis receptor superfamily member 21 (TNFRSF21, comprising SEQ ID NO:76), tumor rejection antigen (gp96) 1 (TRA1, comprising SEQ ID NO:78), and transient receptor potential cation channel, subfamily M member 4 (TRPM4, comprising SEQ ID NO:80);
b) comparing the level of expression or activity of the biomarker in the test sample to a baseline level of biomarker expression or activity established from a control sample; wherein detection of a statistically significant difference in the expression or activity of the biomarker in the test sample, as compared to the baseline level of the expression or biological activity of the biomarker, is an indicator of a difference in the tumorigenicity or potential therefore of cells in the patient.

18. The method of claim 17, wherein the step of detecting comprises detecting biomarker mRNA transcription in the test sample.

19. The method of claim 18, wherein the step of detecting is by a method selected from the group consisting of polymerase chain reaction (PCR), reverse transcriptase-PCR (RT-PCR), in situ hybridization, Northern blot, sequence analysis, gene microarray analysis, and detection of a reporter gene.

20. The method of claim 17, wherein the step of detecting comprises detecting the biomarker protein in the test sample.

21. The method of claim 20, wherein the step of detecting is by a method selected from the group consisting of immunoblot, enzyme-linked immunosorbant assay (ELISA), radioimmunoassay (RIA), immunoprecipitation, immunohistochemistry and immunofluorescence.

22. The method of claim 17, wherein the step of detecting comprises detecting biomarker biological activity in the test sample.

23. The method of claim 17, wherein detection of a statistically significant difference in the level of biomarker expression or activity in the test sample as compared to the baseline level, with a confidence of p<0.05, indicates that the cells in the test sample have a difference in tumorigenicity or potential therefore as compared to the control sample.

24. The method of claim 17, wherein the test sample is from a patient being diagnosed for cancer and wherein the baseline level is established from a control sample that is established as non-tumorigenic.

25. The method of claim 24, wherein an increase in the level of biomarker expression or activity of the test sample as compared to the baseline level of expression or activity indicates that cells from which the test sample was derived are predicted to be tumorigenic or predisposed to becoming tumorigenic.

26. The method of claim 17, wherein the test sample is from a patient who is known to have cancer, and wherein the baseline level comprises a level of biomarker expression or activity from a previous tumor cell sample from the patient;
wherein a statistically significant decrease in the level of biomarker expression or activity in the test sample as compared to the first baseline level of expression or activity from the previous tumor cell sample, indicates that the test sample is less tumorigenic than the previous tumor cell sample;
and wherein a statistically significant increase in the level of biomarker expression or activity in the test sample as compared to the first baseline level of expression or activity, indicates that the test sample is more tumorigenic than the previous tumor cell sample.

27. The method of claim 26, wherein the method further comprises a step (c) of modifying cancer treatment for the patient based on whether an increase or decrease in tumorigenicity is indicated in step (b).

28. The method of claim 17, wherein the baseline level is established by a method selected from the group consisting of:
(1) establishing a baseline level of biomarker expression or activity in an autologous control sample from the patient, wherein the autologous sample is from a same cell type, tissue type or bodily fluid type as the test sample of step (a);
(2) establishing a baseline level of biomarker expression or activity from at least one previous detection of biomarker expression or activity in a previous test sample from the patient, wherein the previous test sample was of a same cell type, tissue type or bodily fluid type as the test sample of step (a); and
(3) establishing a baseline level of biomarker expression or activity from an average of control samples of a same cell type, tissue type or bodily fluid type as the test sample of step (a), the control samples having been obtained from a population of matched individuals.

29. The method of claim 17, wherein the patient test sample is immobilized on a substrate.

30. The method of claim 17, wherein the test sample is a bodily fluid sample.

31. The method of claim 17, wherein the biomarker level is determined by contacting the patient test sample with an antibody or a fragment thereof that selectively binds specifically to the biomarker, and determining whether the antibody or fragment thereof has bound to the marker.

32. The method of claim 17, wherein the method is used to determine the prognosis for cancer in the patient.

33. The method of claim 17, wherein the method is used to determine the susceptibility of the patient to a therapeutic treatment.


SEQUENCE LISTING (131 sequences)

SEQ ID NO: 1 - DNA, 1762 bp, Homo sapiens
  CDS (1)..(1359)
atggactctgggaggcgtttgggcccagagaagtggatccgccgcttgcgccgcatggagtccgaatcggaaagcggggctgctgctgacacccccccactggagaccctaagcttccatggtgatgaagagattatcgaggtggtagaacttgatcccggtccgccggacccagatgacctggcccaggagatggaagatgtggactttgaggaagaagaggaggaagagggcaacgaagagggctgggttctagaaccccaggaaggggtggtcggcagcatggagggccccgacgatagcgaggtcacctttgcattgcactcagcatctgtgttttgtgtgagcctggaccccaagaccaataccttggcagtgaccgggggtgaagatgacaaagccttcgtatggcggctcagcgatggggagctgctctttgagtgtgcaggccataaagactctgtgacttgtgctggtttcagccatgactccactctagtggccacaggggacatgagtggcctcttgaaagtgtggcaggtggacactaaggaggaggtctggtcctttgaagcgggagacctggagtggatggagtggcatcctcgggcacctgtcctgttggcgggcacagctgacggcaacacctggatgtggaaagtcccgaatggtgactgcaagaccttccagggtcccaactgcccagccacctgtggccgagtcctccctgatgggaagagagctgtggtaggctatgaagatgggaccatcaggatttgggacctgaagcagggaagccctatccatgtactgaaagggactgagggtcaccagggcccactcacctgtgttgctgccaaccaggatggcagcttgatcctaactggctctgtggactgccaggccaagctggtcagtgccaccaccggcaaggtggtgggtgtttttagacctgagactgtggcctcccagcccagcctgggagaaggggaggagagtgagtccaactcggtggagtccttgggcttctgcagtgtgatgcccctggcagctgttggctacctggatgggaccttggccatctatgacctggctacgcagactcttaggcatcagtgtcagcaccagtcgggcatcgtgcagctgctgtgggaggcaggcactgccgtggtatatacctgcagcctggatggcatcgtgcgcctctgggacgcccggaccggccgcctgcttactgactaccggggccacacggctgagatcctggactttgccctcagcaaagatgcctccctggtggtgaccacgtcaggagaccacaaagcgaaagtattttgtgtccaaaggcctgaccgttaatggctgcagcccctgcctgtgtgtctggtgttgaggggacgaagggacccctgcccctgtctgccagcagaggcagtagggcacagagggaagaggagggtggggccctggatgactttccagcctcttcaactgacttgctcccctctccttttcttctctttagagacccagcccagggccctcccacccttgcccagacctggtgggcccttcagagggaggggtggacctgtttctctttcactttcatttgctggtgtgagccatggggtgtgtatttgtatgtggggagtaggtgtttgaggttcccgttctttcccttcccaagtctctgggggtggaaaggaggaagagatactagttaaagattttaaaaatgtaaataaaatatacttcccag

SEQ ID NO: 2 - protein, 452 aa, Homo sapiens
MDSGRRLGPEKWIRRLRRMESESESGAAADTPPLETLSFHGDEEIIEVVELDPGPPDPDDLAQEMEDVDFEEEEEEEGNEEGWVLEPQEGVVGSMEGPDDSEVTFALHSASVFCVSLDPKTNTLAVTGGEDDKAFVWRLSDGELLFECAGHKDSVTCAGFSHDSTLVATGDMSGLLKVWQVDTKEEVWSFEAGDLEWMEWHPRAPVLLAGTADGNTWMWKVPNGDCKTFQGPNCPATCGRVLPDGKRAVVGYEDGTIRIWDLKQGSPIHVLKGTEGHQGPLTCVAANQDGSLILTGSVDCQAKLVSATTGKVVGVFRPETVASQPSLGEGEESESNSVESLGFCSVMPLAAVGYLDGTLAIYDLATQTLRHQCQHQSGIVQLLWEAGTAVVYTCSLDGIVRLWDARTGRLLTDYRGHTAEILDFALSKDASLVVTTSGDHKAKVFCVQRPDR

SEQ ID NO: 3 - DNA, 3236 bp, Homo sapiens
  CDS (10)..(2484)
gacccggccatgcgcggcctcgggctctggctgctgggcgcgatgatgctgcctgcgattgcccccagccggccctgggccctcatggagcagtatgaggtcgtgttgccgcggcgtctgccaggcccccgagtccgccgagctctgccctcccacttgggcctgcacccagagagggtgagctacgtccttggggccacagggcacaacttcaccctccacctgcggaagaacagggacctgctgggttccggctacacagagacctatacggctgccaatggctccgaggtgacggagcagcctcgcgggcaggaccactgcttataccagggccacgtagaggggtacccggactcagccgccagcctcagcacctgtgccggcctcaggggtttcttccaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggactctctgccatcccgagagacccgctacgtggagctgtatgtggtcgtggacaatgcagagttccagatgctggggagcgaagcagccgtgcgtcatcgggtgctggaggtggtgaatcacgtggacaagctatatcagaaactcaacttccgtgtggtcctggtgggcctggagatttggaatagtcaggacaggttccacgtcagccccgaccccagtgtcacactggagaacctcctgacctggcaggcacggcaacggacacggcggcacctgcatgacaacgtacagctcatcacgggtgtcgacttcaccgggactactgtggggtttgccagggtgtccgccatgtgctcccacagctcaggggctgtgaaccaggaccacagcaagaaccccgtgggcgtggcctgcaccatggcccatgagatgggccacaacctgggcatggaccatgatgagaacgtccagggctgccgctgccaggaacgcttcgaggccggccgctgcatcatggcaggcagcattggctccagtttccccaggatgttcagtgactgcagccaggcctacctggagagctttttggagcggccgcagtcggtgtgcctcgccaacgcccctgacctcagccacctggtgggcggccccgtgtgtgggaacctgtttgtggagcgtggggagcagtgcgactgcggcccccccgaggactgccggaaccgctgctgcaactctaccacctgccagctggctgagggggcccagtgtgcgcacggtacctgctgccaggagtgcaaggtgaagccggctggtgagctgtgccgtcccaagaaggacatgtgtgacctcgaggagttctgtgacggccggcaccctgagtgcccggaagacgccttccaggagaacggcacgccctgctccgggggctactgctacaacggggcctgtcccacactggcccagcagtgccaggccttctgggggccaggtgggcaggctgccgaggagtcctgcttctcctatgacatcctaccaggctgcaaggccagccggtacagggctgacatgtgtggcgttctgcagtgcaagggtgggcagcagcccctggggcgtgccatctgcatcgtggatgtgtgccacgcgctcaccacagaggatggcactgcgtatgaaccagtgcccgagggcacccggtgtggaccagagaaggtttgctggaaaggacgttgccaggacttacacgtttacagatccagcaactgctctgcccagtgccacaaccatggggtgtgcaaccacaagcaggagtgccactgccacgcgggctgggccccgccccactgcgcgaagctgctgactgaggtgcacgcagcgtccgggagcctccccgtcctcgtggtggtggttctggtgctcctggcagttgtgctggtcaccctggcaggcatcatcgtctaccgcaaagcccggagccgcatcctgagcaggaacgtggctcccaagaccacaatggggcgctccaaccccctgttccaccaggctgccagccgcgtgccggccaagggcggggctccagccccatccaggggcccccaagagctggtccccaccacccacccgggccagcccgcccgacacccggcctcctcggtggctctgaagaggccgccccctgctcctccggtcactgtgtccagcccacccttcccagttcctgtctacacccggcaggcaccaaagcaggtcatcaagccaacgttcgcacccccagtgcccccagtcaaacccggggctggtgcggccaaccctggtccagctgagggtgctgttggcccaaaggttgccctgaagccccccatccagaggaagcaaggagccggagctcccacagcaccctaggggggcacctgcgcctgtgtggaaatttggagaagttgcggcagagaagccatgcgttccagccttccacggtccagctagtgccgctcagccctagaccctgactttgcaggctcagctgctgttctaacctcagtaatgcatctacctgagaggctcctgctgtccacgccctcagccaattccttctccccgccttggccacgtgtagccccagctgtctgcaggcaccaggctgggatgagctgtgtgcttgcgggtgcgtgtgtgtgtacgtgtctccaggtggccgctggtctcccgctgtgttcaggaggccacatatacagcccctcccagccacacctgcccctgctctggggcctgctgagccggctgccctgggcacccggttccaggcagcacagacgtggggcatccccagaaagactccatcccaggaccaggttcccctccgtgctcttcgagagggtgtcagtgagcagactgcaccccaagctcccgactccaggtcccctgatcttgggcctgtttcccatgggattcaagagggacagccccagctttgtgtgtgtttaagcttaggaatgccctttatggaaagggctatgtgggagagtcagctatcttgtctggttttcttgagacctcagatgtgtgttcagcagggctgaaagcttttattctttaataatgagaaatgtatattttactaataaattattgaccgagttctgtagattcttgttaga

SEQ ID NO: 4 - protein, 824 aa, Homo sapiens
MRGLGLWLLGAMMLPAIAPSRPWALMEQYEVVLPRRLPGPRVRRALPSHLGLHPERVSYVLGATGHNFTLHLRKNRDLLGSGYTETYTAANGSEVTEQPRGQDHCLYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAASGSLPVLVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQVIKPTFAPPVPPVKPGAGAANPGPAEGAVGPKVALKPPIQRKQGAGAPTAP

SEQ ID NO: 5 - DNA, 3470 bp, Homo sapiens
  CDS (24)..(3311)
gggggctgccggtcccgggtaccatgtgtgacggcgccctgctgcctccgctcgtcctgcccgtgctgctgctgctggtttggggactggacccgggcacagctgtcggcgacgcggcggccgacgtggaggtggtgctcccgtggcgggtgcgccccgacgacgtgcacctgccgccgctgcccgcagcccccgggccccgacggcggcgacgcccccgcacgcccccagccgccccgcgcgcccggcccggagagcgcgccctgctgctgcacctgccggccttcgggcgcgacctgtaccttcagctgcgccgcgacctgcgcttcctgtcccgaggcttcgaggtggaggaggcgggcgcggcccggcgccgcggccgccccgccgagctgtgcttctactcgggccgtgtgctcggccaccccggctccctcgtctcgctcagcgcctgcggcgccgccggcggcctggttggcctcattcagcttgggcaggagcaggtgctaatccagcccctcaacaactcccagggcccattcagtggacgagaacatctgatcaggcgcaaatggtccttgacccccagcccttctgctgaggcccagagacctgagcagctctgcaaggttctaacagaaaagaagaagccgacgtggggcaggccttcgcgggactggcgggagcggaggaacgctatccggctcaccagcgagcacacggtggagaccctggtggtggccgacgccgacatggtgcagtaccacggggccgaggccgcccagaggttcatcctgaccgtcatgaacatggtatacaatatgtttcagcaccagagcctggggattaaaattaacattcaagtgaccaagcttgtcctgctacgacaacgtcccgctaagttgtccattgggcaccatggtgagcggtccctggagagcttctgtcactggcagaacgaggagtatggaggagcgcgatacctcggcaataaccaggttcccggcgggaaggacgacccgcccctggtggatgctgctgtgtttgtgaccaggacagatttctgtgtacacaaagatgaaccgtgtgacactgttggaattgcttacttaggaggtgtgtgcagtgctaagaggaagtgtgtgcttgccgaagacaatggtctcaatttggcctttaccatcgcccatgagctgggccacaacttgggcatgaaccacgacgatgaccactcatcttgcgctggcaggtcccacatcatgtcaggagagtgggtgaaaggccggaacccaagtgacctctcttggtcctcctgcagccgagatgaccttgaaaacttcctcaagtcaaaagtcagcacctgcttgctagtcacggaccccagaagccagcacacagtacgcctcccgcacaagctgccgggcatgcactacagtgccaacgagcagtgccagatcctgtttggcatgaatgccaccttctgcagaaacatggagcatctaatgtgtgctggactgtggtgcctggtagaaggagacacatcctgcaagaccaagctggaccctcccctggatggcaccgagtgtggggcagacaagtggtgccgcgcgggggagtgcgtgagcaagacgcccatcccggagcatgtggacggagactggagcccgtggggcgcctggagcatgtgcagccgaacatgtgggacgggagcccgcttcaggcagaggaaatgtgacaaccccccccctgggcctggaggcacacactgcccgggtgccagtgtagaacatgcggtctgcgagaacctgccctgccccaagggtctgcccagcttccgggaccagcagtgccaggcacacgaccggctgagccccaagaagaaaggcctgctgacagccgtggtggttgacgataagccatgtgaactctactgctcgcccctcgggaaggagtccccactgctggtggccgacagggtcctggacggtacaccctgcgggccctacgagactgatctctgcgtgcacggcaagtgccagaaaatcggctgtgacggcatcatcgggtctgcagccaaagaggacagatgcggggtctgcagcggggacggcaagacctgccacttggtgaagggcgacttcagccacgcccgggggacagctctcaaagactcgggtaaggggtccatcaacagtgactggaagatagagctccccggagagttccagattgcaggcacaactgttcgctatgtgagaagggggctgtgggagaagatctctgccaagggaccaaccaaactaccgctgcacttgatggtgttgttatttcacgaccaagattatggaattcattatgaatacactgttcctgtaaaccgcactgcggaaaatcaaagcgaaccagaaaaaccgcaggactctttgttcatctggacccacagcggctgggaagggtgcagtgtgcagtgcggcggaggggagcgcagaaccatcgtctcgtgtacacggattgtcaacaagaccacaactctggtgaacgacagtgactgccctcaagcaagccgcccagagccccaggtccgaaggtgcaacttgcacccctgccagtcacggtgggtggcaggcccgtggagcccctgctcggcgacctgtgagaaaggcttccagcaccgggaggtgacctgcgtgtaccagctgcagaacggcacacacgtcgctacgcggcccctctactgcccgggcccccggccggcggcagtgcagagctgtgaaggccaggactgcctgtccatctgggaggcgtctgagtggtcacagtgctctgccagctgtggtaaaggggtgtggaaacggaccgtggcgtgcaccaactcacaagggaaatgcgacgcatccacgaggccgagagccgaggaggcctgcgaggactactcaggctgctacgagtggaaaactggggactggtctacgtgctcgtcgacctgcgggaagggcctgcagtcccgggtggtgcagtgcatgcacaaggtcacagggcgccacggcagcgagtgccccgccctctcgaagcctgccccctacagacagtgctaccaggaggtctgcaacgacaggatcaacgccaacaccatcacctccccccgccttgctgctctgacctacaaatgcacacgagaccagtggacggtatattgccgggtcatccgagaaaagaacctctgccaggacatgcggtggtaccagcgctgctgccagacctgcagggacttctatgcaaacaagatgcgccagccaccgccgagctcgtgacacgcagtcccaagggtcgctcaaagctcagactcaggtctgaaacccacccacccgcaagcctaccagccttgtggccacgcccccacccggctgccacaagaatccaactgcatagaacatgagcgtggacttggaaaaaaaaaaaaaaaaaaaaaa

SEQ ID NO: 6 - protein, 1095 aa, Homo sapiens
MCDGALLPPLVLPVLLLLVWGLDPGTAVGDAAADVEVVLPWRVRPDDVHLPPLPAAPGPRRRRRPRTPPAAPRARPGERALLLHLPAFGRDLYLQLRRDLRFLSRGFEVEEAGAARRRGRPAELCFYSGRVLGHPGSLVSLSACGAAGGLVGLIQLGQEQVLIQPLNNSQGPFSGREHLIRRKWSLTPSPSAEAQRPEQLCKVLTEKKKPTWGRPSRDWRERRNAIRLTSEHTVETLVVADADMVQYHGAEAAQRFILTVMNMVYNMFQHQSLGIKINIQVTKLVLLRQRPAKLSIGHHGERSLESFCHWQNEEYGGARYLGNNQVPGGKDDPPLVDAAVFVTRTDFCVHKDEPCDTVGIAYLGGVCSAKRKCVLAEDNGLNLAFTIAHELGHNLGMNHDDDHSSCAGRSHIMSGEWVKGRNPSDLSWSSCSRDDLENFLKSKVSTCLLVTDPRSQHTVRLPHKLPGMHYSANEQCQILFGMNATFCRNMEHLMCAGLWCLVEGDTSCKTKLDPPLDGTECGADKWCRAGECVSKTPIPEHVDGDWSPWGAWSMCSRTCGTGARFRQRKCDNPPPGPGGTHCPGASVEHAVCENLPCPKGLPSFRDQQCQAHDRLSPKKKGLLTAVVVDDKPCELYCSPLGKESPLLVADRVLDGTPCGPYETDLCVHGKCQKIGCDGIIGSAAKEDRCGVCSGDGKTCHLVKGDFSHARGTALKDSGKGSINSDWKIELPGEFQIAGTTVRYVRRGLWEKISAKGPTKLPLHLMVLLFHDQDYGIHYEYTVPVNRTAENQSEPEKPQDSLFIWTHSGWEGCSVQCGGGERRTIVSCTRIVNKTTTLVNDSDCPQASRPEPQVRRCNLHPCQSRWVAGPWSPCSATCEKGFQHREVTCVYQLQNGTHVATRPLYCPGPRPAAVQSCEGQDCLSIWEASEWSQCSASCGKGVWKRTVACTNSQGKCDASTRPRAEEACEDYSGCYEWKTGDWSTCSSTCGKGLQSRVVQCMHKVTGRHGSECPALSKPAPYRQCYQEVCNDRINANTITSPRLAALTYKCTRDQWTVYCRVIREKNLCQDMRWYQRCCQTCRDFYANKMRQPPPSS

SEQ ID NO: 7 - DNA, 4342 bp, Homo sapiens
  CDS (148)..(3582)
gctggaggtggcctcccctccgccccagacaagaagaggccctcagccctcccccggtctcagagagccctgagaggaggcccagtccagagctcttcctccgttcccagtccacttctctagggccagtagcagacaccagccagtatgccgaggaaccagggcttctccgagcccgaatactcggccgagtactcagccgagtactccgtcagcctgccctccgaccctgaccgcggggtgggccggacccatgaaatctcggtccggaactcgggctcctgcctgtgcctgcctcgcttcatgcggctgactttcgtgccggagtccttggagaacctctaccagacctacttcaaaaggcagcgccacgagaccctgctggtgctggtggtctttgcagccctctttgactgctacgtggtggtcatgtgtgctgtggtcttctccagcgacaagctggctcccctcgccgtggctggaattggactggtgttggacatcatcctcttcgtgctctgcaaaaaggggctgctcccggaccgggtcacccgcagagtgctgccctacgtgctgtggctgctcataaccgcccagatcttctcctacctgggcctgaacttcgcgcgtgcccacgcggctagtgacacggtgggctggcaggtcttctttgtcttctccttcttcatcacgctgcccctcagcctcagccccatcgtgatcatctccgtggtctcctgtgtggtgcacacgttggtcctgggggtcaccgtggcccagcagcagcaggaggagctcaaggggatgcagctgctgcgggagatcctggccaacgtcttcctctacctgtgcgccatcgctgtgggcatcatgtcctactacatggctgaccgcaagcaccgcaaggccttcctggaggcccgccagtcgctggaggtgaagatgaacctggaagagcagagccagcagcaggagaacctcatgctttccatcctgcccaagcacgtggctgacgagatgctgaaagacatgaagaaagacgagagccagaaggaccagcagcagttcaacaccatgtacatgtaccgtcacgagaacgtcagcatcctctttgccgacatcgtgggctttacccagctgtcttctgcctgcagtgcccaggagcttgtgaagctgctcaacgagctctttgcccgctttgacaagctggcagctaaataccaccagctgcggattaagatcctgggcgactgctactactgcatctgcggcttgcctgactaccgggaggaccacgccgtctgctccatcctcatggggctggccatggtggaggccatctcgtatgtgcgggagaagaccaagactggggtggacatgcgtgtgggggtgcacacgggcaccgtgctggggggcgtcctgggccagaagcgctggcagtacgacgtgtggtcgactgatgtcactgtagccaacaagatggaggccggcggcatccctgggcgcgtgcacatctcccagagcaccatggactgcctgaaaggggagtttgatgtggagccaggcgatgggggcagccgctgtgattacctagaagagaagggtattgaaacctacctcatcattgcctccaagccagaggtgaagaaaacagccacccagaatggcctcaatggctcggccctgcccaatggagcaccagcttcctcaaagtccagctcccctgccctcattgagaccaaggagcccaacgggagtgcccacagcagtgggtccacgtcggagaagcccgaggagcaggatgcccaggccgacaacccctcattccccaacccacgccggaggctgcgcctgcaggacctggctgaccgagtggtggatgcctctgaagatgagcacgagctcaaccagctgctcaacgaggccctgcttgagcgagagtccgcccaagtagtaaagaagagaaacaccttcctcttgtccatgcggttcatggaccccgagatggaaacccgctactcggtggagaaggagaagcagagtggggctgccttcagctgctcctgcgtcgtcctgctctgcacggccctggtcgagatactcatcgacccctggctaatgacaaactatgtgaccttcatggtgggggagattctgctcctcatcctgaccatctgctccctggctgccatctttccccgggcctttcctaagaagcttgtggccttctcaacttggattgaccggacccgctgggccaggaacacctgggccatgctcgccatcttcatcctggtgatggcaaatgtcgtggacatgctcagctgtctccagtactacacgggacccagcaatgcaacggcagggatggagacggagggcagctgcctggagaaccccaagtattacaactatgtggccgtgctgtccctcatcgccaccatcatgctggtgcaggtcagccacatggtgaagctcacgctcatgctgctcgtcgcaggcgccgtggccaccatcaacctctatgcctggcgtcccgtctttgatgaatacgaccacaagcgttttcgggagcacgacttacctatggtggccttagagcagatgcaaggattcaaccctgggctcaatggcactgacaggctgcccctggtgccttccaagtactctatgacggtgatggtgttcctcatgatgctcagcttctactacttctcccgccacgtagaaaaactggcacggacacttttcttgtggaagattgaggtccacgaccagaaggaacgtgtctatgagatgcgacgctggaacgaggccttggtcaccaacatgttgcctgagcacgtggcacgccatttcctggggtccaagaagagagatgaggagctgtatagccagacgtatgatgagattggagtcatgtttgcctccctgcccaactttgctgacttctacacagaggagagcatcaacaatggtggtattgagtgtctgcgtttcctcaatgaaatcatctcagattttgactctctcctggacaatcccaagttccgggtgatcaccaagatcaaaaccattggcagcacgtatatggcggcttcaggagtcacccccgatgtcaacaccaatggctttgccagctccaacaaggaagacaagtccgagagagagcgctggcagcacctggctgacctggccgacttcgcgctggccatgaaggatacgctcaccaacatcaacaaccagtccttcaataacttcatgctgcgcataggcatgaacaaaggcggggttctggctggggtcatcggagcccggaaaccacactacgacatctggggcaatacagtcaatgtagccagcaggatggagtccacgggggtcatgggcaacattcaggtggtagaagaaacccaagtcatcctccgagagtacggcttccgctttgtgaggcgaggccccatctttgtgaaggggaagggggagctgctgaccttcttcttgaaggggcgggataagctagccaccttccccaatggcccctctgtcacactgccccaccaggtggtggacaactcctgaatggcctcgagcctgaaacagtccaaaccggaagggagaatttattttttgaaactgaaggaagtcccgaccttcctggattgaagtgcacactcatggactttaggtttagaaacctcctcagccttcatttgttcgtggatgtgtgagctctgagggtggccctgctattcctctgcgtgcctgtagtgtccccagcataggggtcttaggcatagggctgaacagtccttccagagccctcgttccaatccctgccgtccttgcccctgaggggccctgaccactgtgagcaggagggtggcagagctgggacaaagctgcctttgccgctgggctttccgggactgtggagggagcacaggcggggaagctccacttcagacagggcttggtggggcaggacatggctcccattttgaagggaggtctccatgtggtccgagtgaggtgagacggccctcgtcctggtgttcctgatcatcttgaaaggttcttctggaactcctgtccccttagtcatgagaacagaaagtgcaatatttcctttcacctggcaggggaggggggatttatttctgaaagaaaaatatataaacagatcttctacatttatatttttaatcttctgttaaatacactttccgatattgccttgccttttgagctcttgctacagtcgcctttgctactgctttaagagaatttacaggtattgataaagaacaagactgttttattaaaagctttattcaacttg

SEQ ID NO: 8 - protein, 1144 aa, Homo sapiens
MPRNQGFSEPEYSAEYSAEYSVSLPSDPDRGVGRTHEISVRNSGSCLCLPRFMRLTFVPESLENLYQTYFKRQRHETLLVLVVFAALFDCYVVVMCAVVFSSDKLAPLAVAGIGLVLDIILFVLCKKGLLPDRVTRRVLPYVLWLLITAQIFSYLGLNFARAHAASDTVGWQVFFVFSFFITLPLSLSPIVIISVVSCVVHTLVLGVTVAQQQQEELKGMQLLREILANVFLYLCAIAVGIMSYYMADRKHRKAFLEARQSLEVKMNLEEQSQQQENLMLSILPKHVADEMLKDMKKDESQKDQQQFNTMYMYRHENVSILFADIVGFTQLSSACSAQELVKLLNELFARFDKLAAKYHQLRIKILGDCYYCICGLPDYREDHAVCSILMGLAMVEAISYVREKTKTGVDMRVGVHTGTVLGGVLGQKRWQYDVWSTDVTVANKMEAGGIPGRVHISQSTMDCLKGEFDVEPGDGGSRCDYLEEKGIETYLIIASKPEVKKTATQNGLNGSALPNGAPASSKSSSPALIETKEPNGSAHSSGSTSEKPEEQDAQADNPSFPNPRRRLRLQDLADRVVDASEDEHELNQLLNEALLERESAQVVKKRNTFLLSMRFMDPEMETRYSVEKEKQSGAAFSCSCVVLLCTALVEILIDPWLMTNYVTFMVGEILLLILTICSLAAIFPRAFPKKLVAFSTWIDRTRWARNTWAMLAIFILVMANVVDMLSCLQYYTGPSNATAGMETEGSCLENPKYYNYVAVLSLIATIMLVQVSHMVKLTLMLLVAGAVATINLYAWRPVFDEYDHKRFREHDLPMVALEQMQGFNPGLNGTDRLPLVPSKYSMTVMVFLMMLSFYYFSRHVEKLARTLFLWKIEVHDQKERVYEMRRWNEALVTNMLPEHVARHFLGSKKRDEELYSQTYDEIGVMFASLPNFADFYTEESINNGGIECLRFLNEIISDFDSLLDNPKFRVITKIKTIGSTYMAASGVTPDVNTNGFASSNKEDKSERERWQHLADLADFALAMKDTLTNINNQSFNNFMLRIGMNKGGVLAGVIGARKPHYDIWGNTVNVASRMESTGVMGNIQVVEETQVILREYGFRFVRRGPIFVKGKGELLTFFLKGRDKLATFPNGPSVTLPHQVVDNS

SEQ ID NO: 9 - DNA, 3603 bp, Homo sapiens
  CDS (86)..(2155)
ctcggcctcgggcgcggccgagcgccgcgcgagcaggagcggcggcggcggcggcggcggcgggaggaggcagcgccggcccaagatggcggacctggaggcggtgctggccgacgtgagctacctgatggccatggagaagagcaaggccacgccggccgcgcgcgccagcaagaagatactgctgcccgagcccagcatccgcagtgtcatgcagaagtacctggaggaccggggcgaggtgacctttgagaagatcttttcccagaagctggggtacctgctcttccgagacttctgcctgaaccacctggaggaggccaggcccttggtggaattctatgaggagatcaagaagtacgagaagctggagacggaggaggagcgtgtggcccgcagccgggagatcttcgactcatacatcatgaaggagctgctggcctgctcgcatcccttctcgaagagtgccactgagcatgtccaaggccacctggggaagaagcaggtgcctccggatctcttccagccatacatcgaagagatttgtcaaaacctccgaggggacgtgttccagaaattcattgagagcgataagttcacacggttttgccagtggaagaatgtggagctcaacatccacctgaccatgaatgacttcagcgtgcatcgcatcattgggcgcgggggctttggcgaggtctatgggtgccggaagcgtgacacaggcaagatgtacgccatgaagtgcctggacaaaaagcgcatcaagatgaagcagggggagaccctggccctgaacgagcgcatcatgctctcgctcgtcagcactggggactgcccattcattgtctgcatgtcatacgcgttccacacgccagacaagctcagcttcatcctggacctcatgaacggtggggacctgcactaccacctctcccagcacggggtcttctcagaggctgacatgcgcttctatgcggccgagatcatcctgggcctggagcacatgcacaaccgcttcgtggtctaccgggacctgaagccagccaacatccttctggacgagcatggccacgtgcggatctcggacctgggcctggcctgtgacttctccaagaagaagccccatgccagcgtgggcacccacgggtacatggctccggaggtcctgcagaagggcgtggcctacgacagcagtgccgactggttctctctggggtgcatgctcttcaagttgctgcgggggcacagccccttccggcagcacaagaccaaagacaagcatgagatcgaccgcatgacgctgacgatggccgtggagctgcccgactccttctcccctgaactacactccctgctggaggggttgctgcagagggatgtcaaccggagattgggctgcctgggccgaggggctcaggaggtgaaagagagcccctttttccgctccctggactggcagatggtcttcttgcagaggtaccctcccccgctgatccccccacgaggggaggtgaacgcggccgacgccttcgacattggctccttcgatgaggaggacacaaaaggaatcaagttactggacagtgatcaggagctctaccgcaacttccccctcaccatctcggagcggtggcagcaggaggtggcagagactgtcttcgacaccatcaacgctgagacagaccggctggaggctcgcaagaaagccaagaacaagcagctgggccatgaggaagactacgccctgggcaaggactgcatcatgcatggctacatgtccaagatgggcaacccctttctgacccagtggcagcggcggtacttctacctgttccccaaccgcctcgagtggcggggcgagggcgaggccccgcagagcctgctgaccatggaggagatccagtcggtggaggagacgcagatcaaggagcgcaagtgcctgctcctcaagatccgcggtgggaaacagttcattttgcagtgcgatagcgaccctgagctggtgcagtggaagaaggagctgcgcgacgcctaccgcgaggcccagcagctggtgcagcgggtgcccaagatgaagaacaagccgcgctcgcccgtggtggagctgagcaaggtgccgctggtccagcgcggcagtgccaacggcctctgacccgcccacccgccttttataaacctctaatttattttgtcgaatttttattatttgttttcccgccaagcgaaaaggttttattttgtaattattgtgatttcccgtggccccagcctggcccagctcccccgggaggccccgcttgcctcggctcctgctgcaccaacccagccgctgcccggcgccctctgtcctgacttcaggggctgcccgctcccagtgtcttcctgtgggggaagagcacagccctcccgccccttccccgagggatgatgccacaccaagctgtgccaccctgggctctgtgggctgcacttgtgccatgggactgtgggtggcccatcccccctcaccaggggcaggcacagcacagggatccgacttgaattttcccactgcaccccctcctgctgcagaggggcaggccctgcactgtcctgctccacagtgttggcgagaggaggggcccgttgtctccctggccctcaaggctcccacagtgactcgggctcctgtgcccttattcaggaaaagcctctgtgtcactggctgcctccactcccacttccctgacactgcggggcttggctgagagagtggcattggcagcaggtgctgctaccctccctgctgtcccctcttgccccaacccccagcacccgggctcagggaccacagcaaggcacctgcaggttgggccatactggcctcgcctggcctgaggtctcgctgatgctgggctgggtgcgaccccatctgcccaggacggggccggccaggtgggcgggcagcacagcaaggaggctggctggggcctatcagtgtgccccccatcctggcccatcagtgtacccccgcccagactggccagccccacagcccacgtcctgtcagtgccgccgcctcgcccaccgcatgccccctgtgccagtgctctgcctgtgtgtgtgcactcgtgtcgcgccttctcccccccggggctgggttggcgcaccctcccctcccgtctactcattccccggggcgtttctttgccgatttttgaatgtgattttaaagagtgaaaaatgagactatgcgtttttataaaaaatggtgcctgattcggctgtctcagactctttttgtacctggtgaccccttttcagcttctgctgggctggggcctgatggggagggtctcggtggtaccaggtctcctccaccgccatggcttccaaggtggtctgctcgggcccaggccatcttccaggtggggtgaggcagtgggtcccacttcccctcctacccctcccagctgacagtcctctccacctagtggctgtccagtgcccattcctcaccttttcccggggaggagagagcagcttctgccacttcccaggtaagcaggaggaggtgccaacagtgttaggcctggcacagtgtctgggtgatcgggacct

SEQ ID NO: 10 - protein, 689 aa, Homo sapiens
MADLEAVLADVSYLMAMEKSKATPAARASKKILLPEPSIRSVMQKYLEDRGEVTFEKIFSQKLGYLLFRDFCLNHLEEARPLVEFYEEIKKYEKLETEEERVARSREIFDSYIMKELLACSHPFSKSATEHVQGHLGKKQVPPDLFQPYIEEICQNLRGDVFQKFIESDKFTRFCQWKNVELNIHLTMNDFSVHRIIGRGGFGEVYGCRKRDTGKMYAMKCLDKKRIKMKQGETLALNERIMLSLVSTGDCPFIVCMSYAFHTPDKLSFILDLMNGGDLHYHLSQHGVFSEADMRFYAAEIILGLEHMHNRFVVYRDLKPANILLDEHGHVRISDLGLACDFSKKKPHASVGTHGYMAPEVLQKGVAYDSSADWFSLGCMLFKLLRGHSPFRQHKTKDKHEIDRMTLTMAVELPDSFSPELHSLLEGLLQRDVNRRLGCLGRGAQEVKESPFFRSLDWQMVFLQRYPPPLIPPRGEVNAADAFDIGSFDEEDTKGIKLLDSDQELYRNFPLTISERWQQEVAETVFDTINAETDRLEARKKAKNKQLGHEEDYALGKDCIMHGYMSKMGNPFLTQWQRRYFYLFPNRLEWRGEGEAPQSLLTMEEIQSVEETQIKERKCLLLKIRGGKQFILQCDSDPELVQWKKELRDAYREAQQLVQRVPKMKNKPRSPVVELSKVPLVQRGSANGL

SEQ ID NO: 11 - DNA, 2031 bp, Homo sapiens
  CDS (255)..(518)
ggcacgagggcgagctgaggtggaggcaggctgcggcagacggcgacagtggcggcggcgccatggcagggcttgcaggatccctgctgccttggtgatcccgggctgacagccagagagcacagcggctcagctcctggagagtgagggttgaagaaagcggagggcagccgcctgcgcccgctggctcccattaggtcggttcctgcagcggtgcccggcagccttggtgaaggccctgcccggcagagatcatgtattgcctccagtggctgctgcccgtcctcctcatccccaagcccctcaaccccgccctgtggttcagccactccatgttcatgggcttctacctgctcagcttcctcctggaacggaagccttgcacaatttgtgccttggttttcctggcagccctgttccttatctgctatagctgctggggaaactgtttcctgtaccactgctccgattccccgcttccagaatcggcgcatgatcccggcgttgtgggcacctaacggcctgccctgttagctttccaaggaagcagaagacgggaggggaggcattgacataggtcataaagcattggagtttcaaatcccgcagcctcgcgggtgtcacattcctgacggcgcctttttggcctgtgatgttttatccttacaatgtgaataatggcactgaccggtgcttttattgtaaagtcctatagtcgtgggtggtcttgtggttgtgtgtgttctgtccccatctaggtcctggctggccgcatgaccacccctctcgcctcattactgtgaggagtctgggtccatcctggtcagctgccccaatgtgacctggggcagataaaatgccagtctcattgtcacctctgtgacccctccttgtcagggtctccttccttcccagaatgttactgactcctcagtccctcttctggtttccctttatttctcttctacccttttccttttttggggagtacctgtccaagacagggctcatttttgcacttatctcgaatttgaagagattgctgacgcccgagagcctcgctttttcatccttctttccttgttcagcaggctagacagaaacatgtcttgactgttagttgtccacaaatcttcagtattttctccacttcatttttaagaaaggaagcaacagatagatgttgctctttcacctgggtgtctgggctcaagctttcccgcccagcctcacttcctttgcccttcctcctgcctttctcaactgtcccaaggagggggcctcattgtgtctcccgtgcatgctctgcagcattgaagtatggtgtgttcacgtagttctagcagtccccagctgagtgagtgggagagtacctgtgtgtttcgtaacggccttgatccccttgatagatgtttggatattttttggtgtgccctgtgtgtgtgtgtgtgtacaaatacatgtgtatattccttttaaagaagctttatcgaacgtggtctgattttgaggtttagcaatagctagctatatatggtaggtgccgctacagtttttatttagcatggggattgcagagtgaccagcacactggactccgaggtggttcagacaagacagaggggagcagtggccatcatcctcccgccaggagcttcttcgttcctgcgcatatagactgtacgttatgaagaatacccaggaagactttgtgactgtcacttgctgctttttctgcgcttcagtaacaagtgttggcaaacgagactttctcctggcccctgcctgctggagatcagcatgcctgtcctttcagtctgatccatccatctctctcttgcctgaggggaaagagagatgggccaggcagagaacagaactggaggcagtccatctagggaatgggactgtgaggccatacttgtgaaacgtctggactgctattctagagcttttatttggtgtgttcgttgcacagctgtttgaaatgtttaataaagctttataaactttaaaaaaaaaaaaaaaaaa

SEQ ID NO: 12 - protein, 87 aa, Homo sapiens
MYCLQWLLPVLLIPKPLNPALWFSHSMFMGFYLLSFLLERKPCTICALVFLAALFLICYSCWGNCFLYHCSDSPLPESAHDPGVVGT

SEQ ID NO: 13 - DNA, 3578 bp, Homo sapiens
  CDS (268)..(1389)
cgccggcggcggcggagactccggggctgcggcgccgcccgccccgcccgcagagtccggctgccgcgcatcgtccgcagacgccgccaccgccatgggctcctgaggctagcttgtcactttctgcaaaggtttccctcagggagcctcctgctgccaggcaccatgacagtgaggggggatgtgctggccccggatccagcgtcgcccacgaccgcagcagcctcgcccagcgtctccgtgatccccgagggcagccccactgccatggagcagcctgtgttcctgatgacaactgccgctcaggccatctctggcttcttcgtgtggacggccctgctcatcacatgccaccagatctacatgcacctgcgctgctacagctgccccaacgagcagcgctacatcgtgcgcatcctcttcatcgtgcccatctacgcctttgactcctggctcagcctcctcttcttcaccaacgaccagtactacgtgtacttcggcaccgtccgcgactgctatgaggccttggtcatctataatttcctgagcctgtgctatgagtacctaggaggagaaagttccatcatgtcggagatcagaggaaaacccattgagtccagctgtatgtatggcacctgctgcctctggggaaagacttattccatcggatttctgaggttctgcaaacaggccaccctgcagttctgtgtggtgaagccactcatggcggtcagcactgtggtcctccaggccttcggcaagtaccgggatggggactttgacgtcaccagtggctacctctacgtgaccatcatctacaacatctccgtcagcctggccctctacgccctcttcctcttctacttcgccacccgggagctgctcagcccctacagccccgtcctcaagttcttcatggtcaagtccgtcatctttctttccttctggcaaggcatgctcctggccatcctggagaagtgtggggccatccccaaaatccactcggcccgcgtgtcggtgggcgagggcaccgtggctgccggctaccaggacttcatcatctgtgtggagatgttctttgcagccctggccctgcggcacgccttcacctacaaggtctatgctgacaagaggctggacgcacaaggccgctgtgcccccatgaagagcatctccagcagcctcaaggagaccatgaacccgcacgacatcgtgcaggacgccatccacaacttctcacctgcctaccagcagtacacgcagcagtccaccctggagcctgggcccacctggcgtggtggcgcccacggcctctcccgctcccacagcctcagtggcgcccgcgacaacgagaagactctcctgctcagctctgatgatgaattctaggtgcgggctgcagtggcggaagtgctggcgccatagccacggtcaggctgtgccccacctccagcctcaccaccaggccaggaggcagctggcacagtgctcacgccgcctttatttattggaccagaaacactcacatgtcacttccagaggaacgggggacagccaggctcgcccatgggccttcaggaatatttatacatggcccagcctgcactgcccgggcgagggcagaggacactgggagcaaggcttatgcccctgctgcccgtcctgtgctgggggcatgctgggaccagccgcacccaggccccaatgcttgtgtgtggaccagcggctgcagccttctagcccctcctccccgcgagactctcaggctgaggtcggcaagccgtggctcccccacacaccgtgcaataccctgtctgacctgggctcttcccgcctgcatccctcccctgtccacctttgtccagtgctagattcacctcaccccgggcaggagtggggatgtgggcgctctgtggtcctcccctcctgacccaggcctctgtggcatgctgcaaggatcagagccagacaccaggagtcacaggccccacccaggaagggcattcagggcccctgggcaccgcttctgttgaagcaggggcttctgggcccctgggtatccccacctgtcgtggccacacctctgcctgcctcatgccccttccccctggcctaccaaggacagcccacagcccgcactgccggctcacttgggtccttcctcgatagctttgggcagagcccttgcttcctggctgcttcagggctcaggggctcccagccctccttcccaggctgatgctgggtcctctctctctttggggcttctccctcccgtttcaggggaaaggtctgagtctccacgtttcagaccagcttctgggggaaggcagtccggcagggagaccgggaggggtggccacacagtggggagctgggaggtggggggaatggtcccagactcctctcggggcccctatccacacagggcctggtgttctaccccatctggcccctggcccatctcttctgtgccttagtcacatatgaaagcgcccctccctggctccccatctgtcccacacgctccctggggctcttagttcagctgctggcactcgcaggatcctgcagtgctgggcccagagcccttggacaggcctcaggagtggtcaggaccaccaagcccctcctctccccctccacacctctagacctggggcctccggaacccccagcaggctgggcttatactagctcctgacttaggaagagcctcgtgtcacaacacgtgtccctacaggcaaagtgtcctggcatttaaaacccagattatccctgggtttgggctgcagtcacctggagaagctggtagggtaagggagagggaccctgccggtgttcactggggattctttcttttggtccttcctggaatgaacaggttccctccctgccacctgtgaggagagttggggcccagccgtcttcctggcctccttcctttcctcgtggcagaggcctgcatgtgggtgccagaggccagctctccccctccatcttgggggggcggagcagttgggcccaagctgcccgggagggtgggtgcagacacaggctgaggaccagccctggccctgccccgccatctgctttcaccaagctgtctctccaccgtggcttcccttctccctccaggccaaagtgctgctgattcccactcccttggttttcgcctgcccagcgttgctgtttgcgtggagggtggggggagctcagtggcagggaatcagcggtccgtggggtcgtggggacgggaacatgtgcccgaccgctccatcccctcctcctccttaggatgcataacctaccttgtctttttttttttaaattttctttccaggtagagtagctctttgtacataaagaatacttgaaaaattaattgtatgatgtatgagaagacagagtctcctagttttgtatcttgttgtatgactgccatgagttccaccagaaagccactctattttggtccctgtgacattttaaatgcgtgacagaagtgagcaaataaagtgaggaagaaatctatatatgagataatatagattgtattgaaaaaaaaaaaaaaaaaa

SEQ ID NO: 14 - protein, 373 aa, Homo sapiens
MEQPVFLMTTAAQAISGFFVWTALLITCHQIYMHLRCYSCPNEQRYIVRILFIVPIYAFDSWLSLLFFTNDQYYVYFGTVRDCYEALVIYNFLSLCYEYLGGESSIMSEIRGKPIESSCMYGTCCLWGKTYSIGFLRFCKQATLQFCVVKPLMAVSTVVLQAFGKYRDGDFDVTSGYLYVTIIYNISVSLALYALFLFYFATRELLSPYSPVLKFFMVKSVIFLSFWQGMLLAILEKCGAIPKIHSARVSVGEGTVAAGYQDFIICVEMFFAALALRHAFTYKVYADKRLDAQGRCAPMKSISSSLKETMNPHDIVQDAIHNFSPAYQQYTQQSTLEPGPTWRGGAHGLSRSHSLSGARDNEKTLLLSSDDEF

SEQ ID NO: 15 - DNA, 1332 bp, Homo sapiens
  CDS (41)..(751)
ccggcccgcgccccgcaggccgcccgccgcccgcgccgccatgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactgaggccccgcagctctggccacagggacctctgcagtgccccctaagtgacccggacacttccgagggggccatcaccgcctgtgtatataacgtttccggtattactctgctacacgtagcctttttacttttggggttttgtttttgttctgaactttcctgttaccttttcagggctgacgtcacatgtaggtggcgtgtatgagtggagacgggcctgggtcttggggactggagggcaggggtccttctgccctggggtcccagggtgctctgcctgctcagccaggcctctcctgggagccactcgcccagagactcagcttggccaacttggggggctgtgtccacccagcccgcccgtcctgtgggctgcacagctcaccttgttccctcctgccccggttcgagagccgagtctgtgggcactctctgccttcatgcacctgtcctttctaacacgtcgccttcaactgtaatcacaacatcctgactccgtcatttaataaagaaggaacatcaggcatgctaaaaaaaaaaaaaaaaaaaaaaaaaaaaaaaaaaaaaaaaaaaaaaaaaaa

SEQ ID NO: 16 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 17 - DNA, 1246 bp, Homo sapiens
  CDS (112)..(798)
gaccagcctacagccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcaaaaaaaaaaaaaaa

SEQ ID NO: 18 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 19 - DNA, 1846 bp, Homo sapiens
  CDS (340)..(969)
aaaagtgcctttgttggcctgggctcaggaatccagagaaactggtcaggaggaggccccagtgacaaaaacccctccctctgcccccgcccctctgccagagccatataactgctcaacctgtccccgagagagagtgccctggcagctgtcggctggaaggaactggtctgctcacacttgctggcttgcgcatcaggactggctttatctcctgactcacggtgcaaaggtgcactctgcgaacgttaagtccgtccccagcgcttggaatcctacggcccccacagccggatcccctcagccttccaggtcctcaactcccgtggacgctgaacaatggcctccatggggctacaggtaatgggcatcgcgctggccgtcctgggctggctggccgtcatgctgtgctgcgcgctgcccatgtggcgcgtgacggccttcatcggcagcaacattgtcacctcgcagaccatctgggagggcctatggatgaactgcgtggtgcagagcaccggccagatgcagtgcaaggtgtacgactcgctgctggcactgccgcaggacctgcaggcggcccgcgccctcgtcatcatcagcatcatcgtggctgctctgggcgtgctgctgtccgtggtggggggcaagtgtaccaactgcctggaggatgaaagcgccaaggccaagaccatgatcgtggcgggcgtggtgttcctgttggccggccttatggtgatagtgccggtgtcctggacggcccacaacatcatccaagacttctacaatccgctggtggcctccgggcagaagcgggagatgggtgcctcgctctacgtcggctgggccgcctccggcctgctgctccttggcggggggctgctttgctgcaactgtccaccccgcacagacaagccttactccgccaagtattctgctgcccgctctgctgctgccagcaactacgtgtaaggtgccacggctccactctgttcctctctgctttgttcttccctggactgagctcagcgcaggctgtgaccccaggagggccctgccacgggccactggctgctggggactggggactgggcagagactgagccaggcaggaaggcagcagccttcagcctctctggcccactcggacaacttcccaaggccgcctcctgctagcaagaacagagtccaccctcctctggatattggggagggacggaagtgacagggtgtggtggtggagtggggagctggcttctgctggccaggatagcttaaccctgactttgggatctgcctgcatcggcgttggccactgtccccatttacattttccccactctgtctgcctgcatctcctctgttccgggtaggccttgatatcacctctgggactgtgccttgctcaccgaaacccgcgcccaggagtatggctgaggccttgcccacccacctgcctgggaagtgcagagtggatggacgggtttagaggggaggggcgaaggtgctgtaaacaggtttgggcagtggtgggggagggggccagagaggcggctcaggttgcccagctctgtggcctcaggactctctgcctcacccgcttcagcccagggcccctggagactgatcccctctgagtcctctgccccttccaaggacactaatgagcctgggagggtggcagggaggaggggacagcttcacccttggaagtcctggggtttttcctcttccttctttgtggtttctgttttgtaatttaagaagagctattcatcactgtaattattattattttctacaataaatgggacctgtgcacaggaaaaaaaaaaaaaaaaaaaaaaaa

SEQ ID NO: 20 - protein, 209 aa, Homo sapiens
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 21 - DNA, 1225 bp, Homo sapiens
  CDS (237)..(962)
gcggctctctgatccagcccgggagaggaccgagctggaggagctgggtgtggggtgcgttgggctggtggggaggcctagtttgggtgcaagtaggtctgattgagcttgtgttgtgctgaagggacagccctgggtctaggggagagagtccctgagtgtgagacccgccttccccggtcccagcccctcccagttcccccagggacggccacttcctggtccccgacgcaaccatggctgaagaacaaccgcaggtcgaattgttcgtgaaggctggcagtgatggggccaagattgggaactgcccattctcccagagactgttcatggtactgtggctcaagggagtcaccttcaatgttaccaccgttgacaccaaaaggcggaccgagacagtgcagaagctgtgcccaggggggcagctcccattcctgctgtatggcactgaagtgcacacagacaccaacaagattgaggaatttctggaggcagtgctgtgccctcccaggtaccccaagctggcagctctgaaccctgagtccaacacagctgggctggacatatttgccaaattttctgcctacatcaagaattcaaacccagcactcaatgacaatctggagaagggactcctgaaagccctgaaggttttagacaattacttaacatcccccctcccagaagaagtggatgaaaccagtgctgaagatgaaggtgtctctcagaggaagtttttggatggcaacgagctcaccctggctgactgcaacctgttgccaaagttacacatagtacaggtggtgtgtaagaagtaccggggattcaccatccccgaggccttccggggagtgcatcggtacttgagcaatgcctacgcccgggaagaattcgcttccacctgtccagatgatgaggagatcgagctcgcctatgagcaagtggcaaaggccctcaaataagcccctcctgggactccctcaaccccctccattttctccacaaaggccctggtggtttccacattgctacccaatggacacactccaaaatggccagtgggcagggaatcctggagcacttgttccgggatggtgtggtggaagaggggatgagggaaagaaatggggggcctgggtcagatttttattgtggggtggggtgagtaggacaacatatttcagtaataaaatacagaataaaaatcaagtgtttttaaaaaaaa

SEQ ID NO: 22 - protein, 241 aa, Homo sapiens
MAEEQPQVELFVKAGSDGAKIGNCPFSQRLFMVLWLKGVTFNVTTVDTKRRTETVQKLCPGGQLPFLLYGTEVHTDTNKIEEFLEAVLCPPRYPKLAALNPESNTAGLDIFAKFSAYIKNSNPALNDNLEKGLLKALKVLDNYLTSPLPEEVDETSAEDEGVSQRKFLDGNELTLADCNLLPKLHIVQVVCKKYRGFTIPEAFRGVHRYLSNAYAREEFASTCPDDEEIELAYEQVAKALK

SEQ ID NO: 23 - DNA, 3438 bp, Homo sapiens
  CDS (74)..(2560)
gcgcccgcgcctcgggccgtcgggagcggagcctcctcgggaccaggacttcagggccacaggtgctgccaagatgctccagggcacctgctccgtgctcctgctctggggaatcctgggggccatccaggcccagcagcaggaggtcatctcgccggacactaccgagagaaacaacaactgcccagagaagaccgactgccccatccacgtgtacttcgtgctggacacctcggagagcgtcaccatgcagtcccccacggacatcctgctcttccacatgaagcagttcgtgccgcagttcatcagccagctgcagaacgagttctacctggaccaggtggcgctgagctggcgctacggcggcctgcacttctctgaccaggtggaggtgttcagcccaccgggcagcgaccgggcctccttcatcaagaacctgcagggcatcagctccttccgccgcggcaccttcaccgactgcgcgctggccaacatgacggagcagatccggcaggaccgcagcaagggcaccgtccacttcgccgtggtcatcaccgacggccacgtcaccggcagcccctgcgggggcatcaagctgcaggccgagcgggcccgcgaggagggcatccggctcttcgccgtggcccccaaccagaacctgaaggagcagggcctgcgggacatcgccagcacgccgcacgagctctaccgcaacgactacgccaccatgctgcccgactccaccgagatcaaccaggacaccatcaaccgcatcatcaaggtcatgaaacacgaagcctacggagagtgctacaaggtgagctgcctggaaatccctgggccctctgggcccaagggctaccgtggacagaagggtgccaagggcaacatgggtgagccgggagagcctggccagaagggaagacagggagacccgggcatcgaaggccccattggattcccaggacccaagggcgttcctggcttcaaaggagagaagggtgaatttggagccgacggtcgcaagggggcccctggcctggctggcaagaacgggaccgatggacagaagggcaagctggggcgcatcggacctcctggctgcaagggagaccctggaaaccggggccccgacggttacccgggggaagcagggagtccaggggagcgaggagaccaaggcggcaagggggaccctggccgcccaggacgcagagggcccccgggagaaatcggggccaagggaagcaaggggtatcaaggcaacaatggagccccaggaagtcctggtgtgaaaggagccaagggcgggcctgggccccgcggacccaaaggcgagccggggcgcaggggagaccccggcaccaagggcagcccaggcagcgatggccccaagggggagaagggggaccctggccctgagggcccccgcggcctggctggagaggttggcaacaaaggagccaagggagaccgaggcttgcctggacccagaggcccccagggagctcttggggagcccggaaagcagggatctcggggagaccccggtgatgcaggaccccgtggagactcaggacagccaggccccaagggagaccccggcaggcctggattcagctacccaggaccccgaggagcacccggagaaaaaggcgagcccggcccacgcggccccgagggaggccgaggcgactttggcttgaaaggagaacctgggaggaaaggagagaaaggagagcctgcggatcctggtccccctggtgagccaggccctcgggggccaagaggagtcccaggacccgagggtgagcccggcccccctggagaccccggtctcacggagtgtgacgtcatgacctacgtgagggagacctgcgggtgctgcgactgtgagaagcgctgtggcgccctggacgtggtcttcgtcatcgacagctccgagagcattgggtacaccaacttcacactggagaagaacttcgtcatcaacgtggtcaacaggctgggtgccatcgctaaggaccccaagtccgagacagggacgcgtgtgggcgtggtgcagtacagccacgagggcacctttgaggccatccagctggacgacgaacatatcgactccctgtcgagcttcaaggaggctgtcaagaacctcgagtggattgcgggcggcacctggacaccctcagccctcaagtttgcctacgaccgcctcatcaaggagagccggcgccagaagacacgtgtgtttgcggtggtcatcacggacgggcgccacgaccctcgggacgatgacctcaacttgcgggcgctgtgcgaccgcgacgtcacagtgacggccatcggcatcggggacatgttccacgagaagcacgagagtgaaaacctctactccatcgcctgcgacaagccacagcaggtgcgcaacatgacgctgttctccgacctggtcgctgagaagttcatcgatgacatggaggacgtcctctgcccggaccctcagatcgtgtgcccagaccttccctgccaaacaggtttggacggagctgttttgtgctgaaaggttttctcggggtccgtggtgtcccccaaaggtgccaccgtgcgggtctcctagctccctgccagcttcctgtccctgtgctcactgcccccacgcctcctgccaaggccgagccacacacccgctccacctgcatttcctctaccgactcgccagcccaaatgccgctcttcactctggcctcgctgagcggctgcccgaggaggagctctaggccgacgcccaccgcaggccttacagtcgtctctggacgctcccttgcagatgcaccgtggcctggcggcgagcccccggtcaccttcctccgcacggaagaggggccggacgccaccttccccaggaccattcccctgatccaacagttgctaaacgccacggagctcacgcaggacccggccgcctactcccagctggtggccgtgctggtctacaccgccgagcgggccaagttcgccaccggggtagagcggcaggactggatggagctgttcattgacacctttaagctggtgcacagggacatcgtgggggaccccgagaccgcgctggccctctgctaaagcccgggcacccgcccagccgggctgggccctccctgccacactagcttcccagggctgcccccgacaggctggctctcagtggaggccgagagatctggaatcggggtcagcggggctacagtccttccaggggctctggggcagctcccagcctcttcccatgctggtggccaccgtgtcccttgctgcggctgcatcttccagtctctcctccgtcttccagtggccgctctctttataagaaccctggtcattgaatttaaggcccaccccaagtccagaatgacctcgcaagacccttaactcactccc

SEQ ID NO: 24 - protein, 828 aa, Homo sapiens
MLQGTCSVLLLWGILGAIQAQQQEVISPDTTERNNNCPEKTDCPIHVYFVLDTSESVTMQSPTDILLFHMKQFVPQFISQLQNEFYLDQVALSWRYGGLHFSDQVEVFSPPGSDRASFIKNLQGISSFRRGTFTDCALANMTEQIRQDRSKGTVHFAVVITDGHVTGSPCGGIKLQAERAREEGIRLFAVAPNQNLKEQGLRDIASTPHELYRNDYATMLPDSTEINQDTINRIIKVMKHEAYGECYKVSCLEIPGPSGPKGYRGQKGAKGNMGEPGEPGQKGRQGDPGIEGPIGFPGPKGVPGFKGEKGEFGADGRKGAPGLAGKNGTDGQKGKLGRIGPPGCKGDPGNRGPDGYPGEAGSPGERGDQGGKGDPGRPGRRGPPGEIGAKGSKGYQGNNGAPGSPGVKGAKGGPGPRGPKGEPGRRGDPGTKGSPGSDGPKGEKGDPGPEGPRGLAGEVGNKGAKGDRGLPGPRGPQGALGEPGKQGSRGDPGDAGPRGDSGQPGPKGDPGRPGFSYPGPRGAPGEKGEPGPRGPEGGRGDFGLKGEPGRKGEKGEPADPGPPGEPGPRGPRGVPGPEGEPGPPGDPGLTECDVMTYVRETCGCCDCEKRCGALDVVFVIDSSESIGYTNFTLEKNFVINVVNRLGAIAKDPKSETGTRVGVVQYSHEGTFEAIQLDDEHIDSLSSFKEAVKNLEWIAGGTWTPSALKFAYDRLIKESRRQKTRVFAVVITDGRHDPRDDDLNLRALCDRDVTVTAIGIGDMFHEKHESENLYSIACDKPQQVRNMTLFSDLVAEKFIDDMEDVLCPDPQIVCPDLPCQTGLDGAVLC

SEQ ID NO: 25 - DNA, 3389 bp, Homo sapiens
  CDS (82)..(2202)
agtgcctccctccagactcgggagggtcgagggggcgcgggagagagcgcgggcggccgccggggctggtcgcctgcagggatgggggacgagcggccccactactacgggaaacacggaacgccacagaagtatgatcccactttcaaaggacccatttacaataggggctgcacggatatcatatgctgtgtgttcctgctcctggccattgtgggctacgtggctgtaggcatcatagcctggactcatggagaccctcgaaaggtgatctaccccactgatagccggggcgagttctgcgggcagaagggcacaaaaaacgagaacaaaccctatctgttttatttcaacattgtgaaatgtgccagccccctggttctgctggaattccaatgtcccactccccagatctgcgtggaaaaatgccccgaccgctacctcacgtacctgaatgctcgcagctcccgggactttgagtactataagcagttctgtgttcctggcttcaagaacaataaaggagtggctgaggtgcttcgagatggtgactgccctgctgtcctcatccccagcaaacccttggcccggagatgcttccccgctatccacgcctacaagggtgtcctgatggtgggcaatgagacgacctatgaggatgggcatggctcccggaaaaacatcacagacctggtggagggcgccaagaaagccaatggagtcctagaggcgcggcaactcgccatgcgcatatttgaagattacaccgtctcttggtactggattatcataggcctggtcattgccatggcgatgagcctcctgttcatcatcctgcttcgcttcctggctggtattatggtctgggtgatgatcatcatggtgattctggtgctgggctacggaatatttcactgctacatggagtactcccgactgcgtggtgaggccggctctgatgtctctttggtggacctcggctttcagacggatttccgggtgtacctgcacttacggcagacctggttggcctttatgatcattctgagtatccttgaagtcattatcatcttgctgctcatctttctccggaagagaattctcatcgcgattgcactcatcaaagaagccagcagggctgtgggatacgtcatgtgctccttgctctacccactggtcaccttcttcttgctgtgcctctgcatcgcctactgggccagcactgctgtcttcctgtccacttccaacgaagcggtctataagatctttgatgacagcccctgcccatttactgcgaaaacctgcaacccagagaccttcccctcctccaatgagtcccgccaatgccccaatgcccgttgccagttcgccttctacggtggtgagtcgggctaccaccgggccctgctgggcctgcagatcttcaatgccttcatgttcttctggttggccaacttcgtgctggcgctgggccaggtcacgctggccggggcctttgcctcctactactgggccctgcgcaagccggacgacctgccggccttcccgctcttctctgcctttggccgggcgctcaggtaccacacaggctccctggcctttggcgcgctcatcctggccattgtgcagatcatccgtgtgatactcgagtacctggatcagcggctgaaagctgcagagaacaagtttgccaagtgcctcatgacctgtctcaaatgctgcttctggtgcctggagaagttcatcaaattccttaataggaatgcctacatcatgattgccatctacggcaccaatttctgcacctcggccaggaatgccttcttcctgctcatgagaaacatcatcagagtggctgtcctggataaagttactgacttcctcttcctgttgggcaaacttctgatcgttggtagtgtggggatcctggctttcttcttcttcacccaccgtatcaggatcgtgcaggatacagcaccacccctcaattattactgggttcctatactgacggtgatcgttggctcctacttgattgcacacggtttcttcagcgtctatggcatgtgtgtggacacgctgttcctctgcttcttggaggacctggagaggaatgacggctcggccgagaggccttacttcatgtcttccaccctcaagaaacccttgaacaagaccaacaagaaggcagcggagtcctgaaggccccgtgctccccacctctcaaggagtctcatgccgcagggtgctcagtagctgggtctgttcccccagccccttgggctcacctgaagtcctatcactgccgctctgcccctccccatgagccagatcccaccagtttctggacgtggagagtctggggcatctccttcttatgccaaggggcgcttggagttttcatggctgcccctccagactgcgagaaacaagtaaaaacccattggggcctcttgatgtctgggatggcacgtggcccgacctccacaagctccctcatgcttcctgtcccccgcttacacgacaacgggccagaccacgggaaggacggtgtttgtgtctgagggagctgctggccacagtgaacacccacgtttattcctgcctgctccggccaggactgaaccccttctccacacctgaacagttggctcaagggccaccagaagcatttctttattattattattttttaacctggacatgcattaaagggtctattagctttctttccgtctgtctcaacagctgagatggggccgccaaggagtgccttccttttgctccctcctagctgggagtgacgggtgggagtgtgtgtgcccaggtgggggtgtctcctggctgggaaggagggaaagggagggagagttttgcgggggttggcagtggagagcaggctggagaggagatggctaatagctgtttaatggaaacctgctgggctggagggagttaggctgaatttcccgacttcctctgccagttattgacacagctctctttgtaagagaggaaagaaactaaacccacccaagggatgatttcagggggagaggtggagggcagatgtcctgggcaaaccgggcccctctgcccacacacctcacttgatccttttgccaaacttgtcaaactcaggggaactggcttcccagttgcccctttgccatattccaagtccccctcagacttcatgtctctgctcatcagcactgtcccaggatcctggagagggagaacccctggccccaggggaaagaggggggggtctcccgtttcctgtgcctgcaccagccctgcccccattgcgtctgcacacccctgcgtgtaactgcattccaaccactaataaagtgcctattgtacaggtaaaaaaaaaaaaaaaa

SEQ ID NO: 26 - protein, 706 aa, Homo sapiens
MGDERPHYYGKHGTPQKYDPTFKGPIYNRGCTDIICCVFLLLAIVGYVAVGIIAWTHGDPRKVIYPTDSRGEFCGQKGTKNENKPYLFYFNIVKCASPLVLLEFQCPTPQICVEKCPDRYLTYLNARSSRDFEYYKQFCVPGFKNNKGVAEVLRDGDCPAVLIPSKPLARRCFPAIHAYKGVLMVGNETTYEDGHGSRKNITDLVEGAKKANGVLEARQLAMRIFEDYTVSWYWIIIGLVIAMAMSLLFIILLRFLAGIMVWVMIIMVILVLGYGIFHCYMEYSRLRGEAGSDVSLVDLGFQTDFRVYLHLRQTWLAFMIILSILEVIIILLLIFLRKRILIAIALIKEASRAVGYVMCSLLYPLVTFFLLCLCIAYWASTAVFLSTSNEAVYKIFDDSPCPFTAKTCNPETFPSSNESRQCPNARCQFAFYGGESGYHRALLGLQIFNAFMFFWLANFVLALGQVTLAGAFASYYWALRKPDDLPAFPLFSAFGRALRYHTGSLAFGALILAIVQIIRVILEYLDQRLKAAENKFAKCLMTCLKCCFWCLEKFIKFLNRNAYIMIAIYGTNFCTSARNAFFLLMRNIIRVAVLDKVTDFLFLLGKLLIVGSVGILAFFFFTHRIRIVQDTAPPLNYYWVPILTVIVGSYLIAHGFFSVYGMCVDTLFLCFLEDLERNDGSAERPYFMSSTLKKPLNKTNKKAAES

SEQ ID NO: 27 - DNA, 2409 bp, Homo sapiens
  CDS (1)..(2313)
atgcggggcgtgtggccgcccccggtgtccgccctgctgtcggcgctggggatgtcgacgtacaagcgggccacgctggacgaggaggacctggtggactcgctctccgagggcgacgcataccccaacggcctgcaggtgaacttccacagcccccggagtggccagaggtgctgggctgcacggacccaggtggagaagcggctggtggtgttggtggtacttctggcggcaggactggtggcctgcttggcagcactgggcatccagtaccagacaagatccccctctgtgtgcctgagcgaagcttgtgtctcagtgaccagctccatcttgagctccatggaccccacagtggacccctgccatgacttcttcagctacgcctgtgggggctggatcaaggccaacccagtccctgatggccactcacgctgggggaccttcagcaacctctgggaacacaaccaagcaatcatcaagcacctcctcgaaaactccacggccagcgtgagcgaggcagagagaaaggcgcaagtatactaccgtgcgtgcatgaacgagaccaggatcgaggagctcagggccaaacctctaatggagttgattgagaggctcgggggctggaacatcacaggtccctgggccaaggacaacttccaggacaccctgcaggtggtcaccgcccactaccgcacctcacccttcttctctgtctatgtcagtgccgattccaagaactccaacagcaacgtgatccaggtggaccagtctggcctgggcttgccctcgagagactattacctgaacaaaactgaaaacgagaaggtgctgaccggatatctgaactacatggtccagctggggaagctgctgggcggcggggacgaggaggccatccggccccagatgcagcagatcttggactttgagacggcactggccaacatcaccatcccacaggagaagcgccgtgatgaggagctcatctaccacaaagtgacggcagccgagctgcagaccttggcacccgccatcaactggttgccttttctcaacaccatcttctaccccgtggagatcaatgaatccgagcctattgtggtctatgacaaggaataccttgagcagatctccactctcatcaacaccaccgacagatgcctgctcaacaactacatgatctggaacctggtgcggaaaacaagctccttccttgaccagcgctttcaggacgccgatgagaagttcatggaagtcatgtacgggaccaagaagacctgtcttcctcgctggaagttttgcgtgagtgacacagaaaacaacctgggctttgcgttgggccccatgtttgtcaaagcaaccttcgccgaggacagcaagagcatagccaccgagatcatcctggagattaagaaggcatttgaggaaagcctgagcaccctgaagtggatggatgaggaaacccgaaaatcagccaaggaaaaggccgatgccatctacaacatgataggataccccaacttcatcatggatcccaaggagctggacaaagtgtttaatgactacactgcagttccagacctctactttgaaaatgccatgcggtttttcaacttctcatggagggtcactgccgatcagctcaggaaagcccccaacagagatcagtggagcatgaccccgcccatggtgaacgcctactactcgcccaccaagaatgagattgtgtttccggccgggatcctgcaggcaccattctacacacgctcctcacccaaggccttaaactttggtggcataggtgtcgtcgtgggccatgagctgactcatgcttttgatgatcaaggacgggagtatgacaaggacgggaacctccggccatggtggaagaactcatccgtggaggccttcaagcgtcagaccgagtgcatggtagagcagtacagcaactacagcgtgaacggggagccggtgaacgggcggcacaccctgggggagaacatcgccgacaacgggggtctcaaggcggcctatcgggcttaccagaactgggtgaagaagaacggggctgagcactcgctccccaccctgggcctcaccaataaccagctcttcttcctgggctttgcacaggtctggtgctccgtccgcacacctgagagctcccacgaaggcctcatcaccgatccccacagcccctctcgcttccgggtcatcggctccctctccaattccaaggagttctcagaacacttccgctgcccacctggctcacccatgaacccgcctcacaagtgcgaagtctggtaaggacgaagcggagagagccaagacggaggaggggaaggggctgaggacgagacccccatccagcctccagggcattgctcagcccgcttggccacc

SEQ ID NO: 28 - protein, 770 aa, Homo sapiens
MRGVWPPPVSALLSALGMSTYKRATLDEEDLVDSLSEGDAYPNGLQVNFHSPRSGQRCWAARTQVEKRLVVLVVLLAAGLVACLAALGIQYQTRSPSVCLSEACVSVTSSILSSMDPTVDPCHDFFSYACGGWIKANPVPDGHSRWGTFSNLWEHNQAIIKHLLENSTASVSEAERKAQVYYRACMNETRIEELRAKPLMELIERLGGWNITGPWAKDNFQDTLQVVTAHYRTSPFFSVYVSADSKNSNSNVIQVDQSGLGLPSRDYYLNKTENEKVLTGYLNYMVQLGKLLGGGDEEAIRPQMQQILDFETALANITIPQEKRRDEELIYHKVTAAELQTLAPAINWLPFLNTIFYPVEINESEPIVVYDKEYLEQISTLINTTDRCLLNNYMIWNLVRKTSSFLDQRFQDADEKFMEVMYGTKKTCLPRWKFCVSDTENNLGFALGPMFVKATFAEDSKSIATEIILEIKKAFEESLSTLKWMDEETRKSAKEKADAIYNMIGYPNFIMDPKELDKVFNDYTAVPDLYFENAMRFFNFSWRVTADQLRKAPNRDQWSMTPPMVNAYYSPTKNEIVFPAGILQAPFYTRSSPKALNFGGIGVVVGHELTHAFDDQGREYDKDGNLRPWWKNSSVEAFKRQTECMVEQYSNYSVNGEPVNGRHTLGENIADNGGLKAAYRAYQNWVKKNGAEHSLPTLGLTNNQLFFLGFAQVWCSVRTPESSHEGLITDPHSPSRFRVIGSLSNSKEFSEHFRCPPGSPMNPPHKCEVW

SEQ ID NO: 29 - DNA, 3346 bp, Homo sapiens
  CDS (790)..(1830)
gagtagacagcacagcggcagcggagggagtctatgcgagctggacagcagtgggaggtttgtgaggctcgcactggccgcagaccctcgggctcgatcgcccgggagccaggactcggcgacgcgaggctgccgggctacccggccgaggcttcgggggcgcaaactaatgggactggctcgctcggcagcatctccccgctcttctaagtacactgagcagggcccgcgctgaagtagaagctgtccgggggcgcgtagcccggagtcccagtgtggcccggaggaacggagcccgtgccagggcggcccagtcgggagcccggggaccgagcttgtgctgtggggaaacccccacttcttccaagggacagcgatcccgggacggtcgaggcgtcggggcggtcaccgagacctctgcgggaagaccccgtcggggagagggcgcgcagccccgaagcgtctcgggaagtcgagcggaatcgggcgggatcacccgggggcgcagagcccccgtcgcgcctcgtgcggcagcggagagcccaggagaacgagccctcgggggccgaagcccatgcccgggttgggggcggctgcccagtgagtcctcctggccggccgggcggagaagagcgacaccgaagccggcgggaggggagcacttcaaggccggcggctgcggaggatgggcgcctgagcggctccgagcgcagcgcggcagaggaaggcgaggcgagctttggtgaggaggcgccaagggatcccgaagtgcagtctgcccccgggaagatggctcggcctgggcagcgttggctcggcaagtggcttgtggcgatggtcgtgtgggcgctgtgccggctcgccacaccgctggccaagaacctggagcccgtatcctggagctccctcaaccccaagttcctgagtgggaagggcttggtgatctatccgaaaattggagacaagctggacatcatctgcccccgagcagaagcagggcggccctatgagtactacaagctgtacctggtgcggcctgagcaggcagctgcctgtagcacagttctcgaccccaacgtgttggtcacctgcaataggccagagcaggaaatacgctttaccatcaagttccaggagttcagccccaactacatgggcctggagttcaagaagcaccatgattactacattacctcaacatccaatggaagcctggaggggctggaaaaccgggagggcggtgtgtgccgcacacgcaccatgaagatcatcatgaaggttgggcaagatcccaatgctgtgacgcctgagcagctgactaccagcaggcccagcaaggaggcagacaacactgtcaagatggccacacaggcccctggtagtcggggctccctgggtgactctgatggcaagcatgagactgtgaaccaggaagagaagagtggcccaggtgcaagtgggggcagcagcggggaccctgatggcttcttcaactccaaggtggcattgttcgcggctgtcggtgccggttgcgtcatcttcctgctcatcatcatcttcctgacggtcctactactgaagctacgcaagcggcaccgcaagcacacacagcagcgggcggctgccctctcgctcagtaccctggccagtcccaaggggggcagtggcacagcgggcaccgagcccagcgacatcatcattcccttacggactacagagaacaactactgcccccactatgagaaggtgagtggggactacgggcaccctgtctacatcgtccaagagatgccgccccagagcccggcgaacatctactacaaggtctgagtgcccggcacggcctcaggcccccgagggacagtcggcctggaccggacctctcctttcgcccccacaccccctccccttgccagctgtgcccacctttgtatttagttttgtagtttcttggcttttataatccccctttttccctgccccctgggcttcggaggggggtgcttgtgcccctaacccccatgctcttgtgccttccccctctggccaggcctctgggctccgtgggggcgccccttcttggaaggcagggctggacactgatggacagcaggcagggagacagtcccctggccctgcccctccctcgccccccttgccaccttcccaggactgcttgtccgctatcatcactgtttttaatgcttttgtgttcattttttagctgtcaactcattttcatctgttttttgaagaaaaatggaaaaatgtaaaaggcagcccctccccaggctttgtgagcctggcccaagccagtacaagagggcctggggcacgatgtggtcagccaggaagcataggatgccatttcttttatagattccttggtatttctggtggggtaaggggcaggccagggctgttcacgcccatgagggaagaggaaagtgccactgggcaaggtgtcccaccctcccctcctgaccctcctacgaggcttatcctggcaatggggtagtcactgccacccttccacacacacacacacacacacacacacaaaaaaaaatcccttccttgtgggattcttgggcatctcctgcctccctcactctcacggtaattaatgtcttaattggctgttgcctggggaacaggagagctgctgcaggcagatgacctcatggggggtggagggaggtgaggtgcccaggtggctatttgccctgcagagctgggagtttcacccccaccccccaccctgttctctccttacctttggcatcctttggcctggtggggaaacagaggcccagggtggagacctaagcgggtataagaccaggtggcctgctccttttctgggccctagcacaggtgggtaacccccacccaacccagctcctgctgctgtcccagtcttgggctggggcctggaaagaggaagaggctgcctggggctgggccagcccgctgtgcactttgaccccagttccttgccagcacggctgctaacagactgccacttgagtgcgccttgcaggcactcccagagcagccatggaaggagctggccctcacaccatccacctccacactgcctcctggccagctgcccaccccagtgccaggtgggagagggagcagaacagccagccccttccaggtggcagtcggaagggtttttgtttttgtttctgttgccatttgtgtaaatactagtctttttggaaaaaaaataatgtaaagatgttttgtataaactctgaattattttcttgttgcttttttcttagaaaaaaatgagaactaaaaaaaaaaaattaaccacatggaaaaaaaaaaa

SEQ ID NO: 30 - protein, 346 aa, Homo sapiens
MARPGQRWLGKWLVAMVVWALCRLATPLAKNLEPVSWSSLNPKFLSGKGLVIYPKIGDKLDIICPRAEAGRPYEYYKLYLVRPEQAAACSTVLDPNVLVTCNRPEQEIRFTIKFQEFSPNYMGLEFKKHHDYYITSTSNGSLEGLENREGGVCRTRTMKIIMKVGQDPNAVTPEQLTTSRPSKEADNTVKMATQAPGSRGSLGDSDGKHETVNQEEKSGPGASGGSSGDPDGFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 31 - DNA, 2488 bp, Homo sapiens
  CDS (127)..(1266)
cggcccaacgaggcgctggtggtttcagggggctgttgtggttccgactataaacagtacgtgtttggcggctgggcctgggcctggtggtgtatctccgacactcagaggatttccctagagattatgacgttgcagccccgctgcgaggacgtagagacggccgagggggtagctttaactgtgacgggtgtcgcccaggtgaagatcatgacggagaaggaactcctggccgtggcttgtgagcagtttctgggtaagaatgtgcaggacatcaaaaacgtcgtcctgcagaccctggagggacatctgcgctccatcctcgggaccctgacagtggagcagatttatcaggaccgggaccagtttgccaagctggtgcgggaggtggcagcccctgatgttggccgcatgggcattgagatcctcagcttcaccatcaaggacgtgtatgacaaagtggactatctgagctccctgggcaagacgcagactgccgtggtgcagagagatgctgacattggcgtggccgaggctgaacgggacgcaggcatccgggaagctgagtgcaagaaggagatgctggatgtgaagttcatggcagacaccaagattgctgactctaagcgagccttcgagctgcaaaagtcagccttcagtgaggaggttaacatcaagacagctgaggcccagttggcctatgagctgcagggggcccgtgaacagcagaagatccggcaggaagagattgagattgaggttgtgcagcgcaagaaacagattgccgtggaggcacaggagatcctgcgtacggacaaggagctcatcgctacagtgcgccggcctgccgaggccgaggcccaccgcatccagcagattgccgagggtgaaaaggtgaagcaggtcctcttggcacaggcagaggctgagaagatccgcaaaatcggggaggcggaagcggcagtcatcgaggcgatgggcaaggcagaggctgagcggatgaagctcaaggcagaagcctaccagaaatacggggatgcagccaagatggccttggtgctagaggccctgccccagattgctgccaaaatcgctgccccacttaccaaggtcgatgagattgtggtcctcagtggagacaacagtaaggtcacatcagaagtgaaccgactgctggccgagctgcctgcctctgtgcatgccctcacaggcgtggacctgtctaagatacccctgatcaagaaggccactggtgtgcaggtgtgaggctcctacaggcccactctcttcagcagccacccggccctccctccagcacccgttttaatcccacagaacaacgggaacgttactgactctggtgccttatctcgaagggaccagaagtgctgcgtgttcaggccatctctggctgtcttcctgtctctcctgtctgtccacctcctcctcttcctctcctttaccccactttcactgccactttcatcaggtttgtgtctcatctccctgcgtgtcttttcctttgtctgtctttttctttcccccatgcacatcatgtagattaagctgaagatgtttattacaatcactctctgtggggggtggccctgctgctcctcagaatcctggtgccttgaagttctctgtgcatctgtccatcctccctatggccctggccagagctcagcatgggcaggggttctgggtaggacggtcactgtcctctctcctggactggtcttcccagccctaaaccctgccccaggaagcccacagcctcacctgctgctgcccctctaggtctgggcagccatgacctgcagggcccagagacactgtccttcccctcatccacccaaggccccagccagcgctcataccctgtcctttctccctgaccccaagggcacagaggcaaggcctcctgtctacagcagcttcctcagtttcctactgccttaggaggcccctgcttgtgctcagggaaggcctcttcatgggcatgttcctgctggggcggtgcggtttggtcccaactctgctaagttttctgagatgagggtctagccctgttggggacagaaaagtgtgtagaccttcttcctgctagggctgcactgtcctgggtgttgggcccttctggtggacaaggctgtgccaaccctgtacagaatcgagtgctgtagcctggccagaccccagagcccttgtgccatctttcttcctggccagagtgatggggttccagccatggggaagcaacccaatcctctgtctccttgctccaatggaggcagaagagcccaggacccaagcgtcttggcaggggtgctgtgaatgtccagtggtcccagctccccaccctggccctgccccagcctgtgtagctcttcctgcatgtggatgctgcatgtctggtctggggcttggatgttgcactgccccactgcctgtcccttctggtaaaataaagaactcttaatgcccg

SEQ ID NO: 32 - protein, 379 aa, Homo sapiens
MTLQPRCEDVETAEGVALTVTGVAQVKIMTEKELLAVACEQFLGKNVQDIKNVVLQTLEGHLRSILGTLTVEQIYQDRDQFAKLVREVAAPDVGRMGIEILSFTIKDVYDKVDYLSSLGKTQTAVVQRDADIGVAEAERDAGIREAECKKEMLDVKFMADTKIADSKRAFELQKSAFSEEVNIKTAEAQLAYELQGAREQQKIRQEEIEIEVVQRKKQIAVEAQEILRTDKELIATVRRPAEAEAHRIQQIAEGEKVKQVLLAQAEAEKIRKIGEAEAAVIEAMGKAEAERMKLKAEAYQKYGDAAKMALVLEALPQIAAKIAAPLTKVDEIVVLSGDNSKVTSEVNRLLAELPASVHALTGVDLSKIPLIKKATGVQV

SEQ ID NO: 33 - DNA, 1771 bp, Homo sapiens
  CDS (7)..(1650)
gtcagaatggccaccatggtaccatccgtgttgtggcccagggcctgctggactctgctggtctgctgtctgctgaccccaggtgtccaggggcaggagttccttttgcgggtggagccccagaaccctgtgctctctgctggagggtccctgtttgtgaactgcagtactgattgtcccagctctgagaaaatcgccttggagacgtccctatcaaaggagctggtggccagtggcatgggctgggcagccttcaatctcagcaacgtgactggcaacagtcggatcctctgctcagtgtactgcaatggctcccagataacaggctcctctaacatcaccgtgtacaggctcccggagcgtgtggagctggcacccctgcctccttggcagccggtgggccagaacttcaccctgcgctgccaagtggaggatgggtcgccccggaccagcctcacggtggtgctgcttcgctgggaggaggagctgagccggcagcccgcagtggaggagccagcggaggtcactgccactgtgctggccagcagagacgaccacggagcccctttctcatgccgcacagaactggacatgcagccccaggggctgggactgttcgtgaacacctcagccccccgccagctccgaacctttgtcctgcccgtgacccccccgcgcctcgtggccccccggttcttggaggtggaaacgtcgtggccggtggactgcaccctagacgggctttttccagcctcagaggcccaggtctacctggcgctgggggaccagatgctgaatgcgacagtcatgaaccacggggacacgctaacggccacagccacagccacggcgcgcgcggatcaggagggtgcccgggagatcgtctgcaacgtgaccctagggggcgagagacgggaggcccgggagaacttgacggtctttagcttcctaggacccattgtgaacctcagcgagcccaccgcccatgaggggtccacagtgaccgtgagttgcatggctggggctcgagtccaggtcacgctggacggagttccggccgcggccccggggcagccagctcaacttcagctaaatgctaccgagagtgacgacggacgcagcttcttctgcagtgccactctcgaggtggacggcgagttcttgcacaggaacagtagcgtccagctgcgagtcctgtatggtcccaaaattgaccgagccacatgcccccagcacttgaaatggaaagataaaacgagacacgtcctgcagtgccaagccaggggcaacccgtaccccgagctgcggtgtttgaaggaaggctccagccgggaggtgccggtggggatcccgttcttcgtcaacgtaacacataatggtacttatcagtgccaagcgtccagctcacgaggcaaatacaccctggtcgtggtgatggacattgaggctgggagctcccactttgtccccgtcttcgtggcggtgttactgaccctgggcgtggtgactatcgtactggccttaatgtacgtcttcagggagcaccaacggagcggcagttaccatgttagggaggagagcacctatctgcccctcacgtctatgcagccgacagaagcaatgggggaagaaccgtccagagctgagtgacgctgggatccgggatcaaagttggcgggggcttggctgtgccctcagattccgcaccaataaagccttcaaactccctaaaaaaaaaaaaaaaaaaaaaaaaaaaaaaaaaaaaaaaaaa

SEQ ID NO: 34 - protein, 547 aa, Homo sapiens
MATMVPSVLWPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQPVGQNFTLRCQVEDGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 35 - DNA, 1314 bp, Homo sapiens
  CDS (128)..(1159)
cggctgtgttgcgcagtcttcatgggttcccgacgaggaggtctctgtggctgcggcggcggctgctaactgcgccacctgctgcagcctgtccccgccgctctgaagcggccgcgtcgaagccgaaatgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatggtttcctcatgaggacaaatacctgattttgaataaagcagcattcagttgaaataaccctttctgtggtaattccaagtgaatatttttcttctaggtgatgagtttctacttcctctggtttttacaacaggaaatgaaatggtatctaaaataaacaagctgtggtatgatgataatt

SEQ ID NO: 36 - protein, 343 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGFLMRTNT

SEQ ID NO: 37 - DNA, 5077 bp, Homo sapiens
  CDS (405)..(4121)
ctcatgcatatgcaggtgcgcgggtgacgaatgggcgagcgagctgtcagtctcgttccgaacttgttggctgcggtgccgggagcgcgggcgcgcagagccgaggccgggacccgctgccttcaccgccgccgccgtcgccgccgggtgggagccgggccgggcagccggagcgcggccgccagcgagccggagctgccgccgcccctgcacgcccgccgcccaggcccgcgcgccgcggcgctgcgctcgaccccgcccgcgccgccgccgccgccgcctctgccgctgccgctgcctctgcgggcgctcggagggcgggcgggcgctgggaggccggcgcggcggctgggagccgggcgcgggcggcggcggcggggccgggcgggcgggtcgcgggggcaatgcgggcgcagggccgggggcgccttccccggcggctgctgctgctgctggcgctctgggtgcaggcggcgcggcccatgggctatttcgagctgcagctgagcgcgctgcggaacgtgaacggggagctgctgagcggcgcctgctgtgacggcgacggccggacaacgcgcgcggggggctgcggccacgacgagtgcgacacgtacgtgcgcgtgtgccttaaggagtaccaggccaaggtgacgcccacggggccctgcagctacggccacggcgccacgcccgtgctgggcggcaactccttctacctgccgccggcgggcgctgcgggggaccgagcgcgggcgcgggcccgggccggcggcgaccaggacccgggcctcgtcgtcatccccttccagttcgcctggccgcgctcctttaccctcatcgtggaggcctgggactgggacaacgataccaccccgaatgaggagctgctgatcgagcgagtgtcgcatgccggcatgatcaacccggaggaccgctggaagagcctgcacttcagcggccacgtggcgcacctggagctgcagatccgcgtgcgctgcgacgagaactactacagcgccacttgcaacaagttctgccggccccgcaacgactttttcggccactacacctgcgaccagtacggcaacaaggcctgcatggacggctggatgggcaaggagtgcaaggaagctgtgtgtaaacaagggtgtaatttgctccacgggggatgcaccgtgcctggggagtgcaggtgcagctacggctggcaagggaggttctgcgatgagtgtgtcccctaccccggctgcgtgcatggcagttgtgtggagccctggcagtgcaactgtgagaccaactggggcggcctgctctgtgacaaagacctgaactactgtggcagccaccacccctgcaccaacggaggcacgtgcatcaacgccgagcctgaccagtaccgctgcacctgccctgacggctactcgggcaggaactgtgagaaggctgagcacgcctgcacctccaacccgtgtgccaacgggggctcttgccatgaggtgccgtccggcttcgaatgccactgcccatcgggctggagcgggcccacctgtgcccttgacatcgatgagtgtgcttcgaacccgtgtgcggccggtggcacctgtgtggaccaggtggacggctttgagtgcatctgccccgagcagtgggtgggggccacctgccagctggacgccaatgagtgtgaagggaagccatgccttaacgctttttcttgcaaaaacctgattggcggctattactgtgattgcatcccgggctggaagggcatcaactgccatatcaacgtcaacgactgtcgcgggcagtgtcagcatgggggcacctgcaaggacctggtgaacgggtaccagtgtgtgtgcccacggggcttcggaggccggcattgcgagctggaacgagacgagtgtgccagcagcccctgccacagcggcggcctctgcgaggacctggccgacggcttccactgccactgcccccagggcttctccgggcctctctgtgaggtggatgtcgacctttgtgagccaagcccctgccggaacggcgctcgctgctataacctggagggtgactattactgcgcctgccctgatgactttggtggcaagaactgctccgtgccccgcgagccgtgccctggcggggcctgcagagtgatcgatggctgcgggtcagacgcggggcctgggatgcctggcacagcagcctccggcgtgtgtggcccccatggacgctgcgtcagccagccagggggcaacttttcctgcatctgtgacagtggctttactggcacctactgccatgagaacattgacgactgcctgggccagccctgccgcaatgggggcacatgcatcgatgaggtggacgccttccgctgcttctgccccagcggctgggagggcgagctctgcgacaccaatcccaacgactgccttcccgatccctgccacagccgcggccgctgctacgacctggtcaatgacttctactgtgcgtgcgacgacggctggaagggcaagacctgccactcacgcgagttccagtgcgatgcctacacctgcagcaacggtggcacctgctacgacagcggcgacaccttccgctgcgcctgcccccccggctggaagggcagcacctgcgccgtcgccaagaacagcagctgcctgcccaacccctgtgtgaatggtggcacctgcgtgggcagcggggcctccttctcctgcatctgccgggacggctgggagggtcgtacttgcactcacaataccaacgactgcaaccctctgccttgctacaatggtggcatctgtgttgacggcgtcaactggttccgctgcgagtgtgcacctggcttcgcggggcctgactgccgcatcaacatcgacgagtgccagtcctcgccctgtgcctacggggccacgtgtgtggatgagatcaacgggtatcgctgtagctgcccacccggccgagccggcccccggtgccaggaagtgatcgggttcgggagatcctgctggtcccggggcactccgttcccacacggaagctcctgggtggaagactgcaacagctgccgctgcctggatggccgccgtgactgcagcaaggtgtggtgcggatggaagccttgtctgctggccggccagcccgaggccctgagcgcccagtgcccactggggcaaaggtgcctggagaaggccccaggccagtgtctgcgaccaccctgtgaggcctggggggagtgcggcgcagaagagccaccgagcaccccctgcctgccacgctccggccacctggacaataactgtgcccgcctcaccttgcatttcaaccgtgaccacgtgccccagggcaccacggtgggcgccatttgctccgggatccgctccctgccagccacaagggctgtggcacgggaccgcctgctggtgttgctttgcgaccgggcgtcctcgggggccagtgccgtggaggtggccgtgtccttcagccctgccagggacctgcctgacagcagcctgatccagggcgcggcccacgccatcgtggccgccatcacccagcgggggaacagctcactgctcctggctgtcaccgaggtcaaggtggagacggttgttacgggcggctcttccacaggtctgctggtgcctgtgctgtgtggtgccttcagcgtgctgtggctggcgtgcgtggtcctgtgcgtgtggtggacacgcaagcgcaggaaagagcgggagaggagccggctgccgcgggaggagagcgccaacaaccagtgggccccgctcaaccccatccgcaaccccatcgagcggccggggggccacaaggacgtgctctaccagtgcaagaacttcacgccgccgccgcgcagggcggacgaggcgctgcccgggccggccggccacgcggccgtcagggaggatgaggaggacgaggatctgggccgcggtgaggaggactccctggaggcggagaagttcctctcacacaaattcaccaaagatcctggccgctcgccggggaggccggcccactgggcctcaggccccaaagtggacaaccgcgcggtcaggagcatcaatgaggcccgctacgccggcaaggagtaggggcggctgccagctgggccgggacccagggccctcggtgggagccatgccgtctgccggacccggaggccgaggccatgtgcatagtttctttattttgtgtaaaaaaaccaccaaaaacaaaaaccaaatgtttattttctacgtttctttaaccttgtataaattattcagtaactgtcaggctgaaaacaatggagtattctcggatagttgctatttttgtaaagtttccgtgcgtggcactcgctgtatgaaaggagagagcaaagggtgtctgcgtcgtcaccaaatcgtagcgtttgttaccagaggttgtgcactgtttacagaatcttccttttattcctcactcgggtttctctgtggctccaggccaaagtgccggtgagacccatggctgtgttggtgtggcccatggctgttggtgggacccgtggctgatggtgtggcctgtggctgtcggtgggactcgtggctgtcaatgggacctgtggctgtcggtgggacctacggtggtcggtgggaccctggttattgatgtggccctggctgccggcacggcccgtggctgttgacgcacctgtggttgttagtggggcctgaggtcatcggcgtggcccaaggccggcaggtcaacctcgcgcttgctggccagtccaccctgcctgccgtctgtgcttcctcctgcccagaacgcccgctccagcgatctctccactgtgctttcagaagtgcccttcctgctgcgcagttctcccatcctgggacggcggcagtattgaagctcgtgacaagtgccttcacacagacccctcgcaactgtccacgcgtgccgtggcaccaggcgctgcccacctgccggccccggccgcccctcctcgtgaaagtgcatttttgtaaatgtgtacatattaaaggaagcactctgtatatttgattgaataatgccacca

SEQ ID NO: 38 - protein, 1238 aa, Homo sapiens
MRAQGRGRLPRRLLLLLALWVQAARPMGYFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCEKAEHACTSNPCANGGSCHEVPSGFECHCPSGWSGPTCALDIDECASNPCAAGGTCVDQVDGFECICPEQWVGATCQLDANECEGKPCLNAFSCKNLIGGYYCDCIPGWKGINCHINVNDCRGQCQHGGTCKDLVNGYQCVCPRGFGGRHCELERDECASSPCHSGGLCEDLADGFHCHCPQGFSGPLCEVDVDLCEPSPCRNGARCYNLEGDYYCACPDDFGGKNCSVPREPCPGGACRVIDGCGSDAGPGMPGTAASGVCGPHGRCVSQPGGNFSCICDSGFTGTYCHENIDDCLGQPCRNGGTCIDEVDAFRCFCPSGWEGELCDTNPNDCLPDPCHSRGRCYDLVNDFYCACDDGWKGKTCHSREFQCDAYTCSNGGTCYDSGDTFRCACPPGWKGSTCAVAKNSSCLPNPCVNGGTCVGSGASFSCICRDGWEGRTCTHNTNDCNPLPCYNGGICVDGVNWFRCECAPGFAGPDCRINIDECQSSPCAYGATCVDEINGYRCSCPPGRAGPRCQEVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTGLLVPVLCGAFSVLWLACVVLCVWWTRKRRKERERSRLPREESANNQWAPLNPIRNPIERPGGHKDVLYQCKNFTPPPRRADEALPGPAGHAAVREDEEDEDLGRGEEDSLEAEKFLSHKFTKDPGRSPGRPAHWASGPKVDNRAVRSINEARYAGKE

SEQ ID NO: 39 - DNA, 634 bp, Homo sapiens
  CDS (3)..(371)
ccagggccgcactccggagactcgcggttgctacgcgcaccatggctggagcgcccacggtctcgcttcctgaactccgttcactcctagcctccggacgggcccggctcttcgacgtgcgctctcgcgaggaggcggcagctgggaccatcccaggggcgctcaacatcccggtgtccgagttggagagtgctctgcagatggagccagctgccttccaggctttatattctgctgagaagccaaagctggaagatgagcatctcgttttcttctgtcagatgggcaagcggggcctccaggccacgcagctggcccggagtcttggatacactgggtacggggaggtgtggctgctagctgggaggtgatggggactgcctgtcattcctgtcagtctctcacgcttctttgtctccacagggctcgcaactacgctggagcctatagagaatggttggagaaagagagttaggcaggaggcagcttactgattgccaccccctggccccttaatggccaccttaactaagggtgtgaacgggctgacttggtgaattgggcaactccttatagtgttgtgcacacaaaagcatcaaataaagaacatttaatcaaaaaaaaaaaaaaaaaa

SEQ ID NO: 40 - protein, 122 aa, Homo sapiens
RAALRRLAVATRTMAGAPTVSLPELRSLLASGRARLFDVRSREEAAAGTIPGALNIPVSELESALQMEPAAFQALYSAEKPKLEDEHLVFFCQMGKRGLQATQLARSLGYTGYGEVWLLAGR

SEQ ID NO: 41 - DNA, 2254 bp, Homo sapiens
  CDS (180)..(1937)
aatcgaaagtagactcttttctgaagcatttcctgggatcagcctgaccacgctccatactgggagaggcttctgggtcaaaggaccagtctgcagagggatcctgtggctggaagcgaggaggctccacacggccgttgcagctaccgcagccaggatctgggcatccaggcacggccatgacccctccgaggctcttctgggtgtggctgctggttgcaggaacccaaggcgtgaacgatggtgacatgcggctggccgatgggggcgccaccaaccagggccgcgtggagatcttctacagaggccagtggggcactgtgtgtgacaacctgtgggacctgactgatgccagcgtcgtctgccgggccctgggcttcgagaacgccacccaggctctgggcagagctgccttcgggcaaggatcaggccccatcatgctggacgaggtccagtgcacgggaaccgaggcctcactggccgactgcaagtccctgggctggctgaagagcaactgcaggcacgagagagacgctggtgtggtctgcaccaatgaaaccaggagcacccacaccctggacctctccagggagctctcggaggcccttggccagatctttgacagccagcggggctgcgacctgtccatcagcgtgaatgtgcagggcgaggacgccctgggcttctgtggccacacggtcatcctgactgccaacctggaggcccaggccctgtggaaggagccgggcagcaatgtcaccatgagtgtggatgctgagtgtgtgcccatggtcagggaccttctcaggtacttctactcccgaaggattgacatcaccctgtcgtcagtcaagtgcttccacaagctggcctctgcctatggggccaggcagctgcagggctactgcgcaagcctctttgccatcctcctcccccaggacccctcgttccagatgcccctggacctgtatgcctatgcagtggccacaggggacgccctgctggagaagctctgcctacagttcctggcctggaacttcgaggccttgacgcaggccgaggcctggcccagtgtccccacagacctgctccaactgctgctgcccaggagcgacctggcggtgcccagcgagctggccctactgaaggccgtggacacctggagctggggggagcgtgcctcccatgaggaggtggagggcttggtggagaagatccgcttccccatgatgctccctgaggagctctttgagctgcagttcaacctgtccctgtactggagccacgaggccctgttccagaagaagactctgcaggccctggaattccacactgtgcccttccagttgctggcccggtacaaaggcctgaacctcaccgaggatacctacaagccccggatttacacctcgcccacctggagtgcctttgtgacagacagttcctggagtgcacggaagtcacaactggtctatcagtccagacgggggcctttggtcaaatattcttctgattacttccaagccccctctgactacagatactacccctaccagtccttccagactccacaacaccccagcttcctcttccaggacaagagggtgtcctggtccctggtctacctccccaccatccagagctgctggaactacggcttctcctgctcctcggacgagctccctgtcctgggcctcaccaagtctggcggctcagatcgcaccattgcctacgaaaacaaagccctgatgctctgcgaagggctcttcgtggcagacgtcaccgatttcgagggctggaaggctgcgattcccagtgccctggacaccaacagctcgaagagcacctcctccttcccctgcccggcagggcacttcaacggcttccgcacggtcatccgccccttctacctgaccaactcctcaggtgtggactagacgcgtggccaagggtggtgagaaccggagaaccccaggacgccctcactgcaggctcccctcctcggcttccttcctctctgcaatgaccttcaacaaccggccaccagatgtcgccctactcacctgaggctcagcttcaagaaattactggaaggcttccactagggtccaccaggagttctcccaccacctcaccagtttccaggtggtaagcaccaggaggccctcgaggttgctctggatccccccacagcccctggtcagtctgcccttgtcactggtctgaggtcattaaaattacattgaggttccta

SEQ ID NO: 42 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 43 - DNA, 1185 bp, Homo sapiens
  CDS (283)..(1131)
gggctcttctggtatttctcggcctgcgagaagtgtgtgctggcccaggtgtgcaaggcctggcggcgcgtgctgtaccagcccaagttctgggcaggcctcacgccggtgctgcatgccaaggagctctacaacgtgctgcctggtggcgagaaggagttcgtgaacctgcagggttttgccgccagaggcttcgagggcttctgcctggttggcgtctccgacctggacatctgtgagttcattgacaactatgcgctctccaagaagggtgtcaaagccatgagcctcaagcgctccaccatcacggacgcaggcctcgaggttatgcttgaacagatgcagggcgtggtgcgtctggagctgtcgggctgcaacgacttcaccgaggccgggctgtggtccagcctgagcgcgcgcatcacctcgctgagcgtgagtgactgcatcaacgtggccgacgacgccatcgcggccatctcgcagctgctgcccaacctggcggagctgagcctgcaggcctaccacgtgacggacacggcgctggcctacttcacggcgcgccagggccacagcacgcacacgctgcgcctgctctcctgctgggagatcaccaaccacggcgtggtcaacgtggtgcacagcctgcccaacctcaccgcgctcagcctctcgggctgctccaaggtcaccgacgacggcgtggagctcgtggccgagaacctgcgcaagctgcgcagccttgacctctcgtggtgcccacgcatcaccgacatggcgctggagtacgtggcctgcgacctgcaccgcctagaggagctcgtgctcgacaggtgtgtacgcatcacggacactggcctcagctatctgtccaccatgtcgtccctccgcagcctctacctgcgatggtgctgccaggtgcaagacttcgggctgaagcacctcctggccctggggagtttgcgcctcctgtctctggcaggctgcccgctgctcaccaccaccgggctgtcgggcctggtgcagctgcaggagctggaggagctggagctgaccaactgccccggggccacccccgagctcttcaagtatttctcgcagcacctgccccgctgcctcgtcattgagtagcgcgaggcccccgccccggtcgcgggaacccggccatgacctgggcgggggcgc

SEQ ID NO: 44 - protein, 282 aa, Homo sapiens
MSLKRSTITDAGLEVMLEQMQGVVRLELSGCNDFTEAGLWSSLSARITSLSVSDCINVADDAIAAISQLLPNLAELSLQAYHVTDTALAYFTARQGHSTHTLRLLSCWEITNHGVVNVVHSLPNLTALSLSGCSKVTDDGVELVAENLRKLRSLDLSWCPRITDMALEYVACDLHRLEELVLDRCVRITDTGLSYLSTMSSLRSLYLRWCCQVQDFGLKHLLALGSLRLLSLAGCPLLTTTGLSGLVQLQELEELELTNCPGATPELFKYFSQHLPRCLVIE

SEQ ID NO: 45 - DNA, 1780 bp, Homo sapiens
  CDS (190)..(987)
aggaagtggcgcggaccttcatttggggtttcggttcccccccttccccttccccggggtctgggggtgacattgcaccgcgcccctcgtggggtcgcgttgccaccccacgcggactccccagctggcgcgcccctcccatttgcctgtcctggtcaggcccccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagcctcttgtgccgacggcaggaggacagtcgggtgatggtgtattctgccctgcgcatcccacccgaggactgagggaacctaggggggacccctgggcctggggtgccctcctgatgtcctcgccctgtatttctccatctccagttctggacagtgcaggttgccaagaaaagggacctagtttagccattgccctggagatgaaattaatggaggctcaaggatagatgagctctgagtttctcagtactccctcaagactggacatcttggtctttttctcaggcctgagggggaaccatttttggtgtgataaataccctaaactgcctttttttcttttttgaggtggggggagggaggaggtatattggaactcttctaacctccttgggctatattttctctcctcgagttgctcctcatggctgggctcatttcggtccctttctccttggtcccagaccttgggggaaaggaaggaagtgcatgtttgggaactggcattactggaactaatggttttaacctccttaaccaccagcatccctcctctccccaaggtgaagtggagggtgctgtggtgagctggccactccagagctgcagtgccactggaggagtcagactaccatgacatcgtagggaaggaggggagatttttttgtagtttttaattggggtgtgggaggggcggggaggttttctataaactgtatcattttctgctgagggtggagtgtcccatccttttaatcaaggtgattgtgattttgactaataaaaaagaatttgtaaaaaaaaaaaaaaaaaaaaaaaaaaaaaaaaaaaaaaaaaaaaaaaaaaaaaaaaaaaaaaaaaaaaa

SEQ ID NO: 46 - protein, 265 aa, Homo sapiens
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSLLCRRQEDSRVMVYSALRIPPED

SEQ ID NO: 47 - DNA, 3549 bp, Homo sapiens
  CDS (175)..(1602)
cccctcttcctcctcctcaagggaaagctgcccacttctagctgccctgccatcccctttaaagggcgacttgctcagcgccaaaccgcggctccagccctctccagcctccggctcagccggctcatcagtcggtccgcgccttgcagctcctccagagggacgcgccccgagatggagagcaaagccctgctcgtgctgactctggccgtgtggctccagagtctgaccgcctcccgcggaggggtggccgccgccgaccaaagaagagattttatcgacatcgaaagtaaatttgccctaaggacccctgaagacacagctgaggacacttgccacctcattcccggagtagcagagtccgtggctacctgtcatttcaatcacagcagcaaaaccttcatggtgatccatggctggacggtaacaggaatgtatgagagttgggtgccaaaacttgtggccgccctgtacaagagagaaccagactccaatgtcattgtggtggactggctgtcacgggctcaggagcattacccagtgtccgcgggctacaccaaactggtgggacaggatgtggcccggtttatcaactggatggaggaggagtttaactaccctctggacaatgtccatctcttgggatacagccttggagcccatgctgctggcattgcaggaagtctgaccaataagaaagtcaacagaattactggcctcgatccagctggacctaactttgagtatgcagaagccccgagtcgtctttctcctgatgatgcagattttgtagacgtcttacacacattcaccagagggtcccctggtcgaagcattggaatccagaaaccagttgggcatgttgacatttacccgaatggaggtacttttcagccaggatgtaacattggagaagctatccgcgtgattgcagagagaggacttggagatgtggaccagctagtgaagtgctcccacgagcgctccattcatctcttcatcgactctctgttgaatgaagaaaatccaagtaaggcctacaggtgcagttccaaggaagcctttgagaaagggctctgcttgagttgtagaaagaaccgctgcaacaatctgggctatgagatcaataaagtcagagccaaaagaagcagcaaaatgtacctgaagactcgttctcagatgccctacaaagtcttccattaccaagtaaagattcatttttctgggactgagagtgaaacccataccaatcaggcctttgagatttctctgtatggcaccgtggccgagagtgagaacatcccattcactctgcctgaagtttccacaaataagacctactccttcctaatttacacagaggtagatattggagaactactcatgttgaagctcaaatggaagagtgattcatactttagctggtcagactggtggagcagtcccggcttcgccattcagaagatcagagtaaaagcaggagagactcagaaaaaggtgatcttctgttctagggagaaagtgtctcatttgcagaaaggaaaggcacctgcggtatttgtgaaatgccatgacaagtctctgaataagaagtcaggctgaaactgggcgaatctacagaacaaagaacggcatgtgaattctgtgaagaatgaagtggaggaagtaacttttacaaaacatacccagtgtttggggtgtttcaaaagtggattttcctgaatattaatcccagccctacccttgttagttattttaggagacagtctcaagcactaaaaagtggctaattcaatttatggggtatagtggccaaatagcacatcctccaacgttaaaagacagtggatcatgaaaagtgctgttttgtcctttgagaaagaaataattgtttgagcgcagagtaaaataaggctccttcatgtggcgtattgggccatagcctataattggttagaacctcctattttaattggaattctggatctttcggactgaggccttctcaaactttactctaagtctccaagaatacagaaaatgcttttccgcggcacgaatcagactcatctacacagcagtatgaatgatgttttagaatgattccctcttgctattggaatgtggtccagacgtcaaccaggaacatgtaacttggagagggacgaagaaagggtctgataaacacagaggttttaaacagtccctaccattggcctgcatcatgacaaagttacaaattcaaggagatataaaatctagatcaattaattcttaataggctttatcgtttattgcttaatccctctctcccccttcttttttgtctcaagattatattataataatgttctctgggtaggtgttgaaaatgagcctgtaatcctcagctgacacataatttgaatggtgcagaaaaaaaaaagataccgtaattttattattagattctccaaatgattttcatcaatttaaaatcattcaatatctgacagttactcttcagttttaggcttaccttggtcatgcttcagttgtacttccagtgcgtctcttttgttcctggctttgacatgaaaagataggtttgagttcaaattttgcattgtgtgagcttctacagattttagacaaggaccgtttttactaagtaaaagggtggagaggttcctggggtggattcctaagcagtgcttgtaaaccatcgcgtgcaatgagccagatggagtaccatgagggttgttatttgttgtttttaacaactaatcaagagtgagtgaacaactatttataaactagatctcctatttttcagaatgctcttctacgtataaatatgaaatgataaagatgtcaaatatctcagaggctatagctgggaacccgactgtgaaagtatgtgatatctgaacacatactagaaagctctgcatgtgtgttgtccttcagcataattcggaagggaaaacagtcgatcaagggatgtattggaacatgtcggagtagaaattgttcctgatgtgccagaacttcgaccctttctctgagagagatgatcgtgcctataaatagtaggaccaatgttgtgattaacatcatcaggcttggaatgaattctctctaaaaataaaatgatgtatgatttgttgttggcatcccctttattaattcattaaatttctggatttgggttgtgacccagggtgcattaacttaaaagattcactaaagcagcacatagcactgggaactctggctccgaaaaactttgttatatatatcaaggatgttctggctttacattttatttattagctgtaaatacatgtgtggatgtgtaaatggagcttgtacatattggaaaggtcattgtggctatctgcatttataaatgtgtggtgctaactgtatgtgtctttatcagtgatggtctcacagagccaactcactcttatgaaatgggctttaacaaaacaagaaagaaacgtacttaactgtgtgaagaaatggaatcagcttttaataaaattgacaacattttattaccac

SEQ ID NO: 48 - protein, 475 aa, Homo sapiens
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 49 - DNA, 5100 bp, Homo sapiens
  CDS (49)..(4896)
atggagcccgagtgagcgcggcgcgggcccgtccggccgccggacaacatggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagccgagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggactttaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacattttcgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatctactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaacagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtagacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgtgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctgacctcggccgggccactctggcttctctgtgcccctgtaaatagttttaaatatgaacaaagaaaaaaatatattttatgatttaaaaaataaatataattgggattttaaaaacatgagaaatgtgaactgtgatggggtgggcagggctgggagaactttgtacagtggaacaaatatttataaacttaattttgtaaaacag

SEQ ID NO: 50 - protein, 1615 aa, Homo sapiens
MEAAPPGPPWPLLLLLLLLLALCGCPAPAAASPLLLFANRRDVRLVDAGGVKLESTIVVSGLEDAAAVDFQFSKGAVYWTDVSEEAIKQTYLNQTGAAVQNVVISGLVSPDGLACDWVGKKLYWTDSETNRIEVANLNGTSRKVLFWQDLDQPRAIALDPAHGYMYWTDWGETPRIERAGMDGSTRKIIVDSDIYWPNGLTIDLEEQKLYWADAKLSFIHRANLDGSFRQKVVEGSLTHPFALTLSGDTLYWTDWQTRSIHACNKRTGGKRKEILSALYSPMDIQVLSQERQPFFHTRCEEDNGGCSHLCLLSPSEPFYTCACPTGVQLQDNGRTCKAGAEEVLLLARRTDLRRISLDTPDFTDIVLQVDDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIALHPVMGLMYWTDWGENPKIECANLDGQERRVLVNASLGWPNGLALDLQEGKLYWGDAKTDKIEVINVDGTKRRTLLEDKLPHIFGFTLLGDFIYWTDWQRRSIERVHKVKASRDVIIDQLPDLMGLKAVNVAKVVGTNPCADRNGGCSHLCFFTPHATRCGCPIGLELLSDMKTCIVPEAFLVFTSRAAIHRISLETNNNDVAIPLTGVKEASALDFDVSNNHIYWTDVSLKTISRAFMNGSSVEHVVEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERVVIADDLPHPFGLTQYSDYIYWTDWNLHSIERADKTSGRNRTLIQGHLDFVMDILVFHSSRQDGLNDCMHNNGQCGQLCLAIPGGHRCGCASHYTLDPSSRNCSPPTTFLLFSQKSAISRMIPDDQHSPDLILPLHGLRNVKAIDYDPLDKFIYWVDGRQNIKRAKDDGTQPFVLTSLSQGQNPDRQPHDLSIDIYSRTLFWTCEATNTINVHRLSGEAMGVVLRGDRDKPRAIVVNAERGYLYFTNMQDRAAKIERAALDGTEREVLFTTGLIRPVALVVDNTLGKLFWVDADLKRIESCDLSGANRLTLEDANIVQPLGLTILGKHLYWIDRQQQMIERVEKTTGDKRTRIQGRVAHLTGIHAVEEVSLEEFSAHPCARDNGGCSHICIAKGDGTPRCSCPVHLVLLQNLLTCGEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATVRPYRPYIIRGMAPPTTPCSTDVCDSDYSASRWKASKYYLDLNSDSDPYPPPPTPHSQYLSAEDSCPPSPATERSYFHLFPPPPSPCTDSS

SEQ ID NO: 51 - DNA, 2479 bp, Homo sapiens
  CDS (6)..(1892)
tgaacatggagcccccggacgcaccggcccaggcgcgcggggccccgcggctgctgttgctcgcagtcctgctggcggcgcacccagatgcccaggcggaggtgcgcttgtctgtacccccgctggtggaggtgatgcgaggaaagtctgtcattctggactgcacccctacgggaacccacgaccattatatgctggaatggttccttaccgaccgctcgggagctcgcccccgcctagcctcggctgagatgcagggctctgagctccaggtcacaatgcacgacacccggggccgcagtcccccataccagctggactcccaggggcgcctggtgctggctgaggcccaggtgggcgacgagcgagactacgtgtgcgtggtgagggcaggggcggcaggcactgctgaggccactgcgcggctcaacgtgtttgcaaagccagaggccactgaggtctcccccaacaaagggacactgtctgtgatggaggactctgcccaggagatcgccacctgcaacagccggaacgggaacccggcccccaagatcacgtggtatcgcaacgggcagcgcctggaggtgcccgtagagatgaacccagagggctacatgaccagccgcacggtccgggaggcctcgggcctgctctccctcaccagcaccctctacctgcggctccgcaaggatgaccgagacgccagcttccactgcgccgcccactacagcctgcccgagggccgccacggccgcctggacagccccaccttccacctcaccctgcactatcccacggagcacgtgcagttctgggtgggcagcccgtccaccccagcaggctgggtacgcgagggtgacactgtccagctgctctgccggggggacggcagccccagcccggagtatacgcttttccgccttcaggatgagcaggaggaagtgctgaatgtgaatctcgaggggaacttgaccctggagggagtgacccggggccagagcgggacctatggctgcagagtggaggattacgacgcggcagatgacgtgcagctctccaagacgctggagctgcgcgtggcctatctggaccccctggagctcagcgaggggaaggtgctttccttacctctaaacagcagtgcagtcgtgaactgctccgtgcacggcctgcccacccctgccctacgctggaccaaggactccactcccctgggcgatggccccatgctgtcgctcagttctatcaccttcgattccaatggcacctacgtatgtgaggcctccctgcccacagtcccggtcctcagccgcacccagaacttcacgctgctggtccaaggctcgccagagctaaagacagcggaaatagagcccaaggcagatggcagctggagggaaggagacgaagtcacactcatctgctctgcccgcggccatccagaccccaaactcagctggagccaattggggggcagccccgcagagccaatccccggacggcagggttgggtgagcagctctctgaccctgaaagtgaccagcgccctgagccgcgatggcatctcctgtgaagcctccaacccccacgggaacaagcgccatgtcttccacttcggcaccgtgagcccccagacctcccaggctggagtggccgtcatggccgtggccgtcagcgtgggcctcctgctcctcgtcgttgctgtcttctactgcgtgagacgcaaagggggcccctgctgccgccagcggcgggagaagggggctccgccgccaggggagccagggctgagccactcggggtcggagcaaccagagcagaccggccttctcatgggaggtgcctccggaggagccaggggtggcagcgggggcttcggagacgagtgctgagccaagaacctcctagaggctgtccctggacctggagctgcaggcatcagagaaccagccctgctcacgccatgcccgcccccgccttccctcttccctcttccctctccctgcccagccctcccttccttcctctgccggcaaggcagggacccacagtggctgcctgcctccgggagggaaggagagggagggtgggtgggtgggagggggccttcctccagggaatgtgactctcccaggccccagaatagctcctggacccaagcccaaggcccagcctgggacaaggctccgagggtcggctggccggagctatttttacctcccgcctcccttgctggtccccccacctgacgtcttgctgcagagtctgacactggattcccccccctcaccccgcccctggtcccactcctgcccccgccctacctccgccccaccccatcatctgtggacactggagtctggaataaatgctgtttgtcacatcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - protein, 628 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 53 - DNA, 3565 bp, Homo sapiens
  CDS (249)..(2378)
agcggagcccgagcggccgcgaaggcccccgctctccgcttctcccgcccctcgcgacccagaggrctgctggctggctaagtccctcccgctcccggctctcgcctcactaggagcggctctcggtgcagcgggacagggcgaagcggcctgcgcccacggagcgcgcgacactgcccggaagggaccgccacccttgccccctcagctgcccactcgtgatttccagcggcctccgcgcgcgcacgatgccctcggccaccagccacagcgggagcggcagcaagtcgtccggaccgccaccgccgtcgggttcctccgggagtgaggcggccgcgggagccggggccgccgcgccggcttctcagcaccccgcaaccggcaccggcgctgtccagaccgaggccatgaagcagattctcggggtgatcgacaagaaacttcggaacctggagaagaaaaagggtaagcttgatgattaccaggaacgaatgaacaaaggggaaaggcttaatcaagatcagctggatgccgtttctaagtaccaggaagtcacaaataatttggagtttgcaaaagaattacagaggagtttcatggcactaagtcaagatattcagaaaacaataaagaagacagcacgtcgggagcagcttatgagagaagaagctgaacagaaacgtttaaaaactgtacttgagctacagtatgttttggacaaattgggagatgatgaagtgcggactgacctgaaacaaggtttgaatggagtgccaatattgtccgaagaggagttgtcattgttggatgaattctataagctagtagaccctgaacgggacatgagcttgaggttgaatgaacagtatgaacatgcctccattcacctgtgggacctgctggaagggaaggaaaaacctgtatgtggaaccacctataaagttctaaaggaaattgttgagcgtgtttttcagtcaaactactttgacagcacccacaaccaccagaatgggctgtgtgaggaagaagaggcagcctcagcacctgcagttgaagaccaggtacctgaagctgaacctgagccagcagaagagtacactgagcaaagtgaagttgaatcaacagagtatgtaaatagacagttcatggcagaaacacagttcaccagtggtgaaaaggagcaggtagatgagtggacagttgaaacggttgaggtggtaaattcactccagcagcaacctcaggctgcatccccttcagtaccagagccccactctttgactccagtggctcaggcagatccccttgtgagaagacagcgagtacaagaccttatggcacaaatgcagggtccctataatttcatacaggattcaatgctggattttgaaaatcagacacttgatcctgccattgtatctgcacagcctatgaatccaacacaaaacatggacatgccccagctggtttgccctccagttcattctgaatctagacttgctcagcctaatcaagttcctgtacaaccagaagcgacacaggttcctttggtatcatccacaagtgaggggtacacagcatctcaacccttgtaccagccttctcatgctacagagcaacgaccacagaaggaaccaattgatcagattcaggcaacaatctctttaaatacagaccagactacagcatcatcatcccttcctgctgcgtctcagcctcaagtatttcaggctgggacaagcaaacctttacatagcagtggaatcaatgtaaatgcagctccattccaatccatgcaaacggtgttcaatatgaatgccccagttcctcctgttaatgaaccagaaactttaaaacagcaaaatcagtaccaggccagttataaccagagcttttctagtcagcctcaccaagtagaacaaacagagcttcagcaagaacagcttcaaacagtggttggcacttaccatggttccccagaccagtcccatcaagtgactggtaaccaccagcagcctcctcagcagaacactggatttccacgtagcaatcagccctattacaatagtcgtggtgtgtctcgtggaggctcccgtggtgctagaggcttgatgaatggataccggggccctgccaatggattcagaggaggatatgatggttaccgcccttcattctctaacactccaaacagtggttatacacagtctcagttcagtgctccccgggattactctggctatcaacgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaatcgccaaaaacacactggccagtgtaccataatatgttaccagaagagttattatctatttgttctccctttcaggaaacttattgtaaagggactgttttcatcccataaagacaggactacaattgtcagctttctattacctggatatggaaggaaactatttttactctgcatgttctgtcctaagcgtcatcttgagccttgcacatgatactcagattcctcacccttgcttaggagtaaaacaatatactttacagggtgataataatctccatagttatttgaagtggcttgaaaaaggcaagattgacttttatgacattggataaaatctacaaatcagccctcgagttattcaatgataactgacaaactaaattatttccctagaaaggaagatgaaaggagtggagtgtggtttggcagaacaactgcatttcacagcttttccagttaaattggagcactgaacgttcagatgcataccaaattatgcatgggtcctaatcacacatataaggctggctaccagctttgacacagcactgttcatctggccaaacaactgtggttaaaaacacatgtaaaatgctttttaacagctgatactgtataagacaaagccaagatgcaaaattaggctttgattggcactttttgaaaaatatgcaacaaatatgggatgtaatccggatggccgcttctgtacttaatgtgaaatatttagatacctttttgaacacttaacagtttctttgagacaatgacttttgtaaggattggtactatctatcattccttatgacatgtacattgtctgtcactaatccttggattttgctgtattgtcacctaaattggtacaggtactgatgaaaatctctagtggataatcataacactctcggtcacatgtttttccttcagcttgaaagcttttttttaaaaggaaaagataccaaatgcctgctgctaccacccttttcaattgctatcttttgaaaggcaccagtatgtgttttagattgatttccctgtttcagggaaatcacggacagtagtttcagttctgatggtataagcaaaacaaataaaacgtttataaaagttgtatcttgaaacactggtgttcaacagctagcagcttatgtgattca

SEQ ID NO: 54 - protein, 709 aa, Homo sapiens
MPSATSHSGSGSKSSGPPPPSGSSGSEAAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLVDPERDMSLRLNEQYEHASIHLWDLLEGKEKPVCGTTYKVLKEIVERVFQSNYFDSTHNHQNGLCEEEEAASAPAVEDQVPEAEPEPAEEYTEQSEVESTEYVNRQFMAETQFTSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTELQQEQLQTVVGTYHGSPDQSHQVTGNHQQPPQQNTGFPRSNQPYYNSRGVSRGGSRGARGLMNGYRGPANGFRGGYDGYRPSFSNTPNSGYTQSQFSAPRDYSGYQRDGYQQNFKRGSGQSGPRGAPRGRGGPPRPNRGMPQMNTQQVN

SEQ ID NO: 55 - DNA, 2131 bp, Homo sapiens
  CDS (374)..(1528)
cgactctcccgggctgccagccgggacgcgcggccgccgccgctgcagacgacgagtccgccctcgtcccgcgcccccggggctcgcggagccaggtctccacctctgggcaggagagttgccgaccacctcgggggtgctttctctgcgcttgaacatctatagctgcttctgaggggctgggagccgggcccctgggagagacgagccatgaaccccccacagcctctgcatttggggacctcaccttaggagagtgccatttacagcttccgccagggcaaaggagctgagcagccatcccaagcccagcccacctccctcccccggcccctggtaggcatggactagcagctgtgagcagccagagctgatgcccggcccccaggggggcagaggcgccgccaccatgagcctgggcaagctctcgcctgtgggctgggtgtccagttcacagggaaagaggcggctgactgcagacatgatcagccacccactcggggacttccgccacaccatgcatgtgggccgtggcggggatgtcttcggggacacgtccttcctcagcaaccacggtggcagctccgggagcacccatcgctcaccccgcagcttcctggccaagaagctgcagctggtgcggagggtgggggcgcccccccggaggatggcatctccccctgcaccctccccggctccaccggccatctcccccatcatcaagaacgccatctccctgccccagctcaaccaggccgcctacgacagcctcgtggttggcaagctcagcttcgacagcagccccaccagctccacggacggccactccagctacggcctggactctgggttctgcaccatctcccgcctgccccgctcggaaaagccgcatgaccgagaccgggatggttccttcccctctgagcccgggcttcgccgctctgactctctcttgtccttccgcctggacctcgaccttgggccctcactcctcagcgagctgctaggggtcatgagcctcccagaagcccctgcagctgagactccagcccccgctgcaaaccccccagcccctactgcaaaccccacgggtcctgctgcaaaccccccagcgcctgctgcaaacccctcagcacctgccgcaacccccacgggtcctgctgcaaatcccccagcccctgccgcaagctccacaccccatggacactgtcccaatggggtaacagctgggttgggcccagtggctgaggtgaagtccagcccagtgggagggggtccccgaggacctgctggccctgccctcggcaggcactggggagcaggctgggatggcggccaccactacccagagatggatgcgcggcaggagcgggtggaggtgctgccccaagcccgggcctcctgggagagcctggacgaagagtggagggcgccccaggcaggcagcaggaccccagtgcccagcacagtgcaagcaaacacctttgaatttgcggatgctgaggaggatgatgaggtcaaggtgtgaggggctggggcacggtcccagggccccacctaggtgcagagccggcccctcacctaacagctggttcctaccagaccggagaggggagaagtcatgttgcccctaaacccctccccacctctgcaggacagacatgggagggaggacagggaaggccaggcttgctctgggacttttatgctcccagaggccctgccaaactgaccacctcccccgactgccactctggacctaatagctgttccttaggccccactccatgccacccccaccagctggaggacccagcctcacagtgtgtcctttgtgccagaccaagcggcccgtggggggtggggggcagggagtgtaccacacagggccattgtctcacctcccaaagggaccgcctgcccccagctcatcccagagcgtccctgctgcaaccctgacagccgtctcccaggccgcttccccaacatccccgccccagcctccctcttaccccagaaaggtcaggtatgacctcccggggaggaatcccacctgcctgtataccccagacttgcctctggggcctgattaaataaggctgttttgataaaaaaaaaaaaaaaaaaaaaa

SEQ ID NO: 56 - protein, 384 aa, Homo sapiens
MPGPQGGRGAATMSLGKLSPVGWVSSSQGKRRLTADMISHPLGDFRHTMHVGRGGDVFGDTSFLSNHGGSSGSTHRSPRSFLAKKLQLVRRVGAPPRRMASPPAPSPAPPAISPIIKNAISLPQLNQAAYDSLVVGKLSFDSSPTSSTDGHSSYGLDSGFCTISRLPRSEKPHDRDRDGSFPSEPGLRRSDSLLSFRLDLDLGPSLLSELLGVMSLPEAPAAETPAPAANPPAPTANPTGPAANPPAPAANPSAPAATPTGPAANPPAPAASSTPHGHCPNGVTAGLGPVAEVKSSPVGGGPRGPAGPALGRHWGAGWDGGHHYPEMDARQERVEVLPQARASWESLDEEWRAPQAGSRTPVPSTVQANTFEFADAEEDDEVKV

SEQ ID NO: 57 - DNA, 4471 bp, Homo sapiens
  CDS (247)..(4230)
gcggaacgctagcggtgttggcgcggagtggaccccggctgcggcccctggcaatggcgccaccatcggtcccggagtcccagtgatgctctgtgccatagagcccccataacttcactactacgtgatagtaaatccccggcaaaaaccagcagcgccttgcaagcccacgccaccccaagcatcccaggactcttctgaaacgactccgggctaccagatcggccgtccagctggaatcaaccgatggaggctccgctgcaaactggaatggtgcttggcgtgatgatcggggccggagtggcggtggtggtcacggccgtgctcatcctcctggtggtgcggaggctgcgagtgccaaaaaccccagccccggatggcccccggtatcggttccggaagagggacaaagtgctcttctatggccggaagattatgcggaaggtgtcacaatccacctcctccctcgtggatacctctgtctccgccacctcccggccacgcatgaggaagaaactgaagatgctcaacattgccaagaagatcctgcgcatccagaaagagacgcccacgctgcagcggaaggagcccccgcccgcagtgctagaagctgacctgaccgagggcgacctggctaactcccatctgccctctgaagtgctttatatgctcaagaacgtccgggtgctgggccacttcgagaagccactcttcctggagctctgccgccacatggtcttccagcggctgggccagggtgactacgtcttccggccgggccagccagatgccagcatctacgtggtgcaggacgggctgctggagctctgtctgccagggcctgacgggaaggagtgtgtggtgaaggaagtggttcctggggacagcgtcaacagccttctcagcatcctggatgtcatcaccggtcaccagcatccccagcggaccgtgtctgcccgggcggcccgggactccacggtgctgcgcctgccggtggaagcattctccgcggtcttcaccaagtacccggagagcttggtgcgggtcgtgcagatcatcatggtgcggctgcagcgagtcaccttcctggcactgcacaactacctgggtctgaccaatgagctcttcagccacgagatccagcccctgcgtctgttccccagccccggcctcccaactcgcaccagccctgtgcggggctccaagagaatggtcagcacctcagctacagacgagcccagggagaccccagggcggccacccgatcccaccggggccccgctgcctggacctacaggggaccctgtgaagcccacatccctggaaaccccctcgccccctctgctgagccgctgcgtctccatgccaggggacatctcaggcttgcagggtggcccccgctccgacttcgacatggcctatgagcgtggccggatctccgtgtccctgcaagaagaggcctccggggggtccctggcagcccccgctcggacccccactcaggagcctcgtgagcagccggcaggcgcctgtgaatacagctactgtgaggatgagtcggccactggtggctgccctttcgggccctaccagggccgccagaccagcagcatcttcgaggcagcaaagcaggagctggccaagctgatgcggattgaggacccctccctcctgaacagcagagtcttgctgcaccacgccaaagctggcaccatcattgcccgccagggagaccaggacgtgagcctgcacttcgtgctctggggctgcctgcacgtgtaccagcgcatgatcgacaaggcggaggacgtgtgcctgttcgtagcgcagcccggggaactggtggggcagctggcggtgctcactggcgaacctctcatcttcacactgcgagcccaacgcgactgcaccttcctgcggatctccaagtccgacttctatgagatcatgcgcgcacagcccagtgtggtgctgagtgcggcgcacacggtggcagccaggatgtcgcccttcgtgcgccagatggacttcgccatcgactggactgcagtggaggcgggacgcgcgctgtacaggcagggcgaccgctccgactgcacttacatcgtgctcaatgggcggctgcgtagcgtgatccagcgaggcagtggcaagaaggagctggtgggcgagtacggccgcggcgacctcatcggcgtggtggaggcactgacccggcagccgcgagccacgacggtgcacgcggtgcgcgacacggagctggccaagcttcccgagggcaccttgggtcacatcaaacgccggtacccgcaggtcgtgacccgccttatccacctactgagccagaaaattctagggaatttgcagcagctgcaaggacccttcccagcaggctctgggttgggtgtgcccccacactcggaactcaccaacccagccagcaacctggcaactgtggcaatcctgcctgtgtgtgctgaggtccccatggtggccttcacgctggagctgcagcacgccctgcaggccatcggtccgacgctactccttaacagtgacatcatccgggcacgcctgggggcctccgcactggatagcatccaagagttccggctgtcagggtggctggcccagcaggaggatgcacaccgtatcgtactctaccagacggacgcctcgctgacgccctggaccgtgcgctgcctgcgacaggccgactgcatcctcattgtgggcctgggggaccaggagcctaccctcggccagctggagcagatgctggagaacacggctgtgcgcgcccttaagcagctagtcctgctccaccgagaggagggcgcgggccccacgcgcaccgtggagtggctaaatatgcgcagctggtgctcggggcacctgcacctgcgctgtccgcgccgcctcttttcgcgccgcagccctgccaagctgcatgagctctacgagaaggttttctccaggcgcgcggaccggcacagcgacttctcccgcttggcgagggtgctcacggggaacaccattgcccttgtgctaggcgggggcggggccaggggctgctcgcacatcggagtactaaaggcattagaggaggcgggggtccccgtggacctggtgggcggcacgtccattggctctttcatcggagcgttgtacgcggaggagcgcagcgccagccgcacgaggcagcgggcccgggagtgggccaagagcatgacttcggtgctggaacctgtgttggacctcacgtacccagtcacctccatgttcactgggtctgcctttaaccgcagcatccatcgggtcttccaggataagcagattgaggacctgtggctgccttacttcaacgtgaccacagatatcaccgcctcagccatgcgagtccacaaagatggctccctgtggcggtacgtgcgcgccagcatgacgctgtcgggctacctgcccccgctgtgcgaccccaaggacgggcacctactcatggatggcggctacatcaacaatctgccagcggacatcgcccgcagcatgggtgccaaaacggtcatcgccattgacgtggggagccaggatgagacggacctcagcacctacggggacagcctgtccggctggtggctgctgtggaagcggctgaatccctgggctgacaaggtaaaggttccagacatggctgaaatccagtcccgcctggcctacgtgtcctgtgtgcggcagctagaggttgtcaagtccagctcctactgcgagtacctgcgcccgcccatcgactgcttcaagaccatggactttgggaagttcgaccagatctatgatgtgggctaccagtacgggaaggcggtgtttggaggctggagccgtggcaacgtcattgagaaaatgctcacagaccggcggtctacagaccttaatgagagccgccgtgcagacgtgcttgccttcccaagctctggcttcactgacttggcagagattgtgtcccggattgagccccccacgagctatgtctctgatggctgtgctgacggagaggagtcagattgtctgacagagtatgaggaggacgccggacccgactgctcgagggatgaaggggggtcccccgagggcgcaagccccagcactgcctccgagatggaggaggagaagtcgattctccggcaacgacgctgtctgccccaggagccgcccggctcagccacagatgcctgaggacctcgacaggggtcaccccctccctcccacccctggactgggctgggggtggccccgtgggggtagctcactccccctcctgctgctatgcctgtgacccccgcggcccacacactggactgacctgccctgagcggggatgcagtgttgcactgatgacttgaccagcccctcccccaataaactcgcctcttggaaaaaaaaaaaaaaaaaaaaaaaaaaaaaaaaaaaaaaaaaa

SEQ ID NO: 58 - protein, 1327 aa, Homo sapiens
MEAPLQTGMVLGVMIGAGVAVVVTAVLILLVVRRLRVPKTPAPDGPRYRFRKRDKVLFYGRKIMRKVSQSTSSLVDTSVSATSRPRMRKKLKMLNIAKKILRIQKETPTLQRKEPPPAVLEADLTEGDLANSHLPSEVLYMLKNVRVLGHFEKPLFLELCRHMVFQRLGQGDYVFRPGQPDASIYVVQDGLLELCLPGPDGKECVVKEVVPGDSVNSLLSILDVITGHQHPQRTVSARAARDSTVLRLPVEAFSAVFTKYPESLVRVVQIIMVRLQRVTFLALHNYLGLTNELFSHEIQPLRLFPSPGLPTRTSPVRGSKRMVSTSATDEPRETPGRPPDPTGAPLPGPTGDPVKPTSLETPSPPLLSRCVSMPGDISGLQGGPRSDFDMAYERGRISVSLQEEASGGSLAAPARTPTQEPREQPAGACEYSYCEDESATGGCPFGPYQGRQTSSIFEAAKQELAKLMRIEDPSLLNSRVLLHHAKAGTIIARQGDQDVSLHFVLWGCLHVYQRMIDKAEDVCLFVAQPGELVGQLAVLTGEPLIFTLRAQRDCTFLRISKSDFYEIMRAQPSVVLSAAHTVAARMSPFVRQMDFAIDWTAVEAGRALYRQGDRSDCTYIVLNGRLRSVIQRGSGKKELVGEYGRGDLIGVVEALTRQPRATTVHAVRDTELAKLPEGTLGHIKRRYPQVVTRLIHLLSQKILGNLQQLQGPFPAGSGLGVPPHSELTNPASNLATVAILPVCAEVPMVAFTLELQHALQAIGPTLLLNSDIIRARLGASALDSIQEFRLSGWLAQQEDAHRIVLYQTDASLTPWTVRCLRQADCILIVGLGDQEPTLGQLEQMLENTAVRALKQLVLLHREEGAGPTRTVEWLNMRSWCSGHLHLRCPRRLFSRRSPAKLHELYEKVFSRRADRHSDFSRLARVLTGNTIALVLGGGGARGCSHIGVLKALEEAGVPVDLVGGTSIGSFIGALYAEERSASRTRQRAREWAKSMTSVLEPVLDLTYPVTSMFTGSAFNRSIHRVFQDKQIEDLWLPYFNVTTDITASAMRVHKDGSLWRYVRASMTLSGYLPPLCDPKDGHLLMDGGYINNLPADIARSMGAKTVIAIDVGSQDETDLSTYGDSLSGWWLLWKRLNPWADKVKVPDMAEIQSRLAYVSCVRQLEVVKSSSYCEYLRPPIDCFKTMDFGKFDQIYDVGYQYGKAVFGGWSRGNVIEKMLTDRRSTDLNESRRADVLAFPSSGFTDLAEIVSRIEPPTSYVSDGCADGEESDCLTEYEEDAGPDCSRDEGGSPEGASPSTASEMEEEKSILRQRRCLPQEPPGSATDA

SEQ ID NO: 59 - DNA, 4445 bp, Homo sapiens
  CDS (1351)..(2883)
gtttcttgctgtgtgaccttgggccaatatctgcactgccctgaccttcagagactagctgccgtcctttcactctctgaggccaggcctgggaaccctcggacaggtgtctgactttgggaaaccctcaagggcttcctgtcacattaatggctctccatccggatctgcaccccttttcctcctccttcgtggctaacttaatgaaaccaagtttgcaaatgaaacataatttcatagacagacatgttgttggaaggtctgggatggtcttaacagctgtctctctaattaccgcagatgctaacgaggtgcctggagcctctggttacaggagcagagctgctgtttgtttgccagggccgggtaggaggcagggctgccaaacctgcccctccattgaggtgtacacacacctgaaggcccttgggcaggcaggacctacagtggaccccatgcccaggctctgggcgggcctcgcctgtgtggccaactcacccagcccagacgtgaacgtttcccagggacagctctccattcactcaattcatccagcaagtgtctgtgatgccccatgcacaggctcagccagtgctagcagtagggtatagtgagcaggccaggcagctcccactccagaggggttgccaggggtgcacaggatccttcagagaacgacagatggcggggagactcagcgaggcagtggtcgggggtacgtgtgctaggcgctccccaggagcctttctgaagagggcacattgggttgggtccacaagggcccatgaagatgccaggggaaatttctggttgtagaggcagcagttgcaaaggccctgaggtgggacaggaggcggttctcatgctacagcgcggggagccggagggtgaggggtcaggtgcccgctgagggcccggggctgtgctgctggccctgtgctgtgcgcttgggtgctggtgaacctccctgggtgggcaagcctcctcaggtgggtatgtcagtatccatgacacaccatagttgtgtcccagagtaatatgggggcccagctgggtggtccctaggaggccagtggatcacagtcacacttggagttgcgtagtatggggtccgcttgtgccatgggcggtgggccatggggagctttgtcctgagcacctccagctggggagcaggcccctgggaggctggagctaggcggggatcctgctgagaccaggggagacttctgggtgaaataggcctcggccctccctgatgcaggtcccgcgtgccacgccatgttcctcgatacactactgcgcctcctggctcatgtgtaatttagggttttcatgtgatattgtgggatggtgggtatgttttgtttcctgattttcttgcagtctctgctgggctttgggactaaggctgtacttgcctcccaaagagttgggaagtgctgctcatttctccttgccaggaacaccatggctggcactcgacgggtggaggggcaggttgggggtaggcccgggggtcctggctgcagcctcatgccgccacccccgcaggagtgcgctggggagccgctgttcatgctgtactgcgccatcaagcagcagatggagaagggccccattgacgccatcacgggtgaggcacgctactccctgagtgaggacaagctcatccggcagcagattgactacaagacactgaccctgaactgtgtgaaccctgagaatgagaatgcacctgaggtgccggtgaaggggctggactgtgacacggtcacccaggccaaggagaagctgctggacgctgcctacaagggcgtgccctactcccagcggcccaaggccgcggacatggacctggagtggcgccagggccgcatggcgcgcatcatcctgcaggacgaggacgtcaccaccaagattgacaacgattggaagaggctgaacacactggctcactaccaggtgacagacgggtcctcggtggcactggtgcccaagcagacgtccgcctacaacatctccaactcctccaccttcaccaagtccctcagcagatacgagagcatgctgcgcacggccagcagccccgacagcctgcgctcgcgcacgcccatgatcacgcccgacctggagagcggcaccaagctgtggcacctggtgaagaaccacgaccacctggaccagcgtgagggtgaccgcggcagcaagatggtctcggagatctacttgacacggctactggccaccaagggcacactgcagaagtttgtggacgacctgtttgagaccatcttcagcacggcacaccggggctcagccctgccgctggccatcaagtacatgttcgacttcctggatgagcaggccgacaagcaccagatccacgatgctgacgtgcgccacacctggaagagcaactgcctgcccctgcgcttctgggtgaacgtgatcaagaacccacagtttgtgttcgacattcacaagaacagcaccacggacgcctgcttgtcggtggtggcccagaccttcatggactcctgctccacctctgagcacaagctgggcaaggactcaccctccaacaagctgctctacgccaaggacatccccaactacaagagctgggtggagaggtactatgcagacatcgccaagatgccagccatcagcgaccaggacatgagtgcgtatctggctgagcagtcccgcctgcacctgagccagttcaacagcatgagcgccttgcacgagatctactcctacatcaccaagtacaaggatgagatcctggcagccctggagaaggatgagcaggcgcggcggcagcggctgcggagcaagctggagcaggtggtggacacgatggccctgagcagctgagccccagctgtgatcatccagcatgatgcagcgtgaggacagctgagcagggaccgggacagccctcaccgcatgcgtgtggagtgtccggtggtgctcgggccgccgcagtgcagcgactgcccggccctccctcccctgcctcacccggtcgggtcccggctcttcctgtgtggaggtgatggtacctgccacaccacagctgcgcacacagctgcttgctcaggggccgggacagcactgggtgctcaggctggccaaggaccttcattgcctggcaagagctgcccagtggccttcatgggagaagggctgacctctgaggggctgaggggtgaggccagggccctccagggggaggggtagccagcttgggctgtccccttgagaccaggacaagaggctgggggtgtcagcattcccagctttccaagctgcccccaggcggcagagtctgagggtcccggggcccggttggcagctggagaaagaggcaaaaagcccgtagccgggcaagaggagctcaagtcggtctgggcccgttgccaccgactcccacctccagcacccatgcccgctgcaccgctgccatcctcagattcaccgcgtgctctgcgcggccgaggccggagcaccacatccacctcgccccagagaggctctgctccctcctatggaggggctgtgggccaggctgctcagactcctgggtggcttccagacggaccgggcagcccctctccgtcctcagggctgtgcctctgggagccactgggccaggggccccgggtcgcagagagcacgttcccgttatttattcccctccgcgtcctacacaggctgccctggcagctgtcttcaagggtaggctgagctccccaccctggagcccctgagggcggcccctgagcactcctctctctccactctctctgtccctgccccagcggcttccagtgtggcatctcagcagtgtcctggcccctccagagcagtgggacatctggggactgtttttgtgtttaggggaaaaaattctgctgcactctgcttgggccttgaggtctgtggcagggctcctctggcccgcagtggcctggatctatctgggccatgagtgacgggcagtgaccagagggactggaggccagcggtgtccacccttgccctcagcaagagagaatgcattcttaaaagaaagctgtacatgtatatatatgcatatatatatatgtggctctagcctcaggctccagccccagtggggtactgtacagttaactgaagaagaattttaaagacgatttgaacaagaaaatgaaggcagtgggaaagcaatgccaaatggttgtggagaaagtggccggagcctccctggagtggagcagccctgaagcctgtgccccccgacctgcgggccgctgttttggtttgacatgacaaggaaaggacttcctgctgaccctgagagcctctggggtgccgcggcaccacggggcatgcatgattgtgctagcgtttagtctgagttgatctttttaaaactgcaagtgttgaatact

SEQ ID NO: 60 - protein, 510 aa, Homo sapiens [sequence omitted]

SEQ ID NO: 61 - DNA, 1440 bp, Homo sapiens
  CDS (1)..(1332)
gattctctgcacaaagcccccaagaagaagagcatcaagtcatccataggccgtctctttggcaagaaagagaagggacgaatgggacccccaggccgggacagctcttctctggctggaacaccctcagatgagacactggccactgaccctctggggctagccaagctgacaggcccaggagacaaggaccgaaggaacaagaggaagcatgaactcctggaggaggcctgccgccagggcctaccttttgctgcctgggacgggcccaccgtggtgtcctggctggagctgtgggtgggcatgcctgcctggtatgtggccgcctgccgggccaatgtcaagagcggtgccatcatggccaacctgtcagacacgaagatccagcgcgagatcggcatcagcaacccgctgcaccgactcaagctacgcctcgccatccaggagatggtctcgctcacctcgccctcagcccccgcctcctcccgcacttccacaggaaacgtgtggatgacacacgaggagatggagtcccttacggccacgaccaagcccgagaccaaggagatcagctgggagcagatcctggcatatggcgacatgaaccacgagtgggtggggaacgactggctgcccagcctggggctgccccaataccgcagctacttcatggagtcgctggtggacgctcgaatgttagatcaccttaacaagaaggagctccggggccaactcaagatggtggacagctttcacagggtgagtctacattatggagttatgtgcctgaaacggctcaactatgaccggaaggacctggagcggaggcgggaagaaagtcagacccagatccgagacgtgatggtgtggtccaatgagcgggtcatgggttgggtgtccgggctgggcctgaaggaatttgccacgaacctcacggagagcggggtacacggggcactgctcgccctggacgagaccttcgactactccgacctggccttgctcctgcagatccccacgcagaatgcacaggcccggcagcttctggagaaggaattcagcaaccttatctccttaggcacagacaggcggctggacgaggacagcgccaagtctttcagccgctccccatcctggcggaagatgttccgggagaaggacctccgaggcgtaactcccgactcagctgagatgttgccccccaactttcgttcggctgcagcgggagccctgggctctccggggctccctctccgcaagctgcagccagaaggccagacttctgggagttcccgggcagacggcgtttcggtccggacctattcctgctagtgcaggcctccaggtgacctcactcggacggaagaatcttcccgaggctgggctgttccctctcctgcccggactgtggcctcgccggggagagcgggcggggagctc

SEQ ID NO: 62 - protein, 443 aa, Homo sapiens
DSLHKAPKKKSIKSSIGRLFGKKEKGRMGPPGRDSSSLAGTPSDETLATDPLGLAKLTGPGDKDRRNKRKHELLEEACRQGLPFAAWDGPTVVSWLELWVGMPAWYVAACRANVKSGAIMANLSDTKIQREIGISNPLHRLKLRLAIQEMVSLTSPSAPASSRTSTGNVWMTHEEMESLTATTKPETKEISWEQILAYGDMNHEWVGNDWLPSLGLPQYRSYFMESLVDARMLDHLNKKELRGQLKMVDSFHRVSLHYGVMCLKRLNYDRKDLERRREESQTQIRDVMVWSNERVMGWVSGLGLKEFATNLTESGVHGALLALDETFDYSDLALLLQIPTQNAQARQLLEKEFSNLISLGTDRRLDEDSAKSFSRSPSWRKMFREKDLRGVTPDSAEMLPPNFRSAAAGALGSPGLPLRKLQPEGQTSGSSRADGVSVRTYSC

SEQ ID NO: 63 - DNA, 2807 bp, Homo sapiens
  CDS (59)..(1792)
caggttgggttctgggacaggtgacccggcggcggggcgaggcagctggcggcgtcgcatggagggctctgggggcggtgcgggcgagcgggcgccgctgctgggcgcgcggcgggcggcggcggccgcggcggcggctggggcgttcgcgggccggcgcgcggcgtgcggggccgtgctgctgacggagctgctggagcgcgccgctttctacggcatcacgtccaacctggtgctattcctgaacggggcgccgttctgctgggagggcgcgcaggccagcgaggcgctgctgctcttcatgggcctcacctacctgggctcgccgttcggaggctggctggccgacgcgcggctgggccgggcgcgcgccatcctgctgagcctggcgctctacctgctgggcatgctggccttcccgctgctggccgcgcccgccacgcgagccgcgctctgcggttccgcgcgcctgctcaactgcacggcgcctggtcccgacgccgccgcccgctgctgctcaccggccaccttcgcggggctggtgctggtgggcctgggcgtggccaccgtcaaggccaacatcacgcccttcggcgccgaccaggttaaagatcgaggtccggaagccactaggagattttttaattggttttattggagcattaacctgggagcgatcctgtcgttaggtggcattgcctatattcagcagaacgtcagctttgtcactggttatgcgatccccactgtctgcgtcggccttgcttttgtggtcttcctctgtggccagagcgttttcatcaccaagcctcctgatggcagtgccttcaccgacatgttcaagatactgacgtattcctgctgttcccagaagcgaagtggagagcgccagagtaatggtgaaggcattggagtctttcagcaatcttctaaacaaagtctgtttgattcatgtaagatgtctcatggtgggccatttacagaagagaaagtggaagatgtgaaagctctggtcaagattgtccctgttttcttggctttgataccttactggacagtgtatttccaaatgcagacaacatatgttttacagagtcttcatttgaggattccagaaatttcaaatattacaaccactcctcacacgctccctgcagcctggctgaccatgtttgatgctgtgctcatcctcctgctcatccctctgaaggacaaactggtcgatcccattttgagaagacatggcctgctcccatcctccctgaagaggatcgccgtgggcatgttctttgtcatgtgctcggcctttgctgcaggaattttggagagtaaaaggctgaaccttgttaaagagaaaaccattaatcagaccatcggcaacgtcgtctaccatgctgccgatctgtcgctgtggtggcaggtgccgcagtacttgctgattgggatcagcgagatctttgcaagtatcgcaggcctggaatttgcatactcagctgcccccaagtccatgcagagtgccataatgggcttgttctttttcttctctggcgtcgggtcgttcgtgggttctggactgctggcactggtgtctatcaaagccatcggatggatgagcagtcacacagactttggtaatattaacggctgctatttgaactattacttttttcttctggctgctattcaaggagctaccctcctgcttttcctcattatttctgtgaaatatgaccatcatcgagaccatcagcgatcaagagccaatggcgtgcccaccagcaggagggcctgaccttcctgaggccatgtgcggtttctgaggctgacatgtcagtaactgactggggtgcactgagaacaggcaagactttaaattcccataaaatgtctgacttcactgaaacttgcatgttgcctggattgatttcttctttccctctatccaaaggagcttggtaagtgccttactgcagcgtgtctcctggcacgctgggccctccgggaggagagctgcagatttcgagtatgtcgcttgtcattcaaggtctctgtgaatcctctagctgggttcccttttttacagaaactcacaaatggagattgcaaagtcttggggaactccacgtgttagttggcatcccagtttcttaaacaaatagtatcacctgcttcccatagccatatctcactgtaaaaaaaaaaaattaataaactgttacttatatttaagaaagtgaggattttttttttttaaagataaaagcatggtcagatgctgcaaggattttacataaatgccatatttatggtttccttcctgagaacagtcttgctcttgccatgttctttgatttaggctggtagtaaacacatttcatctgctgcttcaaaaagtacttactttttaaaccatcaacattacttttctttcttaaggcaaggcatgcataagagtcatttgagaccatgtgtcccatctcaagccacagagcaactcacagggtacttcacaccttacctagtcagagtgcttatatatagctttattttggtacgattgagactaaagactgatcatggttgtatgtaaggaaaacattcttttgaacagaaatagtgtaattaaaaataattgaaagtgttaaatgtgaacttgagctgtttgaccagccacatttttgtattgttactgtacgtgtatctggggcttctccgtttgttaatactttttctgtatttgttgctgtatttttggcataactttattataaaaaaaaaaaaaaaaaaaaaaaaaaaaaaaaaaaaaa

SEQ ID NO: 64 - protein, 577 aa, Homo sapiens
MEGSGGGAGERAPLLGARRAAAAAAAAGAFAGRRAACGAVLLTELLERAAFYGITSNLVLFLNGAPFCWEGAQASEALLLFMGLTYLGSPFGGWLADARLGRARAILLSLALYLLGMLAFPLLAAPATRAALCGSARLLNCTAPGPDAAARCCSPATFAGLVLVGLGVATVKANITPFGADQVKDRGPEATRRFFNWFYWSINLGAILSLGGIAYIQQNVSFVTGYAIPTVCVGLAFVVFLCGQSVFITKPPDGSAFTDMFKILTYSCCSQKRSGERQSNGEGIGVFQQSSKQSLFDSCKMSHGGPFTEEKVEDVKALVKIVPVFLALIPYWTVYFQMQTTYVLQSLHLRIPEISNITTTPHTLPAAWLTMFDAVLILLLIPLKDKLVDPILRRHGLLPSSLKRIAVGMFFVMCSAFAAGILESKRLNLVKEKTINQTIGNVVYHAADLSLWWQVPQYLLIGISEIFASIAGLEFAYSAAPKSMQSAIMGLFFFFSGVGSFVGSGLLALVSIKAIGWMSSHTDFGNINGCYLNYYFFLLAAIQGATLLLFLIISVKYDHHRDHQRSRANGVPTSRRA

SEQ ID NO: 65 - DNA, 1982 bp, Homo sapiens
  CDS (63)..(1460)
ggcgagaggcgggctgaggcggcccagcggcggcaggtgaggcggaaccaaccctcctggccatgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggctctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtctgagtggctgggcggggccggcaggcacagggaggaggtacagaagccggcaacgcttgctatttattttacaaactggactggctcaggcagggccacggctgggctccagctgccggcccagcggatcgtcgcccgatcagtgttttgagggggaaggtggcggggtgggaaccgtgtcattccagagtggatctgcggtgaagccaagccgcaaggttacaaggcatcctcaccaggggccccgcctgctgctcccaggtggcctgcggccactgctatgctcaaggacctggaaacccatgcttcgagacaacgtgactttaatgggagggtgggtgggccgcagacaggctggcagggcaggtgctgcgtggggccctctccagcccgtcctaccctgggctcacatggggcctgtgcccacccctcttgagtgtcttggggacagctctttccacccctggaagatggaaataaacctgcgtgtgggtggagtgttctcgtgccgaattcaaaaagctt

SEQ ID NO: 66 - protein, 465 aa, Homo sapiens
MGGAVVDEGPTGVKAPDGGWGWAVLFGCFVITGFSYAFPKAVSVFFKELIQEFGIGYSDTAWISSILLAMLYGTGPLCSVCVNRFGCRPVMLVGGLFASLGMVAASFCRSIIQVYLTTGVITGLGLALNFQPSLIMLNRYFSKRRPMANGLAAAGSPVFLCALSPLGQLLQDRYGWRGGFLILGGLLLNCCVCAALMRPLVVTAQPGSGPPRPSRRLLDLSVFRDRGFVLYAVAASVMVLGLFVPPVFVVSYAKDLGVPDTKAAFLLTILGFIDIFARPAAGFVAGLGKVRPYSVYLFSFSMFFNGLADLAGSTAGDYGGLVVFCIFFGISYGMVGALQFEVLMAIVGTHKFSSAIGLVLLMEAVAVLVGPPSGGKLLDATHVYMYVFILAGAEVLTSSLILLLGNFFCIRKKPKEPQPEVAAAEEEKLHKPPADSGVDLREVEHFLKAEPEKNGEVVHTPETSV

SEQ ID NO: 67 - DNA, 2856 bp, Homo sapiens
  CDS (591)..(2216)
gtaaccgctactcccggacaccagaccaccgccttccgtacacaggggcccgcatcccaccctcccggacctaagagcctgggtcccctgtttccggaggtccgcttcccggcccccagattctggcatcccagccctcagtgtccaagacccaggcagcccgggtccccgcctcccggatccaggcgtccgggatctgcgccaccagaacctagcctcctgcagacctccgccatctgggggcactcaacctcctggagccaagggccccacgtcccacccagagaaactctcgtattcccagctcctagggccaaggaacccgggcgctccgaactcccagctttcggacatctggcacacggggcagagcagagaagctcagcgcccagcctggggaatttaaacactccagcttccaagagccaaggaacttcagtgctgtgaactcacaactctaaggagccctccaaagttccagtctccaggtgctgttactcaactcagtcctaggaacgtcgggtcctgggaaggagcccaagcgctcccagccagcttccaggcgctaagaaaccccggtgcttcccatcatggtggccgatcctcctcgagactccaaggggctcgcagcggcggagcccaccgccaacgggggcctggcgctggcctccatcgaggaccaaggcgcggcagcaggcggctactgcggttcccgggaccaggtgcgccgctgccttcgagccaacctgcttgtgctgctgacagtggtggccgtggtggccggcgtggcgctgggactgggggtgtcgggggccgggggtgcgctggcgttgggcccggagcgcttgagcgccttcgtcttcccgggcgagctgctgctgcgtctgctgcggatgatcatcttgccgctggtggtgtgcagcttgatcggcggcgccgccagcctggaccccggcgcgctcggccgtctgggcgcctgggcgctgctctttttcctggtcaccacgctgctggcgtcggcgctcggagtgggcttggcgctggctctgcagccgggcgccgcctccgccgccatcaacgcctccgtgggagccgcgggcagtgccgaaaatgcccccagcaaggaggtgctcgattcgttcctggatcttgcgagaaatatcttcccttccaacctggtgtcagcagcctttcgctcatactctaccacctatgaagagaggaatatcaccggaaccagggtgaaggtgcccgtggggcaggaggtggaggggatgaacatcctgggcttggtagtgtttgccatcgtctttggtgtggcgctgcggaagctggggcctgaaggggagctgcttatccgcttcttcaactccttcaatgaggccaccatggttctggtctcctggatcatgtggtacgcccctgtgggcatcatgttcctggtggctggcaagatcgtggagatggaggatgtgggtttactctttgcccgccttggcaagtacattctgtgctgcctgctgggtcacgccatccatgggctcctggtactgcccctcatctacttcctcttcacccgcaaaaacccctaccgcttcctgtggggcatcgtgacgccgctggccactgcctttgggacctcttccagttccgccacgctgccgctgatgatgaagtgcgtggaggagaataatggcgtggccaagcacatcagccgtttcatcctgcccatcggcgccaccgtcaacatggacggtgccgcgctcttccagtgcgtggccgcagtgttcattgcacagctcagccagcagtccttggacttcgtaaagatcatcaccatcctggtcacggccacagcgtccagcgtgggggcagcgggcatccctgctggaggtgtcctcactctggccatcatcctcgaagcagtcaacctcccggtcgaccatatctccttgatcctggctgtggactggctagtcgaccggtcctgtaccgtcctcaatgtagaaggtgacgctctgggggcaggactcctccaaaattatgtggaccgtacggagtcgagaagcacagagcctgagttgatacaagtgaagagtgagctgcccctggatccgctgccagtccccactgaggaaggaaaccccctcctcaaacactatcgggggcccgcaggggatgccacggtcgcctctgagaaggaatcagtcatgtaaaccccgggagggaccttccctgccctgctgggggtgctctttggacactggattatgaggaatggataaatggatgagctagggctctgggggtctgcctgcacactctggggagccaggggccccagcaccctccaggacaggagatctgggatgcctggctgctggagtacatgtgttcacaagggttactcctcaaaacccccagttctcactcatgtccccaactcaaggctagaaaacagcaagatggagaaataatgttctgctgcgtccccaccgtgacctgcctggcctcccctgtctcagggagcaggtcacaggtcaccatggggaattctagcccccactggggggatgttacaacaccatgctggttattttggcggctgtagttgtggggggatgtgtgtgtgcacgtgtgtgtgtgtgtgtgtgtgtgtgtgtgtgtgtgttctgtgacctcctgtccccatggtacgtcccaccctgtccccagatcccctattccctccacaataacagaaacactcccagggactctggggagaggctgaggacaaatacctgctgtcactccagaggacattttttttagcaataaaattgagtgtcaactatttaaaaaaaaaaaaaaaaa

SEQ ID NO: 68 - protein, 541 aa, Homo sapiens
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 69 - DNA, 2445 bp, Homo sapiens
  CDS (467)..(1441)
aggagagtcaggccaatggggccgcagttctttcttttttttttctttattcttatttttggagacagggtctcgctctgttgcccaggctggagtgcggtggtgcgatcacggttccatgcagcccccgacctcccgggctcaggtgattctcccgcctcagcaccgcgagcagctaggaccacaggcgcgagccactgcgtccggccggcgggacttatttgtcaggcggggattgggttccgccagcctaaagggaggggtaagcgccagaatatgaatcgccgggaagctgggagaaagctccgggaaaccctgagcagccaggtcgcctgctccgcccgctcccgctcccgatctctgattgctcctaactgacgtcactcccggtctgtccccgcccactcggtgctgccattggcagtcggtcgtgggtctgagagtcactggagctaccagaagcatcatggggccctggggagagccagagctcctggtgtggcgccccgaggcggtagcttcagagcctccagtgcctgtggggctggaggtgaagttgggggccctggtgctgctgctggtgctcaccctcctctgcagcctggtgcccatctgtgtgctgcgccggccaggagctaaccatgaaggctcagcttcccgccagaaagccctgagcctagtaagctgtttcgcggggggcgtctttttggccacttgtctcctggacctgctgcctgactacctggctgccatagatgaggccctggcagccttgcacgtgacgctccagttcccactgcaagagttcatcctggccatgggcttcttcctggtcctggtgatggagcagatcacactggcttacaaggagcagtcagggccgtcacctctggaggaaacaagggctctgctgggaacagtgaatggtgggccgcagcattggcatgatgggccaggggtcccacaggcgagtggagccccagcaaccccctcagccttgcgtgcctgtgtactggtgttctccctggccctccactccgtgttcgaggggctggcggtagggctgcagcgagaccgggctcgggccatggagctgtgcctggctttgctgctccacaagggcatcctggctgtcagcctgtccctgcggctgttgcagagccaccttagggcacaggtggtggctggctgtgggatcctcttctcatgcatgacacctctaggcatcgggctgggtgcagctctggcagagtcggcaggacctctgcaccagctggcccagtctgtgctagagggcatggcagctggcacctttctctatatcacctttctggaaatcctgccccaggagctggccagttctgagcaaaggatcctcaaggtcattctgctcctagcaggctttgccctgctcactggcctgctcttcatccaaatctagggggcttcaagagaggggcaggggagattgatgatcaggtgcccctgttctcccttccctcccccagttgtggggaataggaaggaaaggggaagggaaatactgaggaccaaaaagttctctgggagctaaagatagagcctttggggctatctgactaatgagagggaagtgggcagacaagaggctggccccagtcccaaggaacaagagatggtcaagtcgctagagacatatcaggggacattaggattggggaagacacttgactgctagaatcagaggttggacactatacataaggacaggctcacatgggaggctggaggtgggtacccagctgctgtggaacgggtatggacaggtcataaacctagagtcagtgtcctgttggtcctagcccatttcagcaccctgccacttggagtggacccctcctactcttcttagcgcctaccctcatacctatctccctcctcccatctcctaggggactggcgccaaatggtctctccctgccaattttggtatcttctctggcctctccagtcctgcttactcctctatttttaaagtgccaaacaaatccccttcctctttctcaaagcacagtaatgtggcactgagccctacccagcacctcagtgaagggggcctgcttgctctttattttggtcccggatcctggggtggggcagaaatattttctgggctggggtaggaggaaggttgttgcagccatctactgctgctgtaccctaggaatatggggacatggacatggtgtcccatgcccagatgataaacactgagctgccaaaacatttttttaaatacacccgaggagcccaagggggaagggcaatgcctacccccagcgttatttttggggagggagggctgtgcatagggacatattctttagaatctattttattaactgacctgttttgggacctgttacccaaataaaagatgtttctagaaaaaaaaaaaaaaaaaaa

SEQ ID NO: 70 - protein, 324 aa, Homo sapiens
MGPWGEPELLVWRPEAVASEPPVPVGLEVKLGALVLLLVLTLLCSLVPICVLRRPGANHEGSASRQKALSLVSCFAGGVFLATCLLDLLPDYLAAIDEALAALHVTLQFPLQEFILAMGFFLVLVMEQITLAYKEQSGPSPLEETRALLGTVNGGPQHWHDGPGVPQASGAPATPSALRACVLVFSLALHSVFEGLAVGLQRDRARAMELCLALLLHKGILAVSLSLRLLQSHLRAQVVAGCGILFSCMTPLGIGLGAALAESAGPLHQLAQSVLEGMAAGTFLYITFLEILPQELASSEQRILKVILLLAGFALLTGLLFIQI

SEQ ID NO: 71 - DNA, 1544 bp, Homo sapiens
  CDS (301)..(1059)
gcacgagttgggaggtgtagcgcggctctgaacgcgctgagggccgttgagtgtcgcaggcggcgagggcgcgagtgaggagcagacccaggcatcgcgcgccgagaaggccgggcgtccccacactgaaggtccggaaaggcgacttccgggggctttggcacctggcggaccctcccggagcgtcggcacctgaacgcgaggcgctccattgcgcgtgcgcgttgaggggcttcccgcacctgatcgcgagaccccaacggctggtggcgtcgcctgcgcgtctcggctgagctggccatggcgcagctgtgcgggctgaggcggagccgggcgtttctcgccctgctgggatcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggcggggctgttcgtgatggtgttgatcctcttcctgggagcctccatggtctacctgatccgggtggcacggaggaaccaggagcgtgccctgcgcaccgtctggagctccggagatgacaaggagcagctggtgaagaacacatatgtcctgtgaccgccctgtcgccaagaggactggggaagggaggggagactatgtgtgagctttttttaaatagagggattgactcggatttgagtgatcattagggctgaggtctgtttctctgggaggtaggacggctgcttcctggtctggcagggatgggtttgctttggaaatcctctaggaggctcctcctcgcatggcctgcagtctggcagcagccccgagttgtttcctcgctgatcgatttctttcctccaggtagagttttctttgcttatgttgaattccattgcctccttttctcnatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaatttccagcatgttgctttcaaaaaaaaaaaaaaaaaaa

SEQ ID NO: 72 - protein, 252 aa, Homo sapiens
MAQLCGLRRSRAFLALLGSLLLSGVLAADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSSGDDKEQLVKNTYVL

SEQ ID NO: 73 - DNA, 2380 bp, Homo sapiens
  CDS (135)..(1043)
gaggaggagggaaaaggcgagcaaaaaggaagagtgggaggaggaggggaagcggcgaaggaggaagaggaggaggaggaagaggggagcacaaaggatccaggtctcccgacgggaggttaataccaagaaccatgtgtgccgagcggctgggccagttcatgaccctggctttggtgttggccacctttgacccggcgcgggggaccgacgccaccaacccacccgagggtccccaagacaggagctcccagcagaaaggccgcctgtccctgcagaatacagcggagatccagcactgtttggtcaacgctggcgatgtggggtgtggcgtgtttgaatgtttcgagaacaactcttgtgagattcggggcttacatgggatttgcatgacttttctgcacaacgctggaaaatttgatgcccagggcaagtcattcatcaaagacgccttgaaatgtaaggcccacgctctgcggcacaggttcggctgcataagccggaagtgcccggccatcagggaaatggtgtcccagttgcagcgggaatgctacctcaagcacgacctgtgcgcggctgcccaggagaacacccgggtgatagtggagatgatccatttcaaggacttgctgctgcacgaaccctacgtggacctcgtgaacttgctgctgacctgtggggaggaggtgaaggaggccatcacccacagcgtgcaggttcagtgtgagcagaactggggaagcctgtgctccatcttgagcttctgcacctcggccatccagaagcctcccacggcgccccccgagcgccagccccaggtggacagaaccaagctctccagggcccaccacggggaagcaggacatcacctcccagagcccagcagtagggagactggccgaggtgccaagggtgagcgaggtagcaagagccacccaaacgcccatgcccgaggcagagtcgggggccttggggctcagggaccttccggaagcagcgagtgggaagacgaacagtctgagtattctgatatccggaggtgaaatgaaaggcctggccacgaaatctttcctccacgccgtccattttcttatctatggacattccaaaacatttaccattagagaggggggatgtcacacgcaggattctgtggggactgtggacttcatcgaggtgtgtgttcgcggaacggacaggtgagatggagacccctggggccgtggggtctcaggggtgcctggtgaattctgcacttacacgtactcaagggagcgcgcccgcgttatcctcgtacctttgtcttctttccatctgtggagtcagtgggtgtcggccgctctgttgtgggggaggtgaaccagggaggggcagggcaaggcagggcccccagagctgggccacacagtgggtgctgggcctcgccccgaagcttctggtgcagcagcctctggtgctgtctccgcggaagtcagggcggctggattccaggacaggagtgaatgtaaaaataaatatcgcttagaatgcaggagaagggtggagaggaggcaggggccgagggggtgcttggtgccaaactgaaattcagtttcttgtgtggggccttgcggttcagagctcttggcgagggtggagggaggagtgtcatttctatgtgtaatttctgagccattgtactgtctgggctgggggggacactgtccaagggagtggcccctatgagtttatattttaaccactgcttcaaatctcgatttcactttttttatttatccagttatatctacatatctgtcatctaaataaatggctttcaaacaaagcaactgggtcattaaaaccagctcaaagggggtttaaaaaaaaaaaaaccagcccatcctttgaggctgatttttcttttttttaagttctattttaaaagctatcaaacagcgacatagccatacatctgactgcctgacatggactcctgcccacttgggggaaaccttatacccagaggaaaatacacacctggggagtacatttgacaaatttcccttaggatttcgttatctcaccttgaccctcagccaagattggtaaagctgcgtcctggcgattccaggagacccagctggaaacctggcttctccatgtgaggggatgggaaaggaaagaagagaatgaagactacttagtaattcccatcaggaaatgctgaccttttacataaaatcaaggagactgctgaaaatctctaagggacaggattttccagatcctaattggaaatttagcaataaggagaggagtccaaggggacaaataaaggcagagagagagagagagagagggagaggaagaaaagagagagagaaaagagcctcgtgcc

SEQ ID NO: 74 - protein, 302 aa, Homo sapiens
MCAERLGQFMTLALVLATFDPARGTDATNPPEGPQDRSSQQKGRLSLQNTAEIQHCLVNAGDVGCGVFECFENNSCEIRGLHGICMTFLHNAGKFDAQGKSFIKDALKCKAHALRHRFGCISRKCPAIREMVSQLQRECYLKHDLCAAAQENTRVIVEMIHFKDLLLHEPYVDLVNLLLTCGEEVKEAITHSVQVQCEQNWGSLCSILSFCTSAIQKPPTAPPERQPQVDRTKLSRAHHGEAGHHLPEPSSRETGRGAKGERGSKSHPNAHARGRVGGLGAQGPSGSSEWEDEQSEYSDIRR

SEQ ID NO: 75 - DNA, 3662 bp, Homo sapiens
  CDS (434)..(2401)
gccaccacgtgtgtccctgcgcccggtggccaccgactcagtccctcgccgaccagtctgggcagcggaggagggtggttggcagtggctggaagcttcgctatgggaagttgttcctttgctctctcgcgcccagtcctcctccctggttctcctcagccgctgtcggaggagagcacccggagacgcgggctgcagtcgcggcggcttctccccgcctgggcggccgcgccgctgggcaggtgctgagcgcccctagagcctcccttgccgcctccctcctctgcccggccgcagcagtgcacatggggtgttggaggtagatgggctcccggcccgggaggcggcggtggatgcggcgctgggcagaagcagccgccgattccagctgccccgcgcgccccgggcgcccctgcgagtccccggttcagccatggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatccggaaaagctcgaggactctgaaaaaggggccccggcaggatcccagtgccattgtggaaaaggcagggctgaagaaatccatgactccaacccagaaccgggagaaatggatctactactgcaatggccatggtatcgatatcctgaagcttgtagcagcccaagtgggaagccagtggaaagatatctatcagtttctttgcaatgccagtgagagggaggttgctgctttctccaatgggtacacagccgaccacgagcgggcctacgcagctctgcagcactggaccatccggggccccgaggccagcctcgcccagctaattagcgccctgcgccagcaccggagaaacgatgttgtggagaagattcgtgggctgatggaagacaccacccagctggaaactgacaaactagctctcccgatgagccccagcccgcttagcccgagccccatccccagccccaacgcgaaacttgagaattccgctctcctgacggtggagccttccccacaggacaagaacaagggcttcttcgtggatgagtcggagccccttctccgctgtgactctacatccagcggctcctccgcgctgagcaggaacggttcctttattaccaaagaaaagaaggacacagtgttgcggcaggtacgcctggacccctgtgacttgcagcctatctttgatgacatgctccactttctaaatcctgaggagctgcgggtgattgaagagattccccaggctgaggacaaactagaccggctattcgaaattattggagtcaagagccaggaagccagccagaccctcctggactctgtttatagccatcttcctgacctgctgtagaacatagggatactgcattctggaaattactcaatttagtggcagggtggttttttaattttcttctgtttctgatttttgttgtttggggtgtgtgtgtgtgtttgtgtgtgtgtgtgtgtgtgtgtgtgtgtgtgtgtttaacagagaatatggccagtgcttgagttctttctccttctctctctctcttttttttttaaataactcttctgggaagttggtttataagcctttgccaggtgtaactgttgtgaaatacccaccactaaagttttttaagttccatattttctccattttgccttcttatgtattttcaagattattctgtgcactttaaatttacttaacttaccataaatgcagtgtgacttttcccacacactggattgtgaggctcttaacttcttaaaagtataatggcatcttgtgaatcctataagcagtctttatgtctcttaacattcacacctactttttaaaaacaaatattattactatttttattattgtttgtcctttataaattttcttaaagattaagaaaatttaagaccccattgagttactgtaatgcaattcaactttgagttatcttttaaatatgtcttgtatagttcatattcatggctgaaacttgaccacactattgctgattgtatggttttcacctggacaccgtgtagaatgcttgattacttgtactcttcttatgctaatatgctctgggctggagaaatgaaatcctcaagccatcaggatttgctatttaagtggcttgacaactgggccaccaaagaacttgaacttcaccttttaggatttgagctgttctggaacacattgctgcactttggaaagtcaaaatcaagtgccagtggcgccctttccatagagaatttgcccagctttgctttaaaagatgtcttgttttttatatacacataatcaataggtccaatctgctctcaaggccttggtcctggtgggattccttcaccaattactttaattaaaaatggctgcaactgtaagaacccttgtctgatatatttgcaactatgctcccatttacaaatgtaccttctaatgctcagttgccaggttccaatgcaaaggtggcgtggactccctttgtgtgggtggggtttgtgggtagtggtgaaggaccgatatcagaaaaatgccttcaagtgtactaatttattaataaacattaggtgtttgttaaaaaaaaaaaaaaaaaaaaaaaaaaaaa

SEQ ID NO: 76 - protein, 655 aa, Homo sapiens
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 77 - DNA, 2780 bp, Homo sapiens
  CDS (106)..(2517)
gtgggcggaccgcgcggctggaggtgtgaggatccgaacccaggggtggggggtggaggcggctcctgcgatcgaaggggacttgagactcaccggccgcacgccatgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaaattatactctcaccatttggatcctgtgtggagagggaatgtgaaatttacatcatttctttttgggagagacttgttttggatgccccctaatccccttctcccctgcactgtaaaatgtgggattatgggtcacaggaaaaagtgggttttttagttgaattttttttaacattcctcatgaatgtaaatttgtactatttaactgactattcttgatgtaaaatcttgtcatgtgtataaaaataaaaaagatcccaaat

SEQ ID NO: 78 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 79 - DNA, 4061 bp, Homo sapiens
  CDS (73)..(3717)
ggtctggaagcagagccggcggagggagcgccggggccctgggctgcaggaggttgcggcggccgcggcagcatggtggtgccggagaaggagcagagctggatccccaagatcttcaagaagaagacctgcacgacgttcatagttgactccacagatccgggagggaccttgtgccagtgtgggcgcccccggaccgcccaccccgcagtggccatggaggatgccttcggggcagccgtggtgaccgtgtgggacagcgatgcacacaccacggagaagcccaccgatgcctacggagagctggacttcacgggggccggccgcaagcacagcaatttcctccggctctctgaccgaacggatccagctgcagtttatagtctggtcacacgcacatggggcttccgtgccccgaacctggtggtgtcagtgctggggggatcggggggccccgtcctccagacctggctgcaggacctgctgcgtcgtgggctggtgcgggctgcccagagcacaggagcctggattgtcactgggggtctgcacacgggcatcggccggcatgttggtgtggctgtacgggaccatcagatggccagcactgggggcaccaaggtggtggccatgggtgtggccccctggggtgtggtccggaatagagacaccctcatcaaccccaagggctcgttccctgcgaggtaccggtggcgcggtgacccggaggacggggtccagtttcccctggactacaactactcggccttcttcctggtggacgacggcacacacggctgcctggggggcgagaaccgcttccgcttgcgcctggagtcctacatctcacagcagaagacgggcgtgggagggactggaattgacatccctgtcctgctcctcctgattgatggtgatgagaagatgttgacgcgaatagagaacgccacccaggctcagctcccatgtctcctcgtggctggctcagggggagctgcggactgcctggcggagaccctggaagacactctggccccagggagtgggggagccaggcaaggcgaagcccgagatcgaatcaggcgtttctttcccaaaggggaccttgaggtcctgcaggcccaggtggagaggattatgacccggaaggagctcctgacagtctattcttctgaggatgggtctgaggaattcgagaccatagttttgaaggcccttgtgaaggcctgtgggagctcggaggcctcagcctacctggatgagctgcgtttggctgtggcttggaaccgcgtggacattgcccagagtgaactctttcggggggacatccaatggcggtccttccatctcgaagcttccctcatggacgccctgctgaatgaccggcctgagttcgtgcgcttgctcatttcccacggcctcagcctgggccacttcctgaccccgatgcgcctggcccaactctacagcgcggcgccctccaactcgctcatccgcaaccttttggaccaggcgtcccacagcgcaggcaccaaagccccagccctaaaagggggagctgcggagctccggccccctgacgtggggcatgtgctgaggatgctgctggggaagatgtgcgcgccgaggtacccctccgggggcgcctgggaccctcacccaggccagggcttcggggagagcatgtatctgctctcggacaaggccacctcgccgctctcgctggatgctggcctcgggcaggccccctggagcgacctgcttctttgggcactgttgctgaacagggcacagatggccatgtacttctgggagatgggttccaatgcagtttcctcagctcttggggcctgtttgctgctccgggtgatggcacgcctggagcctgacgctgaggaggcagcacggaggaaagacctggcgttcaagtttgaggggatgggcgttgacctctttggcgagtgctatcgcagcagtgaggtgagggctgcccgcctcctcctccgtcgctgcccgctctggggggatgccacttgcctccagctggccatgcaagctgacgcccgtgccttctttgcccaggatggggtacagtctctgctgacacagaagtggtggggagatatggccagcactacacccatctgggccctggttctcgccttcttttgccctccactcatctacacccgcctcatcaccttcaggaaatcagaagaggagcccacacgggaggagctagagtttgacatggatagtgtcattaatggggaagggcctgtcgggacggcggacccagccgagaagacgccgctgggggtcccgcgccagtcgggccgtccgggttgctgcgggggccgctgcggggggcgccggtgcctacgccgctggttccacttctggggcgcgccggtgaccatcttcatgggcaacgtggtcagctacctgctgttcctgctgcttttctcgcgggtgctgctcgtggatttccagccggcgccgcccggctccctggagctgctgctctatttctgggctttcacgctgctgtgcgaggaactgcgccagggcctgagcggaggcgggggcagcctcgccagcgggggccccgggcctggccatgcctcactgagccagcgcctgcgcctctacctcgccgacagctggaaccagtgcgacctagtggctctcacctgcttcctcctgggcgtgggctgccggctgaccccgggtttgtaccacctgggccgcactgtcctctgcatcgacttcatggttttcacggtgcggctgcttcacatcttcacggtcaacaaacagctggggcccaagatcgtcatcgtgagcaagatgatgaaggacgtgttcttcttcctcttcttcctcggcgtgtggctggtagcctatggcgtggccacggaggggctcctgaggccacgggacagtgacttcccaagtatcctgcgccgcgtcttctaccgtccctacctgcagatcttcgggcagattccccaggaggacatggacgtggccctcatggagcacagcaactgctcgtcggagcccggcttctgggcacaccctcctggggcccaggcgggcacctgcgtctcccagtatgccaactggctggtggtgctgctcctcgtcatcttcctgctcgtggccaacatcctgctggtcaacttgctcattgccatgttcagttacacattcggcaaagtacagggcaacagcgatctctactggaaggcgcagcgttaccgcctcatccgggaattccactctcggcccgcgctggccccgccctttatcgtcatctcccacttgcgcctcctgctcaggcaattgtgcaggcgaccccggagcccccagccgtcctccccggccctcgagcatttccgggtttacctttctaaggaagccgagcggaagctgctaacgtgggaatcggtgcataaggagaactttctgctggcacgcgctagggacaagcgggagagcgactccgagcgtctgaagcgcacgtcccagaaggtggacttggcactgaaacagctgggacacatccgcgagtacgaacagcgcctgaaagtgctggagcgggaggtccagcagtgtagccgcgtcctggggtgggtggccgaggccctgagccgctctgccttgctgcccccaggtgggccgccaccccctgacctgcctgggtccaaagactgagccctgctggcggacttcaaggagaagcccccacaggggattttgctcctagagtaaggctcatctgggcctcggcccccgcacctggtggccttgtccttgaggtgagccccatgtccatctgggccactgtcaggaccacctttgggagtgtcatccttacaaaccacagcatgcccggctcctcccagaaccagtcccagcctgggaggatcaaggcctggatcccgggccgttatccatctggaggctgcagggtccttggggtaacagggaccacagacccctcaccactcacagattcctcacactggggaaataaagccatttcagaggaaaaaaaa

SEQ ID NO: 80 - protein, 1214 aa, Homo sapiens
MVVPEKEQSWIPKIFKKKTCTTFIVDSTDPGGTLCQCGRPRTAHPAVAMEDAFGAAVVTVWDSDAHTTEKPTDAYGELDFTGAGRKHSNFLRLSDRTDPAAVYSLVTRTWGFRAPNLVVSVLGGSGGPVLQTWLQDLLRRGLVRAAQSTGAWIVTGGLHTGIGRHVGVAVRDHQMASTGGTKVVAMGVAPWGVVRNRDTLINPKGSFPARYRWRGDPEDGVQFPLDYNYSAFFLVDDGTHGCLGGENRFRLRLESYISQQKTGVGGTGIDIPVLLLLIDGDEKMLTRIENATQAQLPCLLVAGSGGAADCLAETLEDTLAPGSGGARQGEARDRIRRFFPKGDLEVLQAQVERIMTRKELLTVYSSEDGSEEFETIVLKALVKACGSSEASAYLDELRLAVAWNRVDIAQSELFRGDIQWRSFHLEASLMDALLNDRPEFVRLLISHGLSLGHFLTPMRLAQLYSAAPSNSLIRNLLDQASHSAGTKAPALKGGAAELRPPDVGHVLRMLLGKMCAPRYPSGGAWDPHPGQGFGESMYLLSDKATSPLSLDAGLGQAPWSDLLLWALLLNRAQMAMYFWEMGSNAVSSALGACLLLRVMARLEPDAEEAARRKDLAFKFEGMGVDLFGECYRSSEVRAARLLLRRCPLWGDATCLQLAMQADARAFFAQDGVQSLLTQKWWGDMASTTPIWALVLAFFCPPLIYTRLITFRKSEEEPTREELEFDMDSVINGEGPVGTADPAEKTPLGVPRQSGRPGCCGGRCGGRRCLRRWFHFWGAPVTIFMGNVVSYLLFLLLFSRVLLVDFQPAPPGSLELLLYFWAFTLLCEELRQGLSGGGGSLASGGPGPGHASLSQRLRLYLADSWNQCDLVALTCFLLGVGCRLTPGLYHLGRTVLCIDFMVFTVRLLHIFTVNKQLGPKIVIVSKMMKDVFFFLFFLGVWLVAYGVATEGLLRPRDSDFPSILRRVFYRPYLQIFGQIPQEDMDVALMEHSNCSSEPGFWAHPPGAQAGTCVSQYANWLVVLLLVIFLLVANILLVNLLIAMFSYTFGKVQGNSDLYWKAQRYRLIREFHSRPALAPPFIVISHLRLLLRQLCRRPRSPQPSSPALEHFRVYLSKEAERKLLTWESVHKENFLLARARDKRESDSERLKRTSQKVDLALKQLGHIREYEQRLKVLEREVQQCSRVLGWVAEALSRSALLPPGGPPPPDLPGSKD

SEQ ID NO: 81 - DNA, 27 bp, Artificial Sequence
  primer
accatggcctcaccgttgacccgcttt

SEQ ID NO: 82 - DNA, 25 bp, Artificial Sequence
  primer
ctagcggctgtggtagcagatgaga

SEQ ID NO: 83 - DNA, 29 bp, Artificial Sequence
  primer
ctacggatccaccatggcctcaccgttga

SEQ ID NO: 84 - DNA, 35 bp, Artificial Sequence
  primer
gtacatcgatctagcggctgtggtagcagatgaga

SEQ ID NO: 85 - DNA, 61 bp, Artificial Sequence
  primer
agctgtaaaacgacggccagtgagcgtttaaacgaattccagactagtggccggccgtgca

SEQ ID NO: 86 - DNA, 53 bp, Artificial Sequence
  primer
cggccggccactagtctggaattcgtttaaacgctcactggccgtcgttttac

SEQ ID NO: 87 - DNA, 32 bp, Artificial Sequence
  primer
aattctgcagcccaggtaaaattcgctagcct

SEQ ID NO: 88 - DNA, 32 bp, Artificial Sequence
  primer
ctagaggctagcgaattttacctgggctgcag

SEQ ID NO: 89 - DNA, 72 bp, Artificial Sequence
  primer
cggtccgtgagtgagtgaggcgcgccggatcctaacctaggtaatcatggtcatagctgtttcctgcagggc

SEQ ID NO: 90 - DNA, 80 bp, Artificial Sequence
  primer
ggccgccctgcaggaaacagctatgaccatgattacctaggttaggatccggcgcgcctcactcactcacggaccgtgca

SEQ ID NO: 91 - DNA, 35 bp, Artificial Sequence
  primer
gatcccgggtcgtgtattcagctttccttgttcct

SEQ ID NO: 92 - DNA, 35 bp, Artificial Sequence
  primer
ctagaggaacaaggaaagctgaatacacgacccgg

SEQ ID NO: 93 - DNA, 54 bp, Artificial Sequence
  primer
catcaagcttggccggccaccatggacgcgtccgaagacgccaaaaacataaag

SEQ ID NO: 94 - DNA, 35 bp, Artificial Sequence
  primer
cacgtggatatcttacaatttggactttccgccct

SEQ ID NO: 95 - DNA, 31 bp, Artificial Sequence
  primer
ttgtaagatatccacgtgttgacaattaatc

SEQ ID NO: 96 - DNA, 45 bp, Artificial Sequence
  primer
catcagatctgtcgaccggaccgacgcgtccacgaagtgcttagc

SEQ ID NO: 97 - DNA, 54 bp, Artificial Sequence; identical to SEQ ID NO: 93
  primer

SEQ ID NO: 98 - DNA, 45 bp, Artificial Sequence; identical to SEQ ID NO: 96
  primer

SEQ ID NO: 99 - DNA, 43 bp, Artificial Sequence
  primer
catcaagcttggccggccacgcgtgttggtaaaatggaagacg

SEQ ID NO: 100 - DNA, 40 bp, Artificial Sequence
  primer
catgagatctgtcgaccggaccgccacgaagtgcttaagc

SEQ ID NO: 101 - DNA, 35 bp, Artificial Sequence
  primer
gtaatacgactcactataggcgcgccnnnnnnnnn

SEQ ID NO: 102 - DNA, 36 bp, Artificial Sequence
  primer
gtaatacgactcactataggcggaccgnnnnnnnnn

SEQ ID NO: 103 - DNA, 21 bp, Artificial Sequence
  primer
atgattacgccacggaccgtc

SEQ ID NO: 104 - DNA, 30 bp, Artificial Sequence
  primer
gacggtccgtggcgtaatcatggtcatagc

SEQ ID NO: 105 - DNA, 22 bp, Artificial Sequence
  primer
atgattacgccaggcgcgccac

SEQ ID NO: 106 - DNA, 31 bp, Artificial Sequence
  primer
gtggcgcgcctggcgtaatcatggtcatagc

SEQ ID NO: 107 - DNA, 30 bp, Artificial Sequence
  primer
gctatgaccatgattacgccacggaccgtc

SEQ ID NO: 108 - DNA, 21 bp, Artificial Sequence
  primer
gtaatacgactcactataggc

SEQ ID NO: 109 - DNA, 31 bp, Artificial Sequence
  primer
gctatgaccatgattacgccaggcgcgccac

SEQ ID NO: 110 - DNA, 25 bp, Artificial Sequence
  primer
gtaatacgactcactataggcggac

SEQ ID NO: 111 - DNA, 30 bp, Artificial Sequence; identical to SEQ ID NO: 107
  primer

SEQ ID NO: 112 - DNA, 21 bp, Artificial Sequence
  primer
gtaatacgactcactataggc

SEQ ID NO: 113 - DNA, 31 bp, Artificial Sequence; identical to SEQ ID NO: 109
  primer

SEQ ID NO: 114 - DNA, 25 bp, Artificial Sequence
  primer
gtaatacgactcactataggcggac

SEQ ID NO: 115 - DNA, 34 bp, Artificial Sequence
  primer
tctgcagcccaggtaaaattcgctagcctctagt

SEQ ID NO: 116 - DNA, 35 bp, Artificial Sequence
  primer
gaggaacaaggaaagctgaatacacgacccgtgat

SEQ ID NO: 117 - DNA, 17 bp, Artificial Sequence
  primer
gtaaaacgacggccagt

SEQ ID NO: 118 - DNA, 34 bp, Artificial Sequence; identical to SEQ ID NO: 115
  primer

SEQ ID NO: 119 - DNA, 25 bp, Artificial Sequence
  primer
tcgttcgaggagcccttggcagcat

SEQ ID NO: 120 - DNA, 24 bp, Artificial Sequence
  primer
cgcccttccgccacggccgtctct

SEQ ID NO: 121 - DNA, 25 bp, Artificial Sequence
  primer
gaaaggacccgtcgccatgggccgt

SEQ ID NO: 122 - DNA, 25 bp, Artificial Sequence
  primer
cagtcgccaatatgcagctctttgt

SEQ ID NO: 123 - DNA, 20 bp, Artificial Sequence
  primer
cgaggtatgctgccccacaa

SEQ ID NO: 124 - DNA, 25 bp, Artificial Sequence
  primer
cgagcgcctgtgcacagcagccaga

SEQ ID NO: 125 - DNA, 25 bp, Artificial Sequence
  primer
gcgggacatgattcgggaggtgtgt

SEQ ID NO: 126 - DNA, 25 bp, Artificial Sequence
  primer
ctgcgcgcctgcgcgccgtggattt

SEQ ID NO: 127 - DNA, 25 bp, Artificial Sequence
  primer
cttcgaggtgaccggccaggaaacg

SEQ ID NO: 128 - DNA, 25 bp, Artificial Sequence
  primer
caggccgctctggaccgtctcaagg

SEQ ID NO: 129 - DNA, 25 bp, Artificial Sequence
  primer
aacggtgggcttgttgctgctctgg

SEQ ID NO: 130 - DNA, 25 bp, Artificial Sequence
  primer
attggtattggtaacgggcgtcagg

SEQ ID NO: 131 - DNA, 25 bp, Artificial Sequence
  primer
accatcttccaggcgcagttgagtt